SEQUENCE LISTING (2 sequences)

SEQ ID NO: 1 - protein, 645 aa, Homo Sapiens
HGEGTFTSDVSSYLEGQAAKEFIAWLVKGRHGEGTFTSDVSSYLEGQAAKEFIAWLVKGRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 2 - protein, 31 aa, Homo Sapiens
  VARIANT 31: Xaa = Any Amino Acid
HAEGTFTSDVSSYLEGQAAKEFIAWLVKGRX

(19) United States
(12) Patent Application Publication
(10) Pub. No.: US 2019/0175701 A1
(43) Pub. Date: Jun. 13, 2019

(54) Pharmaceutical Compositions

(71) Applicant: GlaxoSmithKline LLC, Wilmington, DE (US)

(72) Inventors: Liuquan CHANG, Old Tappan, NJ (US); Dany Doucet, King of Prussia, PA (US); Douglas P. Nesta, King of Prussia, PA (US)

(21) Appl. No.: 16/284,017

(22) Filed: Feb. 25, 2019

Related U.S. Application Data
(63) Continuation of application No. 16/127,393, filed on Sep. 11, 2018, now abandoned.
Application No. 16/127,393 is a continuation of application No. 15/318,095, filed on Dec. 12, 2016, now abandoned.
(60) Provisional application No. 62/016,806, filed on Jun. 25, 2014.

(51) Int. Cl. A61K 38/26; C07K 19/00; A61K 47/22; C07K 14/605; C07K 14/76; A61K 9/00; A61K 9/08; A61K 47/26; A61K 47/18; A61K 47/12

(52) U.S. Cl. CPC A61K 38/26; C07K 19/00; A61K 47/22; C07K 14/605; C07K 14/76; C07K 2319/00; A61K 9/08; A61K 47/26; A61K 47/183; A61K 47/12; A61K 9/0019

(57) ABSTRACT

The present invention provides liquid compositions comprising albiglutide or a variant thereof, a buffering agent, at least one saccharide and/or at least one polyol, at least one stabilizing agent and optionally a surfactant wherein said albiglutide remains stable in said liquid composition. Albiglutide or a variant thereof can be considered to remain stable in liquid if at least about ≥96% of said albiglutide or a variant thereof remains as a monomer in the liquid composition over a period of at least one week.

This application is a continuation of U.S. application Ser. No. 15/318,095 filed on Dec. 12, 2016, which is a 371 of International PCT/US2015/037183 filed on Jun. 23, 2015, which claims the benefit of U.S. Provisional Application 62/016,806 filed on Jun. 25, 2014, all of which are incorporated by reference herein in their entireties.

### FIELD OF THE INVENTION

The present invention relates to liquid compositions comprising GLP-1 agonists, including albiglutide.

### BACKGROUND

Hypoglycemic agents may be used in the treatment of both type 1 and type 2 diabetes to lower glucose concentration in blood. Insulinotropic peptides such as exendin-4 and GLP-1 derivatives are currently sold as therapeutic agents for the treatment of diabetes. Products include BYETTA® and BYDUREON® (exendin-4 or Exenatide); VICTOZA® (liraglutide; GLP-1 fragment fused with palmitoyl); TRULICITY™ (dulaglutide; GLP-1 analog fused to an IgG4 Fc region) and TANZEUM™/EPERZAN™. Insulinotropic peptides include, but are not limited to, incretin hormones, for example, gastric inhibitory peptide (GIP) and glucagon like peptide-1 (GLP-1), as well as fragments, variants, and/or conjugates thereof. Insulinotropic peptides also include, for example, exendin-3 and exendin-4. GLP-1 is a 36 amino acid long incretin hormone secreted by the L-cells in the intestine in response to ingestion of food. GLP-1 has been shown to stimulate insulin secretion in a physiological and glucose-dependent manner, decrease glucagon secretion, inhibit gastric emptying, decrease appetite, and stimulate proliferation of β-cells. In non-clinical experiments GLP-1 promotes continued beta cell competence by stimulating transcription of genes important for glucose dependent insulin secretion and by promoting beta-cell neogenesis (Meier, et al. Biodrugs. 2003; 17 (2): 93-102).

In a healthy individual, GLP-1 plays an important role regulating post-prandial blood glucose levels by stimulating glucose-dependent insulin secretion by the pancreas resulting in increased glucose absorption in the periphery. GLP-1 also suppresses glucagon secretion, leading to reduced hepatic glucose output. In addition, GLP-1 delays gastric emptying and slows small bowel motility delaying food absorption. In people with type 2 diabetes mellitus (T2DM), the normal post-prandial rise in GLP-1 is absent or reduced (Vilsboll T, et al., Diabetes. 2001. 50; 609-613). Accordingly, one rationale for administering exogenous GLP-1, an incretin hormone, or an incretin mimetic, is to enhance, replace or supplement endogenous GLP-1 in order to increase meal-related insulin secretion, reduce glucagon secretion, and/or slow gastrointestinal motility. Native GLP-1 has a very short serum half-life (<5 minutes).

Albiglutide is marketed as TANZEUM™ in the United States and EPERZAN™ in Europe. The active ingredient of TANZEUM™/EPERZAN™ is albiglutide. Albiglutide is a recombinant fusion protein consisting of 2 copies of a 30-amino acid sequence of modified human glucagon-like peptide 1 (GLP-1, fragment 7-36(A8G)) genetically fused in series to recombinant human serum albumin. Each GLP-1 sequence has been modified with a glycine substituted for the naturally-occurring alanine at position 8 in order to confer resistance to dipeptidyl peptidase IV (DPP-IV)-mediated proteolysis. TANZEUM™/EPERZAN™ is provided as a 30 mg albiglutide pen for subcutaneous injection and contains 40.3 mg lyophilized albiglutide and 0.65 mL water for injection diluent designed to deliver a dose of 30 mg in a volume of 0.5 mL after reconstitution. TANZEUM™/EPERZAN™ is also provided as a 50 mg pen that contains 67 mg lyophilized albiglutide and 0.65 mL water for injection diluent designed to deliver a dose of 50 mg in a volume of 0.5 mL after reconstitution. Inactive ingredients include 153 mM mannitol, 0.01% (w/w) polysorbate 80, 10 mM sodium phosphate, 117 mM trehalose dihydrate. TANZEUM™/EPERZAN™ does not contain a preservative.

Lyophilized or freeze-dried preparations of a therapeutic protein such as albiglutide have the disadvantage of requiring complex packaging since a separate supply of sterile water for injection is required. Moreover, lyophilized preparations are typically administered using a dual-chambered injection cartridge. Dual-chamber cartridges can be expensive and may require upto 30 minutes to an hour after the water for injection is mixed with the lyophilized drug before the lyophilized active drug is reconstituted and ready for injection.

Thus, there is an unmet need for liquid compositions for hypoglycemic agents, in particular albiglutide or a variant thereof.

### BRIEF DESCRIPTION OF THE DRAWINGS

FIG. 1. Non-reduced SDS-PAGE gel for Study 1.0 formulations F07 through F12 at t0.

FIG. 2. Reduced SDS-PAGE gel for Study 1.0 formulations F07 through F12 at t0.

FIG. 3. Non-reduced and reduced SDS-PAGE gel for t2 samples from Study 1.0.

FIG. 4. Non-reduced SDS-PAGE gel for formulations F01-F07 from Study 1.1 at t0.

FIG. 5. Non-reduced SDS-PAGE gel for formulations F08-F14 from Study 1.1 at t0.

FIG. 6. Non-reduced SDS-PAGE gel for formulations F01-F07 from Study 1.1 at t1.

FIG. 7. Non-reduced SDS-PAGE gel for formulations F08-F14 from Study 1.1 at t1.

FIG. 8. Non-reduced SDS-PAGE gel for formulations F01-F07 from Study 1.1 at t2.

FIG. 9. Non-reduced SDS-PAGE gel for formulations F01-F07 from Study 1.1 at t2.

FIG. 10. Non-reduced SDS-PAGE gels for formulation F01 through F08 from Study 1.2 at t0.

FIG. 11. Non-reduced SDS-PAGE gels for formulation F09 through F16 from Study 1.2 at t0.

FIG. 12. Non-reduced SDS-PAGE gels for formulation F01 through F18 from Study 1.2 at t1.

FIG. 13. Non-reduced SDS-PAGE gels for formulation F09 through F16 from Study 1.2 at t1.

FIG. 14. Non-reduced SDS-PAGE gels for formulation F09 through F16 from Study 1.2 at t2.

FIG. 15. Non-reduced SDS-PAGE gels for formulation F09 through F16 from Study 1.2 at t2.

FIG. 16. Effect of pH and citrate according to the PLS1 model using the purity by RP HPLC at t22 at 25° C. as the endpoints. The protein concentration was fixed at 50 mg/mL, arginine at 100 mM, and trehalose at 117 mM.

FIG. 17. Effect of PS 80 and protein concentration according to the PLS1 model using the purity by RP HPLC at t22 at 25° C. as the endpoints. The citrate was fixed at 10 mM, the pH at 6.0, arginine at 100 mM, and trehalose at 117 mM.

FIG. 18. Effect of arginine and trehalose according to the PLS1 model using the purity by RP HPLC at t22 at 25° C. as the endpoints. The citrate was fixed at 10 mM, the pH at 6.0, and protein concentration at 50 mg/mL.

FIG. 19. Effect of pH and citrate according to the PLS2 model using the dimer content at t22 at 5° and 25° C. as the endpoints. The protein concentration was fixed at 50 mg/mL, Arg at 100 mM, and trehalose at 117 mM.

FIG. 20. Effect of PS 80 and protein concentration according to the PLS2 model using the dimer content at t22 at 5° and 25° C. as the endpoints. The citrate was fixed at 10 mM, the pH at 6.0, Arg at 100 mM, and trehalose at 117 mM.

FIG. 21. Effect of arginine and trehalose according to the PLS2 model using the dimer content at t22 at 5° and 25° C. as the endpoints. The citrate was fixed at 10 mM, the pH at 6.0, and protein concentration at 50 mg/mL.

FIG. 22. Effect of pH and citrate according to the PLS1 model using the main peak purity by cIEF at t22 at 5° as the endpoint. The protein concentration was fixed at 50 mg/mL, Arg at 100 mM, and trehalose at 117 mM.

FIG. 23. Effect of PS 80 and protein concentration according to the PLS1 model using the main peak purity by cIEF at t22 at 5° as the endpoint. The citrate was fixed at 10 mM, the pH at 6.0, Arg at 100 mM, and trehalose at 117 mM.

FIG. 24. Effect of arginine and trehalose according to the PLS1 model using the main peak purity by cIEF at t22 at 5° as the endpoint. The citrate was fixed at 10 mM, the pH at 6.0, and the protein concentration at 50 mg/mL.

### SUMMARY OF THE INVENTION

In one embodiment, the present invention provides liquid compositions comprising albiglutide, a buffering agent, at least one saccharide and/or at least one polyol, at least one stabilizing agent and optionally a surfactant wherein said albiglutide remains stable in said liquid composition. Albiglutide can be considered to remain stable in liquid if at least ≥96% of said albiglutide remains as a monomer in the liquid composition over a period of at least one week when protected from light.

In another embodiment, the present invention provides liquid compositions comprising a polypeptide having an amino acid sequence having at least 90%, 91%, 92%, 93%, 94%, 95%, 96%, 97%, 98%, 99% or 100% sequence identity to the polypeptide set forth in SEQ ID NO:1 or a polypeptide having at least 90%, 91%, 92%, 93%, 94%, 95%, 96%, 97%, 98%, 99% or 100% sequence identity to the polypeptide set forth in SEQ ID NO:1 which is truncated at the C-terminus and/or the N-terminus, at least one buffering agent, at least one saccharide and/or at least one polyol, and at least one stabilizing agent wherein said polypeptide remains stable and has at least one GLP-1 activity in said liquid composition. In one embodiment, the liquid composition further comprises a surfactant.

In another embodiment, the present invention provides a liquid composition comprising a GLP-1 agonist wherein said GLP-1 agonist remains stable and has at least one GLP-1 activity in said liquid composition for at least a year. Suitably, the GLP-1 agonist is albiglutide.

The present invention provides methods of treating a metabolic disorder, including type 2 diabetes comprising administering to a human any one of the liquid compositions of the present invention.

### DETAILED DESCRIPTION OF THE INVENTION

### Definitions

“GLP-1 agonist composition” as used herein means any composition capable of stimulating the secretion of insulin, or otherwise raising the level of insulin, including, but not limited to an incretin hormone and an incretin mimetic.

As used herein “GLP-1 agonist” and “GLP-1 receptor agonist” and GLP-1R agonist” are used interchangeably to refer to any compound capable of binding to GLP-1 receptor and/or having at least one GLP-1 activity. Suitably, albiglutide can be referred to as a GLP-1 agonist or a GLP-1R agonist.

“Incretin hormone” as used herein means any hormone that potentiates insulin secretion or otherwise raises the level of insulin in a mammal. One example of an incretin hormone is GLP-1. GLP-1 is an incretin hormone secreted by intestinal L cells in response to ingestion of food. In a healthy individual, GLP-1 plays an important role regulating post-prandial blood glucose levels by stimulating glucose-dependent insulin secretion by the pancreas resulting in increased glucose absorption in the periphery. GLP-1 also suppresses glucagon secretion, leading to reduced hepatic glucose output. In addition, GLP-1 delays gastric emptying time and slows small bowel motility delaying food absorption. GLP-1 promotes continued beta cell competence by stimulating transcription of genes involved in glucose dependent insulin secretion and by promoting beta-cell neogenesis (Meier, et al. Biodrugs 2003; 17 (2): 93-102).

“GLP-1 activity” as used herein means one or more of the activities of naturally occurring human GLP-1, including but not limited to, reducing blood and/or plasma glucose, stimulating glucose-dependent insulin secretion or otherwise raising the level or insulin, suppressing glucagon secretion, reducing fructosamine, increases glucose delivery and metabolism to the brain, delaying gastric emptying, and promoting beta cell competence, and/or neogenesis when administered to a mammal, including a human. Any of these activities and other activity associated with GLP-1 activity may be caused directly or indirectly by a composition having GLP-1 activity or a GLP-1 agonist. By way of example, a composition having GLP-1 activity may directly or indirectly stimulate insulin production which causes a reduction in plasma glucose levels in a mammal. As is understood in the art, GLP-1 activities can be measured by various standard means. GLP-1 activity can be measured in vivo and/or in vitro. By way of example, the GLP-1 activity of a polypeptide having the amino acid sequence set forth in SEQ ID NO:1 or a polypeptide having 90%, 91%, 92%, 93%, 94%, 95%, 96%, 97%, 98%, 99% or 100% sequence identity to SEQ ID NO:1 can be measured using standard techniques for measuring blood or plasma glucose levels before and after administration to a mammal. By way of another example, GLP-1 activity of a polypeptide having the amino acid sequence set forth in SEQ ID NO:1 or a polypeptide having 90%, 91%, 92%, 93%, 94%, 95%, 96%, 97%, 98%, 99% or 100% sequence identity to SEQ ID NO:1 can be measured in vitro.

The ability of GLP-1 to mediate its known cellular effects, including the induction of insulin secretion, is dependent upon its binding and subsequent activation of the GLP-1 receptor. This activation leads to an increase in intracellular 3′,5′-cyclic adenosine monophosphate (cAMP) production by adenylate cyclases that can be measured by ELISA. Albiglutide causes similar effects on sensitive cells, including induction of cAMP production. A bioassay using a HEK293F cell line engineered to stably express the human GLP-1 receptor can be used to determine the biological activity of the GLP-1 agonist in vitro. This assay is based upon the principle that binding of albiglutide to the GLP-1 receptor expressed on HEK293F cells causes an increase in intracellular cAMP levels that can be detected and quantified by ELISA. By comparing the effective concentration, 50% (EC₅₀) of albiglutide reference standard to test samples the relative potencies of those test samples may be determined.

GLP-1 agonists, including albiglutide, retain GLP-1 receptor binding activity relative to the native GLP-1 or albiglutide reformulated from lyophilized pellet. For example, the albiglutide in a liquid composition of the present invention can retain at least 10%, 20%, 30%, 40%, 50%, 60%, 70%, 75%, 80%, 85%, 90%, 95%, 96%, 97%, 98%, 99%, or 100% activity of the activity of native GLP-1 or albiglutide reconstituted from lyophilized pellet (calculated as the inverse ratio of EC50s for the albiglutide liquid composition vs. albiglutide reconstituted, e.g., as measured by cAMP production). In one embodiment, the albiglutide in liquid composition has the same, 100%, 99%, 98%, 97%, 96%, 95% or 90% activity (used synonymously with the term “potency” herein) compared with reconstituted albiglutide. Suitably, albiglutide in a liquid composition of the present invention can retain about 90% to 100% activity of the activity of native GLP-1 or albiglutide reconstituted from lyophilized pellet.

As used herein “potency” of a GLP-1 agonist, including but not limited to albiglutide, means the ability of the GLP-1 agonist to demonstrate at least one GLP-1 activity in vivo and/or in vitro to activate the GLP-1 receptor in a cell-based assay. As is described herein and as is understood in the art, potency of a GLP-1 agonist including albiglutide and variants thereof can be measured by several in vivo and in vitro methods. In some aspects of the present invention, the GLP-1 agonist in any of the liquid compositions may have between about 80% to about 130% of the potency of albiglutide. In some aspects, the GLP-1 agonist may have more than 130% of the potency of albiglutide. In some aspects, the potency of a GLP-1 agonist in a liquid formulation of the present invention can be measured against itself as a difference between Time 0 and T1. Additionally or alternatively, the potency of a GLP-1 agonist in a liquid formulation of the present invention, can be measured against the potency of albiglutide in the same formulation, under the same conditions and the same time points.

An “incretin mimetic” as used herein is a compound capable of potentiating insulin secretion or otherwise raise the level or insulin. An incretin mimetic may be capable of stimulating insulin secretion, increasing beta cell neogenesis, inhibiting beta cell apoptosis, inhibiting glucagon secretion, delaying gastric emptying and inducing satiety in a mammal. An incretin mimetic may include, but is not limited to, any polypeptide which has GLP-1 activity, including but not limited to, exendin 3 and exendin 4, including any fragments and/or variants and/or conjugates thereof.

“Hypoglycemic agent” as used herein means any compound or composition comprising a compound capable of reducing blood glucose. A hypoglycemic agent may include, but is not limited to, any GLP-1 agonist including incretin hormones or incretin mimetics, GLP-1 and/or fragment, variant and/or conjugate thereof. Other hypoglycemic agents include, but are not limited to, drugs that increase insulin secretion (e.g., sulfonylureas (SU) and meglitinides), inhibit GLP-1 break down (e.g., DPP-IV inhibitors), increase glucose utilization (e.g., glitazones, thiazolidinediones (TZDs) and/or pPAR agonists), reduce hepatic glucose production (e.g., metformin), and delay glucose absorption (e.g., α-glucosidase inhibitors). Examples of sulfonylureas include but are not limited to acetohexamide, chlorpropamide, tolazamide, glipizide, gliclazide, glibenclamide (glyburide), gliquidone, and glimepiride. Examples of glitazones include, but are not limited to, rosiglitazone and pioglitazone. “Polynucleotide(s)” generally refers to any polyribonucleotide or polydeoxyribonucleotide that may be unmodified RNA or DNA or modified RNA or DNA.

“Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that comprise one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques that are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, PROTEINS—STRUCTURE AND MOLECULAR PROPERTIES, 2nd Ed., T. E. Creighton, W.H. Freeman and Company, New York, 1993 and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter, et al., “Analysis for protein modifications and nonprotein cofactors”, Meth. Enzymol. (1990) 182:626-646 and Rattan, et al., “Protein Synthesis: Posttranslational Modifications and Aging”, Ann NY Acad Sci (1992) 663:48-62.

“Variant” as the term is used herein, is a polynucleotide or polypeptide that differs from a reference polynucleotide or polypeptide respectively, but retains essential properties such as retaining the biological activity of the reference polynucleotide or polypeptide. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis. Variants may also include, but are not limited to, polypeptides or fragments thereof having chemical modification of one or more of its amino acid side groups. A chemical modification includes, but is not limited to, adding chemical moieties, creating new bonds, and removing chemical moieties. Modifications at amino acid side groups include, without limitation, acylation of lysine-ε-amino groups, N-alkylation of arginine, histidine, or lysine, alkylation of glutamic or aspartic carboxylic acid groups, and deamidation of glutamine or asparagine. Modifications of the terminal amino group include, without limitation, the des-amino, N-lower alkyl, N-di-lower alkyl, and N-acyl modifications. Modifications of the terminal carboxy group include, without limitation, the amide, lower alkyl amide, dialkyl amide, and lower alkyl ester modifications. Furthermore, one or more side groups, or terminal groups, may be protected by protective groups known to the ordinarily-skilled protein chemist.

As used herein “fragment,” when used in reference to a polypeptide, is a polypeptide having an amino acid sequence that is the same as part but not all of the amino acid sequence of the entire naturally occurring polypeptide. Fragments may be “free-standing” or comprised within a larger polypeptide of which they form a part or region as a single continuous region in a single larger polypeptide. Thus, a fragment of GLP-1 may be “free-standing” or may be genetically fused and part of a larger amino acid sequence. By way of example, a fragment of naturally occurring GLP-1 would include amino acids 7 to 36 of naturally occurring amino acids 1 to 36. Furthermore, fragments of a polypeptide may also be variants of the naturally occurring partial sequence. For instance, a fragment of GLP-1 comprising amino acids 7-36 of naturally occurring GLP-1 may also be a variant having amino acid substitutions within its partial sequence, such as Ala to Gly at position 8.

As used herein “conjugate” or “conjugated” refers to two molecules that are bound to each other. For example, a first polypeptide may be covalently or non-covalently bound to a second polypeptide. The first polypeptide may be covalently bound by a chemical linker or may be genetically fused to the second polypeptide, wherein the first and second polypeptide share a common polypeptide backbone. Any of the GLP-1 polypeptides described herein can be bound by a linker to another stabilizing polypeptide such as albumin and/or a fragment thereof or human Fc region, such as a modified IgG4 or modified IgG1.

As used herein “tandemly oriented” refers to two or more polypeptides that are adjacent to one another as part of the same molecule. They may be linked either covalently or non-covalently. Two or more tandemly oriented polypeptides may form part of the same polypeptide backbone. Tandemly oriented polypeptides may have direct or inverted orientation and/or may be separated by other amino acid sequences.

As used herein “albiglutide” or “ALB” refer to a recombinant fusion protein consisting of 2 copies of a 30-amino acid sequence of modified human glucagon-like peptide 1 (GLP-1, fragment 7-36(A8G)) genetically fused in series to recombinant human serum albumin. The amino acid sequence of albiglutide is shown below as SEQ ID NO:1.

| (SEQ ID NO: 1) | |
|---|---|
| HGEGTFTSDVSSYLEGQAAKEFIAWLVKGRHGEGTFTSDVSSYLEGQAAKEFIAWLVKGR | 60 |
| DAHKSEVAHRFKDLGEENFKALVLIAFAQYLQQCPFEDHVKLVNEVTEFAKTCVADESAE | 120 |
| NCDKSLHTL FGDKLCTVATLRETYGEMADCCAKQEPERNECFLQHKDDNPNLPRLVRPE | 180 |
| VDVMCTAFHDNEETFLKKYLYEIARRHPYFYAPELLFFAKRYKAAFTECCQAADKAACLL | 240 |
| PKLDELRDEGKASSAKQRLKCASLQKFGERAFKAWAVARLSQRFPKAEFAEVSKLVTDLT | 300 |
| KVHTECCHGDLLECADDRADLAKYICENQDSISSKLKECCEKPLLEKSHCIAEVENDEMP | 360 |
| ADLPSLAADFVESKDVCKNYAEAKDVFLGMFLYEYARRHPDYSVVLLLRLAKTYETTLEK | 420 |
| CCAAADPHECYAKVFDEFKPLVEEPQNLIKQNCELFEQLGEYKFQNALLVRYTKKVPQVS | 480 |
| TPTLVEVSRNLGKVGSKCCKHPEAKRMPCAEDYLSVVLNQLCVLHEKTPVSDRVTKCCTE | 540 |
| SLVNRRPCFSALEVDETYVPKEFNAETFTFHADICTLSEKERQIKKQTALVELVKHKPKA | 600 |
| TKEQLKAVMDDFAAFVEKCCKADDKETCFAEEGKKLVAASQAALGL | 674 |

As used herein, “reduce” or “reducing” blood or plasma glucose refers to a decrease in the amount of blood glucose observed in the blood or plasma of a patient after administration a hypoglycemic agent. Reductions in blood or plasma glucose can be measured and assessed per individual or as a mean change for a group of subjects. Additionally, mean reductions in blood or plasma glucose can be measured and assessed for a group of treated subjects as a mean change from baseline and/or as a mean change compared with the mean change in blood or plasma glucose among subjects administered placebo.

As used herein “enhancing GLP-1 activity” refers to an increase in any and all of the activities associated with naturally occurring GLP-1. By way of example, enhancing GLP-1 activity can be measured after administration of at least one polypeptide having GLP-1 activity to a subject and compared with GLP-1 activity in the same subject prior to the administration of the polypeptide having GLP-1 activity or in comparison to a second subject who is administered placebo.

As used herein “diseases associated with elevated blood glucose” include, but are not limited to, type 1 and type 2 diabetes, glucose intolerance, and hyperglycemia.

As used herein “co-administration” or “co-administering” refers to administration of two or more compounds or two or more doses of the same compound to the same patient. Co-administration of such compounds may be simultaneous or at about the same time (e.g., within the same hour) or it may be within several hours or days of one another. For example, a first compound may be administered once weekly while a second compound is co-administered daily.

As used herein “maximum plasma concentration” or “Cmax” means the highest observed concentration of a substance (for example, a polypeptide having GLP-1 activity or a GLP-1 agonist) in mammalian plasma after administration of the substance to the mammal.

As used herein “Area Under the Curve” or “AUC” is the area under the curve in a plot of the concentration of a substance in plasma against time. AUC can be a measure of the integral of the instantaneous concentrations during a time interval and has the units mass×time/volume, which can also be expressed as molar concentration×time such as nM×day. AUC is typically calculated by the trapezoidal method (e.g., linear, linear-log). AUC is usually given for the time interval zero to infinity, and other time intervals are indicated (for example AUC (t1,t2) where t1 and t2 are the starting and finishing times for the interval). Thus, as used herein “AUC_{0-24 h}” refers to an AUC over a 24-hour period, and “AUC_{0-4 h}” refers to an AUC over a 4-hour period.

As used herein “weighted mean AUC” is the AUC divided by the time interval over which the time AUC is calculated. For instance, weighted mean AUC_{0-24 h} would represent the AUC_{0-24 h} divided by 24 hours.

As used herein “confidence interval” or “CI” is an interval in which a measurement or trial falls corresponding to a given probability p where p refers to a 90% or 95% CI and are calculated around either an arithmetic mean, a geometric mean, or a least squares mean. As used herein, a geometric mean is the mean of the natural log-transformed values back-transformed through exponentiation, and the least squares mean may or may not be a geometric mean as well but is derived from the analysis of variance (ANOVA) model using fixed effects.

As used herein the “coefficient of variation (CV)” is a measure of dispersion and it is defined as the ratio of the standard deviation to the mean. It is reported as a percentage (%) by multiplying the above calculation by 100 (% CV).

As used herein “dose” refers to any amount of therapeutic compound which may be administered to a mammal, including a human. An effective dose is a dose of a compound that is in an amount sufficient to induce at least one of the intended effects of the therapeutic compound. For instance, an effective dose of a GLP-1 agonist would induce at least one type of GLP-1 activity in a human when administered to a human, such as, but not limited to increasing insulin production in said human. As is understood in the art, an effective dose of a therapeutic compound can be measured by a surrogate endpoint. Thus, by way of another example, an effective dose of a GLP-1 agonist can be measured by its ability to lower serum glucose in a human.

As used herein “a buffering agent” or “buffer” and grammatical variations thereof means any component of a solution that can act to maintain the pH of a liquid compositions. Suitably, the buffering agent or buffer can be any pharmaceutically acceptable buffer for injection. Examples of buffering agents, include, but are not limited to, citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium gluconate mixture, gluconic acid-sodium hydroxide mixture, gluconic acid-potassium gluconate mixture, etc.), oxalate buffers (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) phosphate buffers (sodium phosphate monobasic/sodium phosphate dibasic) and acetate buffers (e.g., acetic acid-sodium acetate mixture, acetic acid-sodium hydroxide mixture, etc.). Additionally, histidine or histidine HCl and glycerine can be used as buffering agents. Suitably, the buffer will maintain the liquid composition within the desired pH range of about 5.7 to about 6.2, or about 5.8 to about 6.1, or about 5.9 to about 6.0. Suitably, the buffer will maintain the pH of the aqueous liquid compositions of the invention at about 5.9. The concentrations of a buffering agent will need to be adjusted to maintain the pH of the liquid compositions of the present invention. Suitably, the buffer is selected from sodium phosphate, sodium citrate, sodium succinate, sodium carbonate and sodium acetate.

As used herein a “saccharide” means a stabilizing sugar that is pharmaceutically acceptable for injection. Suitably, disaccharides include sucrose, lactulose, lactose, maltose, trehalose, raffinose, or cellobiose, and/or mixtures thereof. Other contemplated disaccharides include kojibiose, nigerose, isomaltose, ββ-trehalose, αβ-trehalose, sophorose, laminaribiose, gentiobiose, turanose, maltulose, palatinose, gentiobiulose, mannobiose, melibiose, melibiulose, rutinose, rutinulose, and xylobiose. A saccharide includes, but is not limited to, a disaccharide, monosaccharide or polysaccharide. The term “sugar” can be used to refer to all saccharides. A disaccharide can be, for example, sucrose or trehalose, or a mixture thereof. Suitably, a saccharide or a sugar can also serve as a stabilizing agent in the liquid compositions of the present invention. In some aspects of the present invention, the trehalose is trehalose dihydrate. In other aspects, the trehalose is trehalose monohydrate.

As used herein the term “polyol” refers to any sugar alcohol. Examples of polyols include but are not limited to, mannitol, maltitol, sorbitol, xylitol, erythritol, and isomalt. Sugar alcohols may be formed under mild reducing conditions from their analogue sugars. Suitably, a polyol can also serve as a stabilizing agent in the liquid compositions of the present invention.

The term “excipient” refers to any compound added during processing and/or storage to a liquid composition for the purpose of altering the bulk properties, improving stability and/or adjustment of osmolality.

The term “stabilizing agent” or “stabilizer” refers to an excipient that improves or otherwise enhances stability of an active ingredient, such as, but not limited to albiglutide. As used herein “stabilizing agent” or “stabilizer” refers to any agent that is capable of preventing the aggregation of the GLP-1 polypeptides, such as albiglutide, in the liquid compositions of the present invention. Stabilizing agents including, but are not limited to, arginine HCl and histidine HCl. Suitably, sodium octanoate can also act as a stabilizer. The term “stabilizing agent” is intended to encompass substances, or a mixture of substances, that are able to stabilize a polypeptide during storage or production of a composition comprising the polypeptide. Additionally, saccharides and sugars in general, polyols in general, trehalose, mannitol, sodium octanoate, citrate, succinate, and surfactants, including but not limited to, polysorbate 80, can act as stabilizers. For instance, arginine and trehalose can both act as stabilizers in the same liquid composition also referred to as concert stabilizers.

The term “stability” or “stable” and grammatical variants thereof means the relative temporal constancy of at least one GLP-1 activity of albiglutide or variants thereof. The term “stabilizing” is intended to encompass minimizing the formation of aggregates (insoluble and/or soluble) and/or chemical degradation and/or protein unfolding of the polypeptide during storage or production of the compositions so that substantial retention of biological activity and polypeptide stability is maintained.

The term “physical stability” of GLP-1 polypeptides relates to the formation of insoluble and/or soluble aggregates in the form of dimeric, oligomeric and polymeric forms of GLP-1 polypeptides as well as any structural deformation and denaturation of the molecule.

The term “chemical stability” is intended to relate to the formation of any chemical change in the GLP-1 polypeptides upon storage in dissolved or solid state at accelerated conditions. By example are hydrolysis, deamidation and oxidation. In particular, the sulphur-containing amino acids are prone to oxidation with the formation of the corresponding sulphoxides. The term “chemical stability” also includes resistance to chemical degradation of the polypeptide backbone, for instance, by proteolysis.

Excipients for stabilizing proteins in general are described in Manning et al., “Stability of protein pharmaceuticals,” Pharmaceutical Research, 6:903-918 (1989); Wang et al., “Review of excipients and pH's for parenteral products used in the United States,” Journal of Parenteral Drug Association, 34:452-462 (1980); Wang et al., “Parenteral formulations of proteins and peptides: stability and stabilizers,” Journal of Parenteral Science & Technology, 42:S4-S26 (1988); Cleland et al., “The Development of Stable Protein Formulations: A Close Look at Protein Aggregation, Deamidation, and Oxidation”, Critical Reviews in Therapeutic Drug Carrier Systems, vol. 10(4), pp. 307-377 (1993).

As used herein a “surfactant” refers to any component that lowers the surface tension of the liquid composition. Surfactants can be ionic or non-ionic. Suitable surfactants for injections include, but are not limited to, polysorbate 80, polysorbate 20, and Pluronic F68. Suitably, surfactants can also serve as a stabilizing agent in the liquid compositions of the present invention.

The present invention provides liquid compositions comprising albiglutide, a buffering agent, at least one saccharide and/or at least one polyol, at least one stabilizing agent and optionally a surfactant wherein said albiglutide remains stable in said liquid composition. In one embodiment, the buffering agent comprises sodium citrate in any of the liquid compositions of the present invention. In one embodiment, the buffering agent consists of sodium citrate. The sodium citrate suitably is at a concentration of about 5 mM to about 15 mM citrate in any of the liquid compositions of the present invention. The sodium citrate is at a concentration of about 10 mM citrate in any of the liquid compositions of the present invention. In one aspect, the pH of the liquid composition is between about 5.5 to about 5.7 if the citrate concentration is above 10 mM. In another aspect, the pH is about 5.9 if the citrate concentration is 10 mM citrate in any of the liquid compositions of the present invention.

In one aspect, the buffering agent comprises succinate citrate in any of the liquid compositions of the present invention. In one embodiment, the buffering agent consists of succinate. The succinate may be at a concentration of about 5 mM to about 10 mM in any of the liquid compositions of the present invention.

In one embodiment, the liquid compositions of the present invention comprise a saccharide. In one embodiment the saccharide comprises trehalose. In one embodiment, the saccharide consists of trehalose. The trehalose can be at a concentration of about 72 mM to about 207 mM in any of the liquid compositions of the present invention. In some aspects trehalose can be at a concentration of about 75 mM, 80 mM, 85 mM, 90 mM, 95 mM, 100 mM, 105 mM, 110 mM, 115 mM, 120 mM, or 125 mM. Suitably, trehalose can be at a concentration in the range of about 100 mM to about 140 mM or about 100 mM to about 120 mM in the liquid composition. In one aspect, trehalose is at a concentration of about 117 mM in said liquid composition. In one embodiment, the liquid composition comprises a saccharide but does not comprise a polyol. In one embodiment, the liquid composition comprises a polyol and does not comprise a saccharide. In one embodiment, the liquid composition comprises both a saccharide and a polyol.

In one embodiment, the stabilizing agent comprises arginine in any of the liquid compositions of the present invention. In one aspect, the stabilizing agent is arginine. Suitably, the arginine is at a concentration of about 50 mM to about 125 mM in any of the liquid compositions of the present invention. In one aspect, the arginine can be at a concentration of about 45 mM, 50 mM, 55 mM, 60 mM, 70 mM, 75 mM, 80 mM, 85 mM, 90 mM, 95 mM, 100 mM, 105 mM, 110 mM, 115 mM, 120 mM, 125 mM or 130 mM. Suitably, the arginine can be at a concentration in the range of about 90 mM to about 110 mM in the liquid composition. Suitably, the arginine concentration could range from about 80 mM to 120 mM in the liquid composition. The arginine may be at a concentration of about 100 mM in any of the liquid compositions of the present invention.

In another aspect the stabilizing agent comprises histidine. In one aspect, the histidine is at a concentration of about 50 mM to about 125 mM in any of the liquid compositions of the present invention. Suitably, the histidine can be at a concentration in the range of about 90 mM to about 110 mM in the liquid composition. Suitably, the histidine concentration could range from about 80 mM to 120 mM in any of the liquid compositions of the present invention. In one aspect, the histidine is at a concentration of about 100 mM in any of the liquid compositions of the present invention.

In one embodiment, the liquid composition comprises a surfactant. The surfactant is polysorbate 80. The polysorbate 80 is about 0.005% w/w to about 0.02% w/w in any of the liquid compositions of the present invention. The polysorbate 80 is at a concentration of 0.01% w/w in any of the liquid compositions of the present invention.

In one embodiment, the liquid compositions of the present invention further comprise methionine and/or EDTA. Suitably, the liquid compositions of the present invention comprise water for injection.

In one embodiment the liquid composition comprises a polyol. In one embodiment the polyol comprises mannitol. In one embodiment, the polyol consists of mannitol. In one embodiment, the liquid composition comprises a polyol but does not comprise a saccharide.

Suitably, albiglutide is present at a concentration of about 30 mg/mL to about 100 mg/mL in any of the liquid compositions of the present invention. Albiglutide is present at a concentration of about 60 mg/mL in any of the liquid compositions of the present invention. Albiglutide is present at a concentration of about 50 mg/mL in any of the liquid compositions of the present invention. Suitably, albiglutide can be present at about 30 mg/mL, 35 mg/mL, 40 mg/mL, 45 mg/mL, 50 mg/mL, 55 mg/mL, 60 mg/mL or 65 mg/mL. Suitably, albiglutide can be present at about 40 mg/mL to about 80 mg/mL or about 50 mg/mL to about 70 mg/mL. In some aspects, albiglutide is heat-treated after purification and prior to formulation.

In one aspect, the liquid compositions of the invention have a pH between about 5.0 and 6.5. Suitably, the pH of the liquid compositions can range from about 5.5 to about 6.2. Suitably, the pH of the liquid composition is about 5.5 to about 5.9. In one aspect the pH is about 5.5 to about 5.7. In one aspect, the liquid compositions have a pH of about 5.9.

In one embodiment, at least ≥90%. ≥95%, ≥96%, ≥97%, suitably ≥98%, suitably ≥99%, and suitably 100% of albiglutide in the liquid compositions of the present invention remains as a monomer in any of the liquid composition of the present invention. Stability of albiglutide as a monomer can be measured by various techniques known and the art and is described in the examples provided herein. As is described herein, albiglutide will remain as a monomer in the liquid composition of the present invention for up to one week when stored at room temperature and up to 12 months, 18 months and 24 months when stored at about 2° C. to about 8° C. when protected from light. In another aspect, albiglutide in said liquid composition has at least one GLP-1 activity and maintains said at least one GLP-1 activity at at least 90% potency for at least 12 months. In one embodiment the liquid formulations of the present invention are protected from light.

In one embodiment, the present invention provides liquid compositions comprising a polypeptide having at least 90%, 91%, 92%, 93%, 94%, 95%, 96%, 97%, 98%, 99% or 100% sequence identity to the polypeptide set forth in SEQ ID NO:1 or a polypeptide having at least 90%, 91%, 92%, 93%, 94%, 95%, 96%, 97%, 98%, 99% or 100% sequence identity to the polypeptide set forth in SEQ ID NO:1 which is truncated at the C-terminus and/or the N-terminus, at least one buffering agent, at least one saccharide and/or at least one polyol, at least one stabilizing agent and optionally at least one surfactant wherein said polypeptide remains stable in said liquid composition. In one aspect, the polypeptide is truncated at the N-terminus by 1, 2, 3, 4, 5, 6, 7, 8, 9, or 10 amino acids compared to SEQ ID NO:1 or a polypeptide having at least 90%, 91%, 92%, 93%, 94%, 95%, 96%, 97%, 98%, 99% or 100% sequence identity to SEQ ID NO:1 over the entire sequence. In one aspect, the polypeptide is truncated at the C-terminus by 1, 2, 3, 4, 5, 6, 7, 8, 9, or 10 amino acids compared to SEQ ID NO:1 or a polypeptide having at least 90%, 91%, 92%, 93%, 94%, 95%, 96%, 97%, 98%, 99% or 100% sequence identity to SEQ ID NO:1 over the entire sequence.

In one aspect the polypeptide has 100% sequence identity to the polypeptide set forth in SEQ ID NO:1. In one aspect, the liquid composition comprises a surfactant. In one aspect, the liquid composition comprises sodium citrate, trehalose, arginine, polysorbate 80 and water wherein said composition has a pH of about 5.9. In one aspect, the polypeptide is in said liquid composition at a concentration of about 30 mg/mL to about 100 mg/mL, suitably the concentration of said polypeptide is about 60 mg/mL, suitably the concentration of said polypeptide is about 50 mg/mL.

In one embodiment, the present invention provides liquid compositions comprising about 30 mg/mL to about 100 mg/mL of a polypeptide having at least 90%, 91%, 92%, 93%, 94%, 95%, 96%, 97%, 98%, 99% or 100% sequence identity to the polypeptide set forth in SEQ ID NO:1 or a polypeptide having at least 90%, 91%, 92%, 93%, 94%, 95%, 96%, 97%, 98%, 99% or 100% sequence identity to the polypeptide set forth in SEQ ID NO:1 which is truncated at the C-terminus and/or the N-terminus, about 110 mM to about 140 mM trehalose, 90 mM to about 110 mM arginine, 5 mM to about 15 mM sodium citrate, and 0.01% w/w polysorbate 80 wherein said composition has a pH of about 5.5 to about 6.0. In one embodiment the liquid composition comprises about 50 mg/mL of a polypeptide having the amino acid sequence set forth in SEQ ID NO:1. In one embodiment, the liquid composition consists of 50 mg/mL of a polypeptide having the amino acid sequence set forth in SEQ ID NO:1, 117 mM trehalose, 100 mM arginine, 10 mM sodium citrate, 0.01% w/w polysorbate 80 and water for injection wherein said composition has a pH of about 5.9.

The present invention also provides liquid compositions wherein the liquid compositions comprise a polypeptide that has at least 90% sequence identity to SEQ ID NO:1 and wherein said polypeptide has at least one GLP-1 activity and maintains said GLP-1 activity in said liquid composition for at least one week. In one aspect, at least 96% of said polypeptide remains as a monomer in any of the liquid composition of the present invention for at least a week. In one aspect, the polypeptide maintains said at least one GLP-1 activity at at least 90% potency in any of the liquid compositions of the present invention for at least 12 months. In one embodiment, the polypeptide remains stable in any of the liquid compositions of the present invention for at least 12 months when the composition is maintained at about 2° C. to about 8° C. when protected from light.

The present invention also provides liquid composition comprising a GLP-1 agonist wherein said GLP-1 agonist remains stable and has at least one GLP-1 activity in any of the liquid compositions of the present invention for at least a year. The liquid compositions can further comprise sodium citrate, trehalose, arginine, polysorbate 80 and water.

Also provided are liquid compositions comprising about 30 mg/mL to about 100 mg/mL of a polypeptide having the amino acid sequence set forth in SEQ ID NO:1, 117 mM trehalose, 100 mM arginine, 10 mM sodium citrate, and 0.01% w/w polysorbate 80 wherein said composition has a pH of about 5.9. Suitably, said liquid composition comprises about 60 mg/mL of a polypeptide having the amino acid sequence set forth in SEQ ID NO:1. Suitably, the liquid compositions of the present invention comprise about 50 mg/mL of a polypeptide having the amino acid sequence set forth in SEQ ID NO:1.

In one embodiment a liquid composition is provided consisting of 50 mg/mL of a polypeptide having the amino acid sequence set forth in SEQ ID NO:1, 117 mM trehalose, 100 mM arginine, 10 mM sodium citrate, 0.01% w/w polysorbate 80 and water for injection wherein said composition has a pH of about 5.9.

In one embodiment a liquid composition is provided consisting of 50-70 mg/mL of a polypeptide having the amino acid sequence set forth in SEQ ID NO:1, 100-140 mM trehalose, 90-110 mM arginine, 9-11 mM sodium citrate, 0.01% w/w polysorbate 80 and water for injection wherein said composition has a pH of about 5.5 to about 6.0.

In one embodiment, the liquid composition comprises a saccharide but does not comprise a polyol. In one embodiment of the present invention, the saccharide of the liquid composition is replaced by a polyol. Suitable examples include but are not limited to mannitol maltitol, sorbitol, xylitol, erythritol, and isomalt. In one embodiment, the liquid composition comprises mannitol.

In certain embodiments, methods are provided for treating a metabolic disorder in a human comprising administering a liquid composition to a human of the liquid compositions of the present invention. The metabolic disorder is type 2 diabetes mellitus (T2DM). In some aspects the liquid composition is administered to a human subcutaneously via a pen injection device. In one embodiment, the liquid composition is contained in a prefilled syringe, the liquid composition may be administered using an autoinjector device. In some aspects the autoinjector device has a gauge of 28, 29, 30 or greater, indicating a smaller aperture. In another embodiment, the device is an auto-injector. In one aspect the device has a 29 gauge (G) needle. In one aspect, the liquid compositions are used to provide glycemic control in humans in need thereof. Suitably, methods are provided for providing glycemic control in a human having T2DM comprising administering any of the liquid compositions of the present invention to said human.

The present invention also provides liquid compositions for use in treating metabolic disorders, for example diseases associated with elevated blood glucose include, but are not limited to, type 1 and type 2 diabetes, glucose intolerance, and hyperglycemia.

In a further embodiment the present invention provides liquid compositions of the present invention for use in manufacture of a medicament for treating metabolic disorders, for example diseases associated with elevated blood glucose including type 1 and type 2 diabetes, glucose intolerance, and hyperglycemia.

An embodiment of the invention comprises a polypeptide that may be, but is not limited to, GLP-1 or a fragment, variant, and/or conjugate thereof. GLP-1 fragments and/or variants and/or conjugates of the present invention typically have at least one GLP-1 activity. A GLP-1 or a fragment, variant, and/or conjugate thereof may comprise human serum albumin. Human serum albumin may be conjugated to the GLP-1 or fragment and/or variant thereof. Human serum albumin may be conjugated to an incretin hormone (such as GLP-1) and variants thereof through a chemical linker prior to injection or may be chemically linked to naturally occurring human serum albumin in vivo (see for instance, U.S. Pat. Nos. 6,593,295 and 6,329,336, herein incorporated by reference in their entirety). Alternatively, human serum albumin may be genetically fused to a GLP-1 and/or fragment and/or variant thereof or other GLP-1 agonist such as exendin-3 or exendin-4 and/or fragments and/or variants thereof. Examples of GLP-1 and fragments and/or variants thereof fused with human serum albumin are provided in the following: WO 2003/060071, WO 2003/59934, WO 2005/003296, WO 2005/077042 and U.S. Pat. No. 7,141,547 (herein incorporated by reference in their entirety).

Polypeptides having GLP-1 activity may comprise at least one fragment and/or variant of human GLP-1. The two naturally occurring fragments of human GLP-1 are represented in SEQ ID NO:2.

(SEQ ID NO.: 2)
7 8 9 10 11 12 13 14 15 16 17
His-Ala-Glu-Gly-Thr-Phe-Thr-Ser-Asp-Val-Ser-
18 19 20 21 22 23 24 25 26 27 28
Ser-Tyr-Leu-Glu-Gly-Gln-Ala-Ala-Lys-Glu-Phe-
29 30 31 32 33 34 35 36 37
Ile-Ala-Trp-Leu-Val-Lys-Gly-Arg-Xaa

wherein: Xaa at position 37 is Gly (hereinafter designated as “GLP-1(7-37)”), or —NH₂ (hereinafter designated as “GLP-1(7-36)”). GLP-1 fragments may include, but are not limited to, molecules of GLP-1 comprising, or alternatively consisting of, amino acids 7 to 36 of human GLP-1 (GLP-1(7-36)). Variants of GLP-1 or fragments thereof may include, but are not limited to, one, two, three, four, five or more amino acid substitutions in wild type GLP-1 or in the naturally occurring fragments of GLP-1 shown in SEQ ID NO:2. Variants GLP-1 or fragments of GLP-1 may include, but are not limited to, substitutions of an alanine residue analogous to alanine 8 of wild type GLP-1, such alanine being mutated to a glycine (hereinafter designated as “A8G”) (See for example, the mutants disclosed in U.S. Pat. No. 5,545,618, herein incorporated by reference in its entirety).

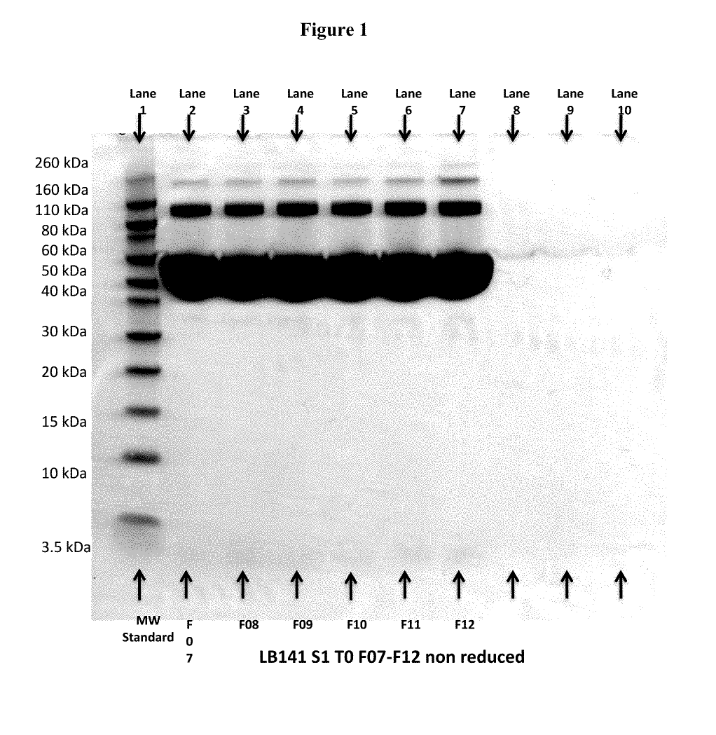

In some aspects, at least one fragment and variant of GLP-1 comprises GLP-1(7-36(A8G)) and is genetically fused to human serum albumin. In a further embodiment, polypeptides of the invention comprise one, two, three, four, five, or more tandemly oriented molecules of GLP-1 and/or fragments and/or variants thereof fused to the N- or C-terminus of human serum albumin or variant thereof. Other embodiments have such A8G polypeptides fused to the N- or C-terminus of albumin or variant thereof Δn example of two tandemly oriented GLP-1(7-36)(A8G) fragments and/or variants fused to the N-terminus of human serum albumin comprises SEQ ID NO:1, which is presented in FIG. 1. In another aspect, at least one fragment and variant of GLP-1 comprises at least two GLP-1(7-36(A8G)) tandemly and genetically fused to the human serum albumin. At least two GLP-1(7-36(A8G)) may be genetically fused at the N-terminus of the human serum albumin. At least one polypeptide having GLP-1 activity may comprise SEQ ID NO:1.

Variants of GLP-1(7-37) may be denoted for example as Glu²²-GLP-1(7-37)OH which designates a GLP-1 variant in which the glycine normally found at position 22 of GLP-1(7-37)OH has been replaced with glutamic acid; Val⁸-Glu²²-GLP-1(7-37)OH designates a GLP-1 compound in which alanine normally found at position 8 and glycine normally found at position 22 of GLP-1(7-37)OH have been replaced with valine and glutamic acid, respectively. Examples of variants of GLP-1 include, but are not limited to,

| | | |
|---|---|---|
| Val⁸-GLP-1(7-37)OH | Gly⁸-GLP-1(7-37)OH | Glu²²-GLP-1(7-37)O-H |
| Asp²²-GLP-1(7-37)OH | Arg²²-GLP-1(7-37)OH | Lys²²-GLP-1(7-37)OH |
| Cys²²-GLP-1(7-37)OH | Val⁸-Glu²²-GLP-1(7-37)OH | Val⁸-Asp²²-GLP-1(7-37)OH |
| Val⁸-Arg²²-GLP-1(7-37)OH | Val⁸-Lys²²-GLP-1(7-37)OH | Val⁸-Cys²²-GLP-1(7-37)OH |
| Gly⁸-Glu²²-GLP-1(7-37)OH | Gly⁸-Asp²²-GLP-1(7-37)OH | Gly⁸-Arg²²-GLP-1(7-37)OH |
| Gly⁸-Lys²²-GLP-1(7-37)OH | Gly⁸-Cys²²-GLP-1(7-37)OH | Glu²²-GLP-1(7-36)OH |
| Asp²²-GLP-1(7-36)OH | Arg²²-GLP-1(7-36)OH | Lys²²-GLP-1(7-36)OH |
| Cys²²-GLP-1(7-36)OH | Val⁸-Glu²²-GLP-1(7-36)OH | Val⁸-Asp²²-GLP-1(7-36)OH |
| Val⁸-Arg²²-GLP-1(7-36)OH | Val⁸-Lys²²-GLP-1(7-36)OH | Val⁸-Cys²²-GLP-1(7-36)OH |
| Gly⁸-Glu²²-GLP-1(7-36)OH | Gly⁸-Asp²²-GLP-1(7-36)OH | Gly⁸-Arg²²-GLP-1(7-36)OH |
| Gly⁸-Lys²²-GLP-1(7-36)OH | Gly⁸-Cys²²-GLP-1(7-36)OH | Lys²³-GLP-1(7-37)OH |
| Val⁸-Lys²³-GLP-1(7-37)OH | Gly⁸-Lys²³-GLP-1(7-37)OH | His²⁴-GLP-1(7-37)OH |
| Val⁸-His²⁴-GLP-1(7-37)OH | Gly⁸-His²⁴-GLP-1(7-37)OH | Lys²⁴-GLP-1(7-37)OH |
| Val⁸-Lys²⁴-GLP-1(7-37)OH | Gly⁸-Lys²³-GLP-1(7-37)OH | Glu³⁰-GLP-1(7-37)OH |
| Val⁸-Glu³⁰-GLP-1(7-37)OH | Gly⁸-Glu³⁰-GLP-1(7-37)OH | Asp³⁰-GLP-1(7-37)OH |
| Val⁸-Asp³⁰-GLP-1(7-37)OH | Gly⁸-Asp³⁰-GLP-1(7-37)OH | Gln³⁰-GLP-1(7-37)OH |
| Val⁸-Gln³⁰-GLP-1(7-37)OH | Gly⁸-Gln³⁰-GLP-1(7-37)OH | Tyr³⁰-GLP-1(7-37)OH |
| Val⁸-Tyr³⁰-GLP-1(7-37)OH | Gly⁸-Tyr³⁰-GLP-1(7-37)OH | Ser³⁰-GLP-1(7-37)OH |
| Val⁸-Ser³⁰-GLP-1(7-37)OH | Gly⁸-Ser³⁰-GLP-1(7-37)OH | His³⁰-GLP-1(7-37)OH |
| Val⁸-His³⁰-GLP-1(7-37)OH | Gly⁸-His³⁰-GLP-1(7-37)OH | Glu³⁴-GLP-1(7-37)OH |
| Val⁸-Glu³⁴-GLP-1(7-37)OH | Gly⁸-Glu³⁴-GLP-1(7-37)OH | Ala³⁴-GLP-1(7-37)OH |
| Val⁸-Ala³⁴-GLP-1(7-37)OH | Gly⁸-Ala³⁴-GLP-1(7-37)OH | Gly³⁴-GLP-1(7-37)OH |
| Val⁸-Gly³⁴-GLP-1(7-37)OH | Gly⁸-Gly³⁴-GLP-1(7-37)OH | Ala³⁵-GLP-1(7-37)OH |
| Val⁸-Ala³⁵-GLP-1(7-37)OH | Gly⁸-Ala³⁵-GLP-1(7-37)OH | Lys³⁵-GLP-1(7-37)OH |
| Val⁸-Lys³⁵-GLP-1(7-37)OH | Gly⁸-Lys³⁵-GLP-1(7-37)OH | His³⁵-GLP-1(7-37)OH |
| Val⁸-His³⁵-GLP-1(7-37)OH | Gly⁸-His³⁵-GLP-1(7-37)OH | Pro³⁵-GLP-1(7-37)OH |
| Val⁸-Pro³⁵-GLP-1(7-37)OH | Gly⁸-Pro³⁵-GLP-1(7-37)OH | Glu³⁵-GLP-1(7-37)OH |
| Gly⁸-Glu³⁵-GLP-1(7-37)OH | Val⁸-Ala²⁷-GLP-1(7-37)OH | Val⁸-His³⁷-GLP-1(7-37)OH |
| Val⁸-Glu²²-Lys²³-GLP-1(7-37)OH | Val⁸-Glu²²-Glu²³-GLP-1(7-37)OH | Val⁸-Glu²²-Ala²⁷-GLP-1(7-37)OH |
| Val⁸-Gly³⁴-Lys³⁵-GLP-1(7-37)OH | Val⁸-His³⁷-GLP-1-(7-37)OH | Gly⁸-His³⁷-GLP-1(7-37)OH |
| Val⁸-Glu²²-Ala²⁷-GLP-1(7-37)OH | Gly⁸-Glu²²-Ala²⁷-GLP-1(7-37)OH | Val⁸-Lys²²-Glu²³-GLP-1(7-37)OH |
| Gly⁸-Lys²²-Glu²³-GLP-1(7-37)OH | Val⁸-Glu³⁵-GLP-1(7-37)OH | |

Variants of GLP-1 may also include, but are not limited to, GLP-1 or GLP-1 fragments having chemical modification of one or more of its amino acid side groups. A chemical modification includes, but is not limited to, adding chemical moieties, creating new bonds, and removing chemical moieties. Modifications at amino acid side groups include, without limitation, acylation of lysine-ε-amino groups, N-alkylation of arginine, histidine, or lysine, alkylation of glutamic or aspartic carboxylic acid groups, and deamidation of glutamine or asparagine. Modifications of the terminal amino group include, without limitation, the des-amino, N-lower alkyl, N-di-lower alkyl, and N-acyl modifications. Modifications of the terminal carboxy group include, without limitation, the amide, lower alkyl amide, dialkyl amide, and lower alkyl ester modifications. Furthermore, one or more side groups, or terminal groups, may be protected by protective groups known to the ordinarily-skilled protein chemist.

GLP-1 fragments or variants may also include polypeptides in which one or more amino acids have been added to the N-terminus and/or C-terminus of GLP-1(7-37)OH of said fragment or variant. The amino acids in GLP-1 in which amino acids have been added to the N-terminus or C-terminus are denoted by the same number as the corresponding amino acid in GLP-1(7-37)OH. For example, the N-terminus amino acid of a GLP-1 compound obtained by adding two amino acids to the N-terminus of GLP-1(7-37)OH is at position 5; and the C-terminus amino acid of a GLP-1 compound obtained by adding one amino acid to the C-terminus of GLP-1(7-37)OH is at position 38. Thus, position 12 is occupied by phenylalanine and position 22 is occupied by glycine in both of these GLP-1 compounds, as in GLP-1(7-37)OH. Amino acids 1-6 of a GLP-1 with amino acids added to the N-terminus may be the same as or a conservative substitution of the amino acid at the corresponding position of GLP-1(1-37)OH. Amino acids 38-45 of a GLP-1 with amino acids added to the C-terminus may be the same as or a conservative substitution of the amino acid at the corresponding position of glucagon or exendin-4.

Albiglutide is provided commercially in a lyophilized form using a dual cartridge device for reconstituting and injection. The present invention provides liquid formulations of albiglutide which are stable in liquid form. The following table compares the lyophilized form with a stable liquid formulation.

| Commercial Lyo Product | Liquid formulation |
|---|---|
| 30 mg dose strength: 60 mg/mL albiglutide | 50 mg/mL albiglutide |
| 50 mg dose strength: 100 mg/mL albiglutide | For 30 mg and 50 mg dose strengths |
| 10 mM sodium phosphate, 117 mM trehalose, | 10 mM sodium citrate, 117 mM trehalose, 100 mM |
| 153 mM mannitol, 0.01% (w/w) polysorbate | arginine, 0.01% (w/w) polysothate 80, pH 5.9 |
| 80, pH 7.0 | |
| Dual Chamber Cartridge (DCC) in Pen Injector | Prefilled syringe with 29 G staked needle in an |
| Supplied with a 29 G thin wall X 5 mm needle | Autoinjector |
| | Autoinjector controls depth of injection |

### EXAMPLES

The following examples illustrate various non-limiting aspects of this invention. For the following examples, unless noted otherwise albiglutide (ALB) can be referred to as SEQ ID NO:1 and/or albiglutide.

### Example 1

Results for four studies (Study 1.0; Study 1.1; Study 1.2; and Study 1.3) are presented in Example 1 herein. Formulations presented in each study are designated by a formulation number. The same formulation number may be used in more than one Study but does not always represent the same formulation. Tables presenting the results for each Study include the Study number in the table title.

The stability of albiglutide (ALB) was evaluated at various temperatures during four studies encompassing sixty unique formulations. The effects of buffer, polyol, sugar, polysorbate 80, and sodium octanoate on albiglutide stability were examined. In addition, the pH and protein concentration were varied. A formulation containing citrate, arginine, trehalose, and polysorbate 80 was found to confer sufficient stability to albiglutide in aqueous solution to justify further development. The target specification of relevance is >96% monomer by SEC after 18 months of storage at 2-8° C. Other acceptable target specification of relevance include, but are not limited to, 100%, ≥99%, ≥98%, ≥97%, ≥96%, ≥95%, ≥94%, ≥93%, ≥92%, ≥91%, ≥90% monomer by SEC after 18 months. Additionally, other acceptable target specification include the same monomer percentages by SEC after 12 months.

### Materials and Methods

### Materials

The chemicals used in this study are summarized in Table 1. The materials and equipment employed in this project are summarized in Table 2 and 3, respectively.

TABLE 1
Chemicals used in Study 1

| Chemical | Supplier | Purity |
|---|---|---|
| sodium citrate dihydrate | Mallinckrodt | USP |
| arginine hydrochloride | Spectrum | USP |
| trehalose dihydrate | Spectrum | unknown |
| polysorbate 80 | Spectrum | NF |
| sodium octanoate | Alfa Aesar | 96% |
| sodium phosphate monobasic | Fisher | ACS |
| monohydrate | | |
| trehalose dihydrate | Spectrum | unknown |
| mannitol | BDH | USP |
| histidine | Spectrum | USP |
| sodium succinate | Spectrum | unknown |

TABLE 2
Materials used in Study 1

| Material | Supplier | Material | Supplier |
|---|---|---|---|
| Millex 0.22 micron filter | Millipore | Phosphoric acid (85%) | Mallinckrodt Baker |
| unit | | Sodium Hydroxide | Mallinckrodt Baker |
| Vial 1 mL Fiolax Clear | Schott | Acetic Acid, Glacial | Mallinckrodt Baker |
| 13 mm aluminum seal | Wheaton | L-Arginine (≥98%) | Sigma Aldrich |
| 13 mm butyl stopper | Kimble-Chase | Iminodiacetic acid | Sigma Aldrich |
| HPLC Autosampler | National Scientific | Acquity UPLC BEH 300 | Waters |
| 12 × 32 mm vials | | C4 1.7 um 2.1 × 150 mm | |
| HPLC Vial Black Screw | Fisherbrand | Pellicon XL Biomax 10 | Millipore |
| Cap | | membrane filter | |
| HPLC Vial Polyspring | National Scientific | cIEF kit | Beckman |
| Inserts | | pI 5.5 cIEF marker | ProteinSimple |
| TSKgel G3000SWxl | Tosoh | pI 6.6 cIEF marker | American Peptide |
| cIEF Gel Polymer | Beckman Coulter | | |
| Solution | | | |
| Pharmalyte 3-10 | GE Healthcare | | |
| ampholyte | | | |
| Urea | Mallinckrodt Baker | | |

TABLE 3
Instruments and equipment used in Study

| Manufacturer | Instrument | Model |
|---|---|---|
| Denver Instrument | pH meter | Model 250 |
| Cary | UV-VIS spectrometer | Bio 100 |
| Dionex | HPLC | Ultimate 3000 |
| Dionex | HPLC | Ultimate 3000 |
| Dionex | HPLC | Ultimate 3000 |
| Eppendorf | centrifuge | MiniSpin Plus |
| BioRad | power supply | Power Pac 300 |
| Binder | Oven | Binder |
| Binder | Oven | Binder |
| Sartorius | balance | CPA124S |
| VWR | heating block | 12621-104 |
| Labnet | shaker | Orbit P4 |
| HP | CE | 3D-CE |

### Methods

The following sections describe the methods and techniques used in conducting these studies.

Sample Preparation.

Formulation buffers at the appropriate pH were prepared as two 1 L stock volumes for dialysis. For each formulation, a stock volume equal to 130% of the mass required was placed into a Slide-A-Lyzer cassette (10 kDa cutoff). Samples were dialyzed twice overnight at 2-8° C. in buffer (2×1 L). Next, samples were removed and concentrations determined by A₂₈₀. Samples were diluted to the appropriate concentration with dialysis buffer, and any other excipients (polysorbates, disaccharides, amino acids, octanoate, etc.) were added as solids to solution. Final concentrations were then measured by A₂₈₀. Samples were sterile filtered in a biosafety cabinet and placed into labeled, autoclaved vials and stoppered. Vials were then placed in stability chambers at the indicated temperature.

UV Spectroscopy.

All UV spectroscopy for this example, unless otherwise stated, was performed using a Varian Cary 100 Bio spectrometer. A calibrated 0.1 mm cell (0.0096 cm) was used for all readings. The instrument was zeroed against buffer before analysis at 280 nm. The demountable cell was rinsed with buffer, water, and ethanol then dried before each sample was analyzed. An extinction coefficient of 0.755 mL/mg cm was determined using the assigned concentration for albiglutide.

Size Exclusion Chromatography (SEC) Method.

Briefly, a Tosoh TSK Gel G3000SWxl 7.8 mm×30 cm column was used for all separations. The mobile phase consisted of 15 mM sodium phosphate, pH 7.5. A flow rate of 1 mL/min was used and the detection wavelength set to 214 nm. Samples were diluted to 1.7 mg/mL in water, and 54 (8.5 μg) of solution was injected for each separation. Each run was 20 minutes, and all sample components were separated in this amount of time. Following analysis, the column was rinsed with deionized water and stored in 20% methanol.

Reversed Phase Chromatography (RP HPLC) Method.

All separations were performed using an Acquity UPLC BEH300C4 1.7 μm 2.1×150 mm column. The column was held at 25° C. and 2 μg of protein was injected on column for each run from a sample diluted to 1 mg/mL (20 μL injection). A constant flow rate of 0.1 mL/min was employed. Solvent A consisted of 0.15% TFA in deionized water, and Solvent B consisted of 0.15% TFA in acetonitrile. Detection was performed at 214 nm. Buffer chromatograms were subtracted from sample chromatograms before integration.

The gradient schedule is as follows:

| Time | % B |
|---|---|
| 0 | 37.4 |
| 20 | 40.4 |
| 20.1 | 95 |
| 25 | 95 |
| 25.1 | 37.4 |
| 60 | 37.4 |

Capillary Isoelectric Focusing (cIEF) Method.

Capillary isoelectric focusing was conducted using insight from the PA 800 plus Application Guide published by Beckman Coulter and the document “cIEF.PDF” which is internal guidance. Samples were prepared to contain the following:
100 μL of 10M urea
50 mM DTT
6 μL Pharmalyte pH 5-8
6 uL 500 mM arginine
0.5 μL pI 5.12 marker (ProteinSimple)
0.5 μL pI 6.60 marker (American Peptide, 5 mg/mL)
0.5 μL 100 mg/mL albiglutide sample (or equivalent mass)

Neutral coated capillaries were prepared as described by Gao, et al. (Gao, L.; Liu, S. R., Cross-linked polyacrylamide coating for capillary isoelectric focusing. Anal Chem 2004, 76 (24), 7179-7186.). Coated capillaries were cut to a length of 32 cm, and a window created for detection at 22 cm. Capilliary was rinsed with 4 M urea (3 min) and dionized water (2 min) before each run. Whole capillary injection of the sample was performed (100 seconds at 25 psi). The sample was focused for 15 minutes between anolyte at the inlet (100 mM phosphoric acid in 0.5% methylcellulose) and catholyte at the outlet (100 mM sodium hydroxide in 0.5% methylcellulose) at a constant voltage of 25 kV, normal polarity. Chemical mobilization was performed between the anolyte and a mobilizer solution (350 mM acetic acid) for 40 minutes at a constant voltage of 30 kV. Detection was performed at 280 nm. Each run was ended with a 2 minute flush with deionized water. cIEF was omitted from Study 1.3.

Data was analyzed using Chromeleon software. Relative area and first peak moment were recorded for all integrated peaks in the sample. Isoelectric points were determined using the first peak moments of the two cIEF markers.

SDS-Page Method.

Different versions of the method were employed at various stages of Study 1, as described below.
Study 1.0, 1.1: Samples were prepared for SDS-PAGE using 0.5 μL of sample, 2.5 μL NuPAGE LDS Sample Buffer, and 7 μL water. Each prepared sample was incubated at 70° C. for 10 minutes prior to electrophoresis. NuPAGE 12% Bis-Tris gels and NuPAGE MES buffer were used for each gel. Samples were loaded into each well (10 μL) and separated for 40 minutes at 200 V. Gels were then rinsed with deionized water and soaked in deionized water for 15 minutes. Gels were then covered with Simply Blue Safe Stain (Invitrogen) for one hour, and then incubated in deionized water again for at least 2 hours.
Study 1.2: As above, but each 10 μL sample was prepared with 0.25 μg protein regardless of sample protein concentration. Only 8 μL of material is loaded into each gel well.
Study 1.3: SDS-PAGE was not performed for this study.

Projection to Latent Structures (PLS) Method.

Detailed descriptions of PLS modeling have been published [Stahle, L.; Wold, K. Multivariate data analysis and experimental design in biomedical research. Prog. Med. Chem. 1988, 25: 291-338; Wold S. PLS-regression: a basic tool of chemometrics. Chemom. Intell. Lab. Syst. 2001, 58: 109-130]. It is the most common chemometric method for multivariate analysis of large and complex data sets. For any large matrix of values, where there are a reasonable number of samples (together forming the so-called X-matrix), mathematical models can be constructed that explain the largest amount of variance in the dependent variable(s) of interest (the Y-matrix). The best single description of the relationship between the variation in the X-matrix and the endpoint (the Y matrix) is called the first principal component, PC1. The next important (in terms of describing the variance in the Y-matrix) component is called the second principal component, PC2, and so on. Quite often, only one or two PCs are required to explain most of the variance in the Y-matrix. Each of these PCs contains some contribution from each of the variables in the X-matrix. If a variable within the X-matrix contributes heavily to the construction of a given PC, then it is ranked as being significant. In fact, regression coefficients can be calculated for each variable in the X-matrix for a given model, where a model is the composite of a certain number of PCs in order to provide an adequate description of the Y-matrix [Katz, M. H. Multivariate Analysis: A Practice Guide for Clinicians. Cambridge University Press, New York, pp. 158-162 (1999)]. In summary, PLS takes information from the X-matrix, calculates the desired number of PCs, and constructs a suitable model. A PLS model can include interaction terms, when appropriate, as well as quadratic terms, which allows one to characterize non-linear effects. The model that includes all of the samples is termed a calibration model [Wold S. PLS-regression: a basic tool of chemometrics. Chemom. Intell. Lab. Syst. 2001, 58: 109-130]. The overall coefficient of determination (r²) indicates the quality of the model. All PLS calculations were conducted using Unscrambler® software (CAMO, Corvallis, Oreg.). A PLS analysis done with a single variable in the Y-matrix is termed PLS1 analysis. Building a model that fits multiple variables in the Y-matrix is called PLS2 analysis.

A full cross validation was performed on all calibration models using standard techniques [Martens, H.; Martens, M. Multivariate Analysis of Quality: An Introduction, Wiley and Sons, Chichester, UK (2001)]. Briefly, one sample is removed at a time, the data set is recalibrated, and a new model is constructed. This process is repeated until all of the calibration samples are removed once and quantified as a validation model. Therefore, the first set, containing all samples is referred to as the calibration set and the one after cross-validation as the validation set. The jack-knife algorithm [Martens, H.; Martens, M. Multivariate Analysis of Quality: An Introduction, Wiley and Sons, Chichester, UK (2001)] was used to determine statistical significance for any factor used in constructing the PLS models described above.

### Results and Discussion

The project proceeded through four rounds of formulation screening. Results from each round were used to optimize formulations in the next round.

### Study 1.0.

The first round of screening was designed to investigate both buffer species and pH. Using 12 formulations, three different buffers (citrate, phosphate, histidine) were examined between pH values of 5.7-6.8. In addition, two formulations contained 10 mM sodium octanoate, identified as a potential stabilizer in preformulation work done at GSK. One of the formulations in this study contained mannitol, trehalose, arginine, and polysorbate 80. Samples in glass vials were stored for one and two weeks at 40° C. for this study. All samples contained albiglutide at a concentration of 60 mg/mL.

TABLE 4
Composition of Study 1.0 formulations

| Form | | citrate | phosphate | His | mann | tre | PS80 | octanoate |
|---|---|---|---|---|---|---|---|---|
| No | pH | mM | mM | mM | mM | mM | % | mM |
| F01 | 7.0 | 0 | 10 | 0 | 153 | 117 | 0.01 | 0 |
| F02 | 5.7 | 10 | 0 | 0 | 0 | 0 | 0 | 0 |
| F03 | 5.7 | 0 | 10 | 0 | 0 | 0 | 0 | 0 |
| F04 | 5.7 | 0 | 0 | 10 | 0 | 0 | 0 | 0 |
| F05 | 6.3 | 0 | 10 | 0 | 0 | 0 | 0 | 0 |
| F06 | 6.3 | 0 | 0 | 10 | 0 | 0 | 0 | 0 |
| F07 | 5.3 | 10 | 0 | 0 | 0 | 0 | 0 | 0 |
| F08 | 5.3 | 0 | 0 | 10 | 0 | 0 | 0 | 0 |
| F09 | 5.7 | 0 | 10 | 0 | 0 | 0 | 0 | 10 |
| F10 | 5.7 | 10 | 0 | 0 | 0 | 0 | 0 | 10 |
| F11 | 6.8 | 0 | 10 | 0 | 0 | 0 | 0 | 0 |
| F12 | 6.8 | 0 | 0 | 10 | 0 | 0 | 0 | 0 |

TABLE 5
Study 1.0 Visual Observations
LB141 S1 Visual Observations

| Sample | t0 | 1 wk 40 C. | 2 wk 40 C. |
|---|---|---|---|
| F01 | clear | clear | clear |
| F02 | clear | clear | clear |
| F03 | clear | cloudy | cloudy |
| F04 | clear | cloudy | gelled |
| F05 | clear | clear | cloudy |
| F06 | clear | cloudy | gelled |
| F07 | clear | clear | clear |
| F08 | clear | gelled | gelled |
| F09 | clear | clear | clear |
| F10 | clear | clear | clear |
| F11 | clear | clear | clear |
| F12 | clear | cloudy | cloudy |

Initially, all samples were clear, with a light brown color at the time of preparation. At the indicated incubation times, several samples had become either cloudy or had completely gelled (Table 5). Samples that displayed poor visual appearance were not analyzed further.

TABLE 6
Purity by RP HPLC for formulations from Study 1.0
LB141 Study 1 RP

| Sample | t0 | | | t1 (1 wk 40° C.) | | | t2 (2wk 40° C.) | | |
|---|---|---|---|---|---|---|---|---|---|
| F01 | 85.18 | ± | 0.82 | 77.46 | ± | 1.46 | 71.29 | ± | 0.56 |
| F02 | 87.07 | ± | 0.71 | 83.86 | ± | 0.77 | 81.08 | ± | 0.14 |
| F03 | 85.01 | ± | 1.18 | n/a | ± | n/a | n/a | ± | n/a |
| F04 | 85.97 | ± | 1.02 | n/a | ± | n/a | n/a | ± | n/a |
| F05 | 85.12 | ± | 0.40 | 74.19 | ± | 0.87 | n/a | ± | n/a |
| F06 | 85.98 | ± | 0.33 | n/a | ± | n/a | n/a | ± | n/a |
| F07 | 89.37 | ± | 0.19 | 85.51 | ± | 0.35 | 79.93 | ± | 0.75 |
| F08 | 85.84 | ± | 0.22 | n/a | ± | n/a | n/a | ± | n/a |
| F09 | 88.55 | ± | 0.97 | 86.67 | ± | 0.31 | 82.76 | ± | 0.48 |
| F10 | 88.13 | ± | 0.96 | 85.23 | ± | 0.30 | 81.32 | ± | 0.31 |
| F11 | 87.24 | ± | 0.81 | 75.21 | ± | 0.53 | 68.79 | ± | 0.35 |
| F12 | 84.67 | ± | 0.81 | n/a | ± | n/a | n/a | ± | n/a |

TABLE 7
Monomer content by SEC for formulations from Study 1.0
LB141 Study 1 SEC

| Sample | t0 | | | t1 (1 wk 40° C.) | | | t2 (2 wk 40° C.) | | |
|---|---|---|---|---|---|---|---|---|---|
| F01 | 99.05 | ± | 0.09 | 90.12 | ± | 0.17 | 83.95 | ± | 0.28 |
| F02 | 98.70 | ± | 0.00 | 96.18 | ± | 0.02 | 93.65 | ± | 0.23 |
| F03 | 98.93 | ± | 0.04 | 96.18 | ± | 0.01 | n/a | ± | n/a |
| F04 | 98.42 | ± | 0.16 | 50.08 | ± | 0.38 | n/a | ± | n/a |
| F05 | 98.91 | ± | 0.02 | 84.79 | ± | 0.31 | n/a | ± | n/a |
| F06 | 98.85 | ± | 0.04 | 83.21 | ± | 0.11 | n/a | ± | n/a |
| F07 | 98.51 | ± | 0.04 | 91.29 | ± | 0.05 | 85.86 | ± | 0.12 |
| F08 | 98.67 | ± | 0.13 | n/a | ± | n/a | n/a | ± | n/a |
| F09 | 98.83 | ± | 0.01 | 97.24 | ± | 0.02 | 96.40 | ± | 0.04 |
| F10 | 98.54 | ± | 0.00 | 97.36 | ± | 0.03 | 96.62 | ± | 0.08 |
| F11 | 98.91 | ± | 0.03 | 90.33 | ± | 0.33 | 82.17 | ± | 0.16 |
| F12 | 98.80 | ± | 0.03 | 56.64 | ± | 0.65 | n/a | ± | n/a |

The purity of the Study 1.0 samples by RP HPLC shows two formulations (F09 and F10) faring better than the others (Table 6), as shown in the table. These are the two containing 10 mM octanoate. It also appears that raising the pH to ˜6.8 reduced the stability. A similar trend was seen by SEC, where the monomer content upon storage was highest for these two formulations (Table 7). These formulations, outside of the control formulation (see lyophilized formulation and F01 in Table 4), F01, only contain buffer and no other stabilizer beside octanoate in Formulations F09 and F10. In terms of formulations that did not show poor visual appearance, most were based on citrate.

TABLE 8
Purity by RP HPLC for samples from Study 1.0
subjected to Freeze-Thaw cycling
LB141 Freeze/Thaw RP

| Sample | 3x Freeze | | | 5x Freeze | | |
|---|---|---|---|---|---|---|
| F01 | 84.40 | ± | 1.84 | 84.37 | ± | 0.77 |
| F02 | 87.56 | ± | 1.97 | 88.21 | ± | 0.17 |
| F03 | 84.07 | ± | 1.16 | 85.35 | ± | 1.06 |
| F04 | 83.13 | ± | 2.15 | 84.69 | ± | 0.65 |
| F05 | 86.29 | ± | 1.16 | 85.53 | ± | 1.04 |
| F06 | 85.59 | ± | 0.93 | 87.78 | ± | 0.60 |
| F07 | 87.11 | ± | 0.79 | 89.89 | ± | 0.68 |
| F08 | 85.00 | ± | 0.99 | 87.56 | ± | 0.60 |
| F09 | 88.41 | ± | 2.18 | 89.32 | ± | 0.43 |
| F10 | 88.10 | ± | 0.36 | 89.34 | ± | 0.59 |
| Fl 1 | 85.31 | ± | 0.71 | 86.49 | ± | 0.37 |
| F12 | 88.01 | ± | 0.70 | 87.83 | ± | 0.53 |

These formulations were also subjected to repeated freeze-thaw (F/T) cycles. Again, the stability was determined using RP HPLC (Table 8) and SEC (Table 9). Within the assay error, there were no losses in purity upon freeze-thaw cycling. There were slight apparent decreases in monomer content by SEC (Table 9), but these were small enough that they might not be significant.

TABLE 9
Monomer content by SEC for samples from Study
1.0 subjected to freeze-thaw cycling
LB141 Freeze/Thaw SEC

| Sample | 3x Freeze | | | 5x Freeze | | |
|---|---|---|---|---|---|---|
| F01 | 99.03 | ± | 0.01 | 99.00 | ± | 0.03 |
| F02 | 98.62 | ± | 0.01 | 98.64 | ± | 0.00 |
| F03 | 98.71 | ± | 0.05 | 98.60 | ± | 0.05 |
| F04 | 97.94 | ± | 0.17 | 97.30 | ± | 0.21 |
| F05 | 98.83 | ± | 0.01 | 98.79 | ± | 0.02 |
| F06 | 98.56 | ± | 0.06 | 98.58 | ± | 0.05 |
| F07 | 98.39 | ± | 0.04 | 98.30 | ± | 0.01 |
| F08 | 98.11 | ± | 0.21 | 97.57 | ± | 0.18 |
| F09 | 98.75 | ± | 0.02 | 98.70 | ± | 0.01 |
| F10 | 98.54 | ± | 0.01 | 98.57 | ± | 0.01 |
| F11 | 98.81 | ± | 0.01 | 98.72 | ± | 0.02 |
| F12 | 98.71 | ± | 0.02 | 98.34 | ± | 0.05 |

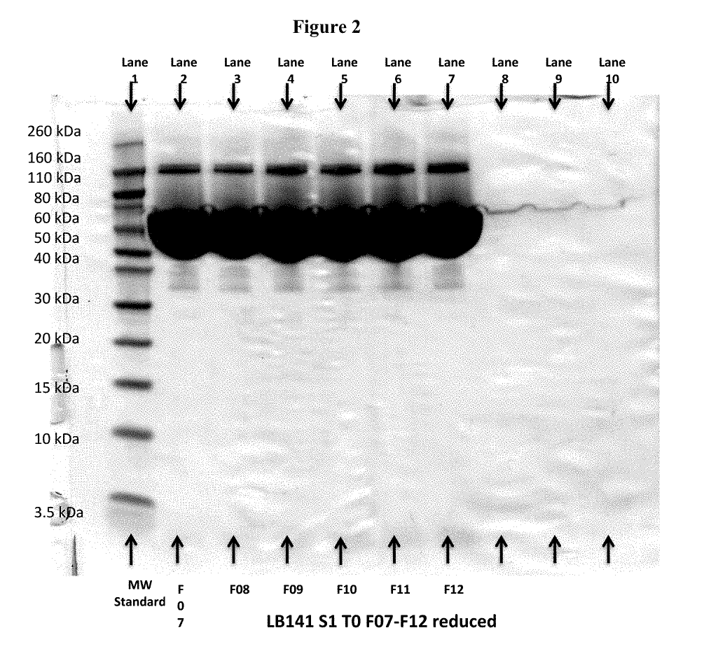

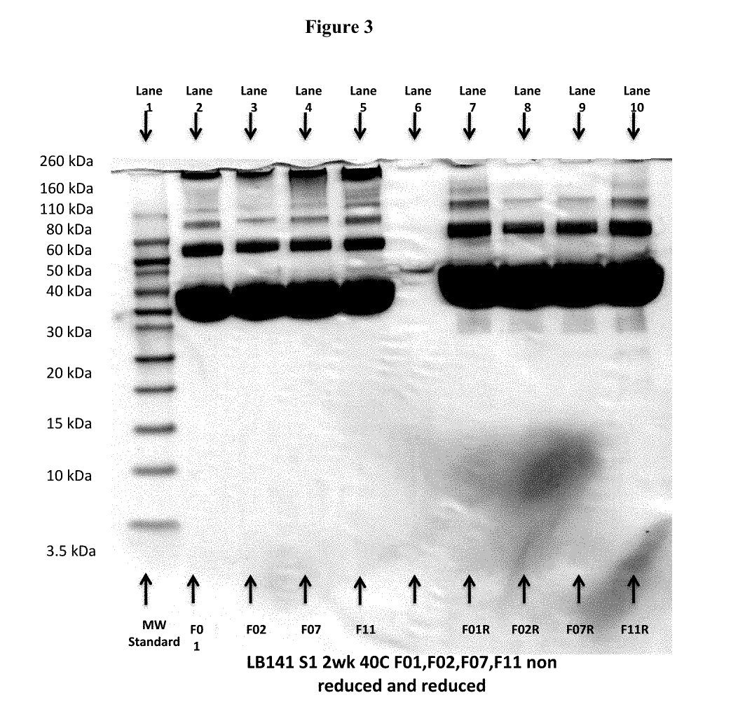

Finally, Study 1.0 samples were evaluated using SDS-PAGE. This assay can be run under reducing or non-reducing conditions. The gels were intentionally overloaded in order to monitor the higher molecular weight (MW) bands (FIGS. 1 and 2). The reduced gels (FIG. 2) show loss of the highest MW band seen in the non-reduced gels (FIG. 1), suggesting this is due to some type of disulfide cross-linking that is present even at t0. After two weeks at 40° C., there is a very high MW species that barely enters the non-reduced gel (FIG. 3). This species is effectively removed by adding a reducing agent. In addition, there does appear to be some differences in the amount of aggregate seen in the non-reduced gels, so only these were used for subsequent studies.

### Study 1.1.

The second round of study focused on a somewhat different pH range and specific buffer compositions, mostly without other stabilizers present (Table 10). The pH range was lowered, covering between 5.3 and 6.0, as the previous work suggested that a lower pH would be beneficial for stability. Histidine, which led to poor solution stability (gelation, haziness) in Study 1.0, was removed from further consideration. In this pH range, carboxylate buffers, like citrate, should work well. Because citrate appeared to confer some stability, succinate was also introduced as a buffer to evaluate due to its structural similarity. Trehalose, mannitol, and sodium octanoate were each examined as stabilizers. As in Study 1.0, the first formulation is the lyophilized control formulation (see F01 of Table 4). In addition, formulations F13 and F14 include mannitol and trehalose, respectively, to determine what their individual contributions might be. Samples were evaluated after a week at 40° C. and two weeks at 25° C.

TABLE 10
Composition of Study 1.1 formulations

| Form No | pH | citrate | phosphate | succinate | mannitol | trehalose | PS80 | octanoate |
|---|---|---|---|---|---|---|---|---|
| F01 | 7.0 | 0 | 10 | 0 | 153 | 117 | 0.01 | 0 |
| F02 | 5.7 | 10 | 0 | 0 | 0 | 0 | 0.01 | 0 |
| F03 | 5.7 | 20 | 0 | 0 | 0 | 0 | 0.01 | 0 |
| F04 | 5.7 | 10 | 0 | 0 | 0 | 0 | 0.01 | 10 |
| F05 | 6.0 | 10 | 0 | 0 | 0 | 0 | 0.01 | 10 |
| F06 | 5.3 | 10 | 0 | 0 | 0 | 0 | 0.01 | 0 |
| F07 | 5.3 | 0 | 0 | 10 | 0 | 0 | 0.01 | 0 |
| F08 | 5.7 | 0 | 10 | 0 | 0 | 0 | 0.01 | 0 |
| F09 | 5.7 | 0 | 0 | 10 | 0 | 0 | 0.01 | 0 |
| F10 | 5.7 | 0 | 0 | 20 | 0 | 0 | 0.01 | 10 |
| F11 | 6.0 | 0 | 0 | 10 | 0 | 0 | 0.01 | 10 |
| F12 | 6.0 | 0 | 0 | 10 | 0 | 0 | 0.01 | 0 |
| F13 | 5.7 | 10 | 0 | 0 | 153 | 0 | 0.01 | 0 |
| F14 | 5.7 | 10 | 0 | 0 | 0 | 117 | 0.01 | 0 |

TABLE 11
Purity by RP HPLC of samples from Study 1.1
LB141 S1.1 t0 RP

| Sample | t0 | | | content | 1 wk 40 C. | | | content | 2 wk 25 C. | | | content |
|---|---|---|---|---|---|---|---|---|---|---|---|---|
| F01 | 82.91 | ± | 0.22 | 100.00 | 80.08 | ± | 0.68 | 94.21 | 89.39 | ± | 0.09 | 19.15 |
| F02 | 87.86 | ± | 1.25 | 100.00 | 81.44 | ± | 0.21 | 89.61 | 86.25 | ± | 0.22 | 78.31 |
| F03 | 87.37 | ± | 0.15 | 100.00 | 85.62 | ± | 0.69 | 100.07 | 84.40 | ± | 0.47 | 81.51 |
| F04 | 87.99 | ± | 0.91 | 100.00 | 83.02 | ± | 0.10 | 97.83 | 84.24 | ± | 0.87 | 81.84 |
| F05 | 86.13 | ± | 1.05 | 100.00 | 80.87 | ± | 0.05 | 100.98 | 83.12 | ± | 0.43 | 84.26 |
| F06 | 86.97 | ± | 0.56 | 100.00 | 83.06 | ± | 0.50 | 96.36 | 83.92 | ± | 0.43 | 72.53 |
| F07 | 87.07 | ± | 0.82 | 100.00 | 74.66 | ± | 0.87 | 102.50 | 16.77 | ± | 13.51 | 22.02 |
| F08 | 85.50 | ± | 0.23 | 100.00 | 72.23 | ± | 0.57 | 99.81 | 75.41 | ± | 1.23 | 32.03 |
| F09 | 85.41 | ± | 0.87 | 100.00 | 77.39 | ± | 0.88 | 100.19 | 63.73 | ± | 0.50 | 41.34 |
| F10 | 86.46 | ± | 0.92 | 100.00 | 81.73 | ± | 0.66 | 99.16 | 83.09 | ± | 1.47 | 81.94 |
| F11 | 85.76 | ± | 0.39 | 100.00 | 81.32 | ± | 0.14 | 99.96 | n/a | ± | n/a | n/a |
| F12 | 83.50 | ± | 0.46 | 100.00 | 76.81 | ± | 0.31 | 101.96 | 57.49 | ± | 0.91 | 33.07 |
| F13 | 86.14 | ± | 0.16 | 100.00 | 83.76 | ± | 0.46 | 96.87 | 82.50 | ± | 0.44 | 70.89 |
| F14 | 87.32 | ± | 1.23 | 100.00 | 83.62 | ± | 0.82 | 97.57 | 65.00 | ± | 1.56 | 68.85 |

TABLE 12
Monomer content by SEC of samples from Study 1.1
LB141 S2 t0 SEC

| Sample | t0 | | | 1 wk 40 C. | | | 2 wk 25 C. | | |
|---|---|---|---|---|---|---|---|---|---|
| F01 | 98.46 | ± | 0.06 | 89.10 | ± | 0.44 | 94.60 | ± | 0.34 |
| F02 | 98.36 | ± | 0.01 | 94.46 | ± | 0.03 | 97.54 | ± | 0.02 |
| F03 | 98.62 | ± | 0.01 | 96.29 | ± | 0.02 | 97.76 | ± | 0.01 |
| F04 | 98.54 | ± | 0.02 | 97.40 | ± | 0.01 | 98.01 | ± | 0.01 |
| F05 | 98.60 | ± | 0.02 | 97.59 | ± | 0.01 | 98.06 | ± | 0.02 |
| F06 | 98.31 | ± | 0.01 | 89.66 | ± | 0.03 | 97.26 | ± | 0.01 |
| F07 | 98.10 | ± | 0.02 | 78.76 | ± | 0.79 | 97.80 | ± | 0.02 |
| F08 | 98.57 | ± | 0.01 | 76.86 | ± | 0.05 | 97.64 | ± | 0.00 |
| F09 | 98.40 | ± | 0.00 | 90.06 | ± | 0.01 | 97.62 | ± | 0.03 |
| F10 | 98.57 | ± | 0.01 | 96.71 | ± | 0.01 | 97.77 | ± | 0.04 |
| F11 | 98.73 | ± | 0.04 | 97.40 | ± | 0.01 | 93.62 | ± | 0.11 |
| F12 | 98.75 | ± | 0.04 | 89.17 | ± | 0.04 | 97.53 | ± | 0.04 |
| F13 | 98.73 | ± | 0.00 | 96.06 | ± | 0.02 | 97.89 | ± | 0.03 |
| F14 | 98.75 | ± | 0.02 | 95.81 | ± | 0.03 | 97.65 | ± | 0.01 |

The purity of albiglutide by RP is initially near 85-87% for these formulations, with the control formulation being somewhat lower at t0 (Table 11). As samples are stored at elevated temperatures, the purity by RP HPLC decreases, with the most stable formulations containing citrate (Table 11). The monomer content values also decrease upon storage, with the low pH samples (pH 5.3), formulations F07 and F08, showing the biggest decreases after one week at 40° C. (t1) (Table 12). After two weeks at 25° C. (t2), the monomer contents were all at 97% or greater, except for formulations F01 and F11. The result for F11 seems to be out of line with the other values, and may not be correct. Some formulations did show some haziness upon storage and these are noted in bold in Tables 11 and 12.

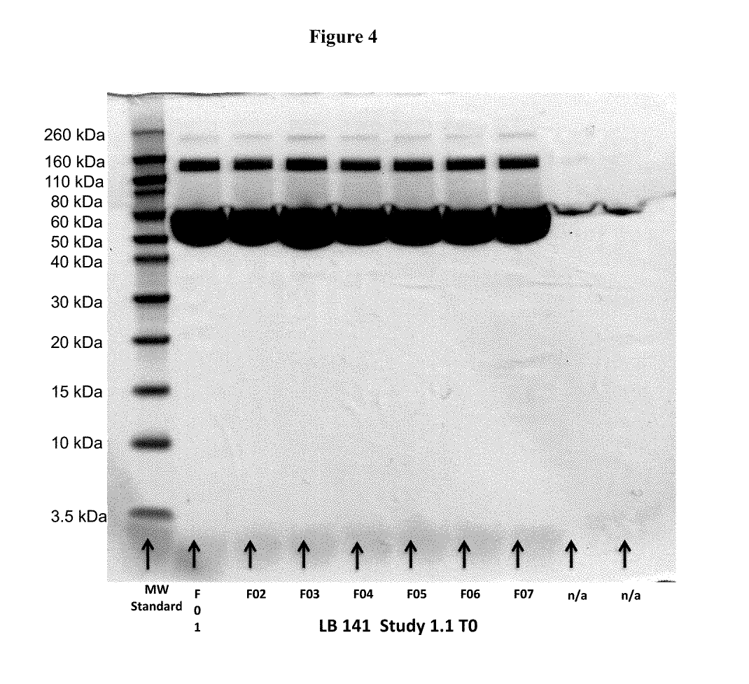

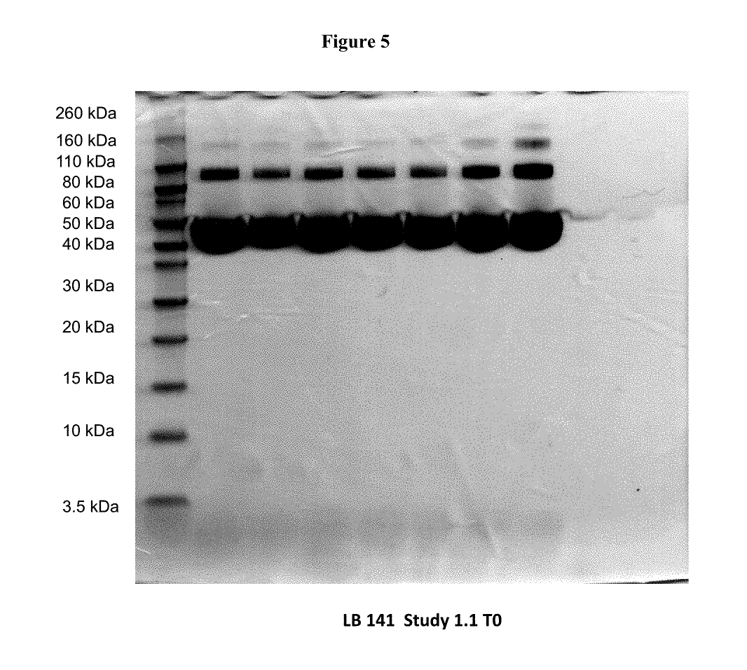

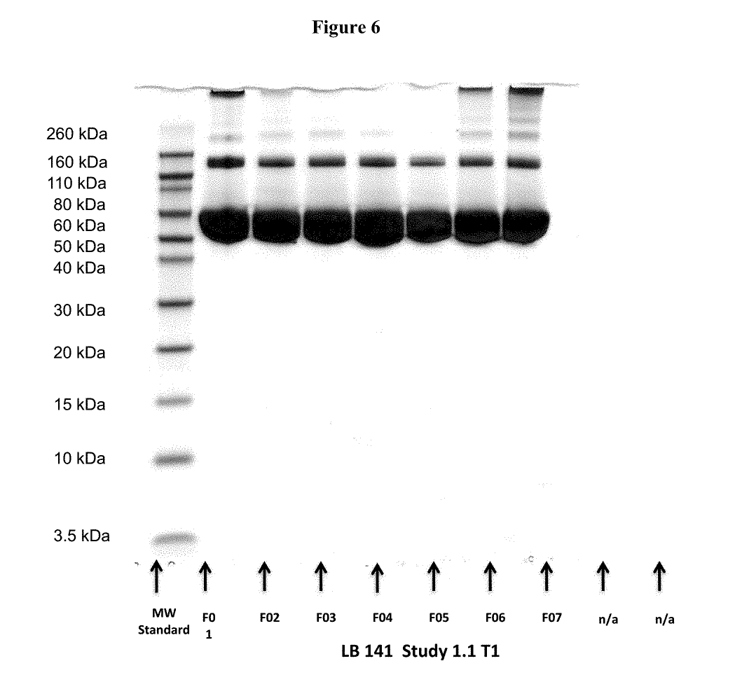

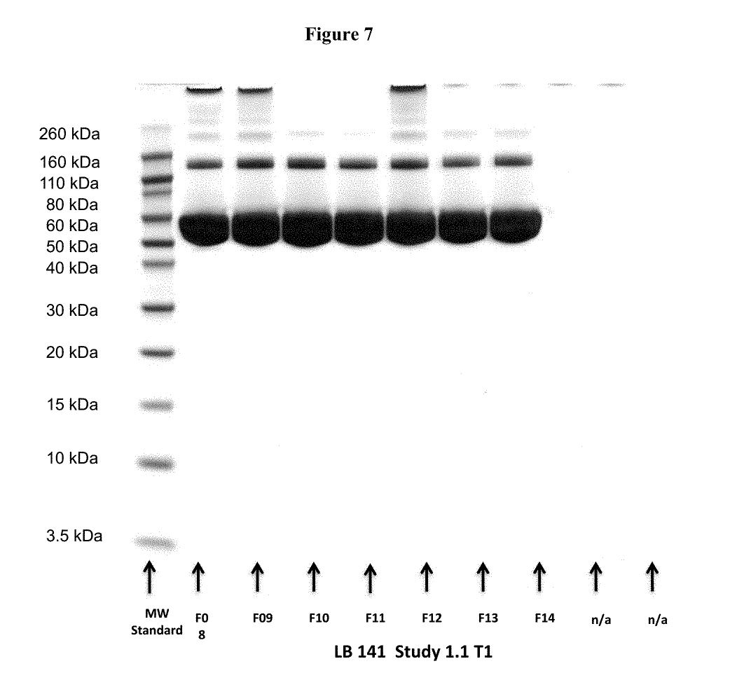

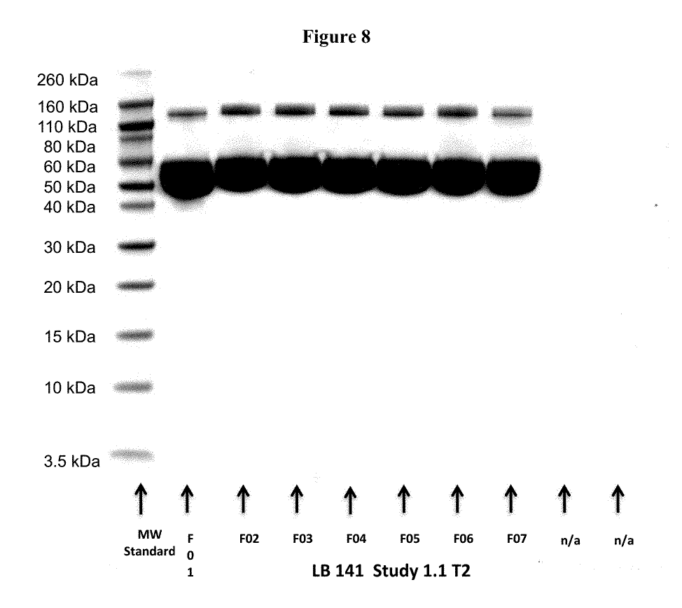

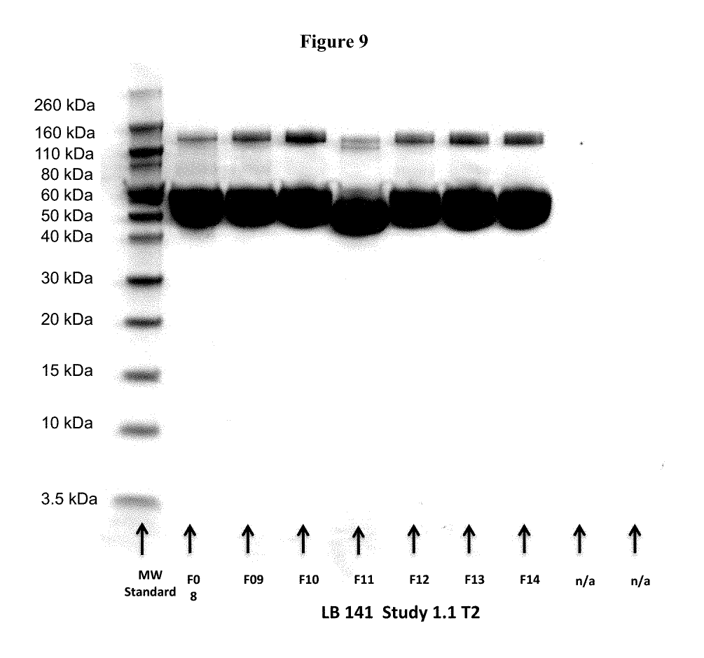

These formulations were also assayed using non-reducing SDS-PAGE gels. The t0 data gels are shown in FIGS. 4 and 5. There is strong band above the monomer that runs as if it were a dimer and a faint band of higher MW that is present in all of the formulations. This band is quite pronounced in formulation F14 (FIG. 5). At t1, there is much greater increase in high MW species in formulations F06 and F07 than any of the other formulations from F01 to F07 (FIG. 6). These are the low pH formulations. Similarly, there is an increased amount of high MW species in formulations F08, F09, and F12. The latter two preparations contain succinate buffer. In addition, the lowest amount of these species appears to be in the octanoate formulations (FIGS. 6 and 7). At t2 (two weeks, 25° C., there is very little degradation seen by SDS-PAGE (FIGS. 8 and 9), commensurate with the small amount of degradation seen by RP HPLC and SEC under these conditions.

Both the SDS-PAGE and SEC indicate that aggregation, not fragmentation, is the primary route of degradation. Overall, Study 1.1 demonstrates that citrate is likely the best buffer, with succinate as a possible alternative. Although the pH response appears to be relatively flat, the optimum is likely to be near pH 5.7.

### Study 1.2.

The third study in this round was designed mainly to optimize levels of formulation components found to be stabilizers in the first two rounds. Specifically, this study examined levels of citrate, succinate, octanoate, trehalose, and mannitol. In addition, protein concentrations up to 100 mg/mL were evaluated. Note that each formulation now contains 100 mM arginine HCl, designed to match what is being used in the additional studies on albiglutide, which have been termed Study 2. Finally, two formulations were made from polysorbate-80 (PS-80)-free material (all previous stock solutions contained 0.01% PS-80 added during processing). Samples were examined after incubations at 40° C. for one week (t1), 25° C. for two weeks (t2), and 25° C. for four weeks (t4).

TABLE 13
Composition of Study 1.2 formulations

| | | arginine | protein | citrate | succinate | octanoate | mannitol | trehalose | PS 80 |
|---|---|---|---|---|---|---|---|---|---|
| Formulation | pH | mM | mg/mL | mM | mM | mM | mM | Mm | wt % |
| F01 | 5.5 | 100 | 60 | 10 | 0 | 10 | 240 | 0 | 0.01 |
| F02 | 5.5 | 100 | 60 | 0 | 10 | 10 | 0 | 240 | 0.01 |
| F03 | 5.5 | 100 | 100 | 0 | 10 | 10 | 240 | 0 | 0.01 |
| F04 | 5.5 | 100 | 100 | 10 | 0 | 20 | 0 | 240 | 0.01 |
| F05 | 5.5 | 100 | 60 | 5 | 0 | 0 | 120 | 0 | 0.01 |
| F06 | 5.5 | 100 | 60 | 15 | 0 | 0 | 120 | 0 | 0.01 |
| F07 | 5.9 | 100 | 60 | 10 | 0 | 10 | 240 | 0 | 0.01 |
| F08 | 5.9 | 100 | 60 | 0 | 10 | 10 | 0 | 240 | 0.01 |
| F09 | 5.9 | 100 | 60 | 0 | 10 | 0 | 240 | 0 | 0.01 |
| F10 | 5.9 | 100 | 60 | 10 | 0 | 10 | 0 | 240 | 0.01 |
| F11 | 5.9 | 100 | 100 | 5 | 0 | 0 | 240 | 0 | 0.01 |
| F12 | 5.9 | 100 | 100 | 20 | 0 | 20 | 240 | 0 | 0.01 |
| F13 | 5.7 | 100 | 60 | 10 | 0 | 10 | 120 | 0 | 0 |
| F14 | 5.7 | 100 | 60 | 10 | 0 | 0 | 120 | 0 | 0 |
| F15 | 5.7 | 100 | 60 | 0 | 20 | 0 | 0 | 120 | 0.01 |
| F16 | 5.7 | 100 | 100 | 0 | 20 | 0 | 0 | 120 | 0.01 |

TABLE 14
pH values and protein concentration by UV for
formulations from Study 1.2
LB141 S1.2 pH and UV

| | | | t1 1 wk 40 C. | | t2 2 wk 25 C. | | t4 4 wk 25 C. | |
|---|---|---|---|---|---|---|---|---|
| | t0 | | | conc | | conc | | conc |
| Sample | pH | conc (mg/mL) | pH | (mg/mL) | pH | (mg/mL) | pH | (mg/mL) |
| F01 | 5.56 | 60.42 | 5.41 | 61.82 | 5.55 | 60.13 | 5.46 | 58.84 |
| F02 | 5.57 | 63.31 | 5.60 | 62.87 | 5.62 | 61.52 | 5.55 | 58.06 |
| F03 | 5.46 | 100.00 | 5.47 | 103.30 | 5.57 | 102.59 | 5.46 | 101.90 |
| F04 | 5.54 | 79.30 | 5.55 | 83.25 | 5.66 | 80.71 | 5.60 | 80.53 |
| F05 | 5.44 | 60.83 | 5.44 | 58.95 | 5.57 | 59.84 | 5.47 | 59.64 |
| F06 | 5.41 | 58.65 | 5.42 | 59.71 | 5.52 | 59.42 | 5.46 | 59.75 |
| F07 | 5.84 | 60.67 | 5.87 | 62.44 | 5.95 | 62.50 | 5.79 | 60.65 |
| F08 | 6.03 | 60.90 | 6.05 | 62.72 | 6.11 | 62.27 | 6.00 | 61.35 |
| F09 | 5.87 | 61.31 | 6.01 | 63.76 | 5.95 | 62.67 | 5.94 | 61.69 |
| F10 | 5.89 | 61.82 | 5.98 | 63.67 | 5.99 | 63.80 | 5.94 | 60.69 |
| F11 | 5.80 | 102.25 | 5.73 | 102.50 | 5.93 | 103.31 | 5.75 | 101.17 |
| F12 | 5.93 | 97.89 | 5.92 | 99.99 | 6.00 | 99.71 | 6.01 | 98.61 |
| F13 | 5.78 | 57.23 | 5.73 | 57.13 | 5.92 | 57.99 | 5.78 | 56.91 |
| F14 | 5.63 | 56.42 | 5.57 | 58.76 | 5.72 | 56.88 | 5.65 | 57.17 |
| F15 | 5.71 | 58.35 | 5.57 | 58.64 | 5.72 | 58.94 | 5.62 | 58.31 |
| F16 | 5.61 | 102.69 | 5.56 | 99.81 | 5.71 | 101.50 | 5.65 | 98.84 |

There is little change in the pH or in the protein concentration for any of the formulations evaluated in Study 1.2 (Table 14). The initial monomer content by SEC for these formulations is between 98% and 99% (Table 15). The remainder is oligomer, possibly the dimer, which elutes at a relative retention time (RRT) of 0.89.

TABLE 15
Percentage of peak areas by SEC for formulations in Study 1.2 at t0
LB141 S1.2 t0 SEC

| | HMW1 | | | monomer | | |
|---|---|---|---|---|---|---|
| Sample | RRT 0.89 | | | RRT 1.00 | | |
| F01 | 1.56 | ± | 0.06 | 98.44 | ± | 0.06 |
| F02 | 1.66 | ± | 0.01 | 98.34 | ± | 0.01 |
| F03 | 1.66 | ± | 0.01 | 98.34 | ± | 0.01 |
| F04 | 1.55 | ± | 0.01 | 98.45 | ± | 0.01 |
| F05 | 1.35 | ± | 0.06 | 98.65 | ± | 0.06 |
| F06 | 1.53 | ± | 0.02 | 98.47 | ± | 0.02 |
| F07 | 1.53 | ± | 0.01 | 98.47 | ± | 0.01 |
| F08 | 1.55 | ± | 0.07 | 98.45 | ± | 0.07 |
| F09 | 1.52 | ± | 0.05 | 98.48 | ± | 0.05 |
| F10 | 1.45 | ± | 0.01 | 98.55 | ± | 0.01 |
| F11 | 1.49 | ± | 0.01 | 98.51 | ± | 0.01 |
| F12 | 1.69 | ± | 0.02 | 98.31 | ± | 0.02 |
| F13 | 1.13 | ± | 0.01 | 98.87 | ± | 0.01 |
| F14 | 1.16 | ± | 0.01 | 98.84 | ± | 0.01 |
| F15 | 1.76 | ± | 0.00 | 98.24 | ± | 0.00 |
| F16 | 1.72 | ± | 0.04 | 98.28 | ± | 0.04 |

Upon storage for one week at 40° C., the monomer content for most of the formulations decreases to about 97% or more (Table 16). Two new species are observed with a RRT 0.78 and 0.85, which are presumably higher molecular weight (MW) aggregates. A few formulations appear to retain more monomer than the others. These include F13, F14, F10, and F07. All contain citrate as the buffer and three of the four employ mannitol instead of trehalose as the polyol stabilizer. While the range of monomer contents is relatively small at t1 (Table 16), these differences are probably meaningful, especially when considering what formulations appear to contain no HMW3 species (RRT 0.78).

TABLE 16
Percentage of peak areas by SEC for formulations
in Study 1.2 at t1 (one week at 40° C.)
LB141 S1.2 t1 1 wk 40 C. SEC

| | HMW3 | | | HMW2 | | | HMW1 | | | monomer | | |
|---|---|---|---|---|---|---|---|---|---|---|---|---|
| Sample | RRT 0.78 | | | RRT 0.85 | | | RRT 0.89 | | | RRT 1.00 | | |
| F01 | 0.12 | ± | 0.01 | 0.27 | ± | 0.02 | 2.06 | ± | 0.01 | 97.56 | ± | 0.03 |
| F02 | 0.13 | ± | 0.00 | 0.31 | ± | 0.00 | 2.89 | ± | 0.02 | 96.67 | ± | 0.02 |
| F03 | 0.23 | ± | 0.00 | 0.37 | ± | 0.02 | 2.86 | ± | 0.02 | 96.54 | ± | 0.04 |
| F04 | 0.14 | ± | 0.00 | 0.29 | ± | 0.00 | 2.38 | ± | 0.01 | 97.20 | ± | 0.01 |
| F05 | 0.80 | ± | 0.00 | 0.27 | ± | 0.01 | 1.86 | ± | 0.02 | 97.08 | ± | 0.02 |
| F06 | 0.44 | ± | 0.00 | 0.32 | ± | 0.02 | 2.06 | ± | 0.01 | 97.17 | ± | 0.03 |
| F07 | n/a | ± | n/a | 0.21 | ± | 0.05 | 1.86 | ± | 0.03 | 97.93 | ± | 0.08 |
| F08 | n/a | ± | n/a | 0.16 | ± | 0.01 | 2.33 | ± | 0.02 | 97.50 | ± | 0.02 |
| F09 | 0.04 | ± | 0.00 | 0.27 | ± | 0.01 | 2.34 | ± | 0.03 | 97.35 | ± | 0.02 |
| F10 | 0.00 | ± | 0.00 | 0.14 | ± | 0.01 | 1.86 | ± | 0.01 | 98.00 | ± | 0.02 |
| F11 | 0.08 | ± | 0.00 | 0.29 | ± | 0.02 | 2.07 | ± | 0.02 | 97.56 | ± | 0.02 |
| F12 | n/a | ± | n/a | 0.24 | ± | 0.00 | 2.16 | ± | 0.01 | 97.60 | ± | 0.01 |
| F13 | n/a | ± | n/a | 0.10 | ± | 0.01 | 1.69 | ± | 0.01 | 98.21 | ± | 0.00 |
| F14 | 0.05 | ± | 0.00 | 0.24 | ± | 0.02 | 1.77 | ± | 0.00 | 97.94 | ± | 0.01 |
| F15 | 0.04 | ± | 0.00 | 0.35 | ± | 0.02 | 2.42 | ± | 0.01 | 97.19 | ± | 0.01 |
| F16 | 0.07 | ± | 0.00 | 0.41 | ± | 0.02 | 2.57 | ± | 0.02 | 96.95 | ± | 0.01 |

TABLE 17
Percentage of peak areas by SEC for formulations in Study
1.2 at t2 (two weeks at 25° C.)
LB141 S1.2 t2 SEC

| | HMW2 | | | HMW1 | | | monomer | | |
|---|---|---|---|---|---|---|---|---|---|
| Sample | RRT 0.85 | | | RRT 0.89 | | | RRT 1.00 | | |
| F01 | 0.22 | ± | 0.02 | 1.71 | ± | 0.01 | 98.07 | ± | 0.01 |
| F02 | 0.29 | ± | 0.01 | 2.28 | ± | 0.01 | 97.43 | ± | 0.01 |
| F03 | 0.30 | ± | 0.02 | 2.16 | ± | 0.02 | 97.54 | ± | 0.00 |
| F04 | 0.24 | ± | 0.01 | 1.98 | ± | 0.01 | 97.78 | ± | 0.01 |
| F05 | 0.24 | ± | 0.03 | 1.63 | ± | 0.03 | 98.13 | ± | 0.00 |
| F06 | 0.31 | ± | 0.02 | 1.91 | ± | 0.05 | 97.77 | ± | 0.07 |
| F07 | 0.27 | ± | 0.00 | 1.69 | ± | 0.01 | 98.04 | ± | 0.01 |
| F08 | 0.24 | ± | 0.01 | 1.90 | ± | 0.01 | 97.86 | ± | 0.00 |
| F09 | 0.23 | ± | 0.01 | 1.97 | ± | 0.00 | 97.81 | ± | 0.01 |
| F10 | 0.13 | ± | 0.02 | 1.65 | ± | 0.03 | 98.23 | ± | 0.01 |
| F11 | 0.16 | ± | 0.01 | 1.74 | ± | 0.01 | 98.10 | ± | 0.01 |
| F12 | 0.37 | ± | 0.00 | 1.99 | ± | 0.00 | 97.64 | ± | 0.00 |
| F13 | 0.18 | ± | 0.01 | 1.51 | ± | 0.02 | 98.31 | ± | 0.01 |
| F14 | 0.19 | ± | 0.01 | 1.51 | ± | 0.01 | 98.30 | ± | 0.01 |
| F15 | 0.32 | ± | 0.03 | 2.06 | ± | 0.04 | 97.63 | ± | 0.01 |
| F16 | 0.30 | ± | 0.02 | 2.22 | ± | 0.01 | 97.48 | ± | 0.03 |

Of the most stable formulations at t2 (formulations, F01, F05, F11, F13, and F14), all contain citrate as the buffer and mannitol as the stabilizer (Table 17). The only degradation product detected by SEC is the higher MW species having a RRT of 0.85, indicating that, at 25° C., the aggregation was not being forced to higher MW species. After four weeks at 25° C. (t4), the monomer content is still quite high, with many formulations retaining 98% or more (Table 18). A new column was introduced, likely resulting in the slightly higher monomer levels seen in Table 18, including small variations in the amount of the aggregate peak at RRT 0.85. The lowest values for monomer content are for formulations that contain succinate at pH 5.5, suggesting that the optimal pH is greater than 5.5.

TABLE 18
Percentage of peak areas by SEC for formulations
in Study 1.2 at t4 (four weeks at 25° C.)
LB141 S1.2 t4 SEC

| | HMW2 | | | HMW1 | | | monomer | | |
|---|---|---|---|---|---|---|---|---|---|
| Sample | RRT 0.85 | | | RRT 0.89 | | | RRT 1.00 | | |
| F01 | 0.13 | ± | 0.01 | 2.15 | ± | 0.02 | 97.71 | ± | 0.05 |
| F02 | 0.12 | ± | 0.00 | 2.93 | ± | 0.03 | 96.95 | ± | 0.03 |
| F03 | 0.19 | ± | 0.00 | 2.72 | ± | 0.00 | 97.09 | ± | 0.01 |
| F04 | 0.15 | ± | 0.01 | 2.62 | ± | 0.01 | 97.24 | ± | 0.01 |
| F05 | 0.54 | ± | 0.00 | 1.04 | ± | 0.01 | 98.37 | ± | 0.04 |
| F06 | 0.32 | ± | 0.01 | 1.42 | ± | 0.03 | 98.25 | ± | 0.05 |
| F07 | 0.01 | ± | 0.00 | 1.50 | ± | 0.00 | 98.50 | ± | 0.00 |
| F08 | n/a | ± | n/a | 0.72 | ± | 0.01 | 99.28 | ± | 0.01 |
| F09 | n/a | ± | n/a | 0.58 | ± | 0.01 | 99.42 | ± | 0.01 |
| F10 | 0.00 | ± | 0.00 | 1.54 | ± | 0.02 | 98.46 | ± | 0.02 |
| F11 | n/a | ± | n/a | 0.22 | ± | 0.04 | 99.78 | ± | 0.04 |
| F12 | n/a | ± | n/a | 0.71 | ± | 0.01 | 99.29 | ± | 0.01 |
| F13 | 0.00 | ± | 0.00 | 1.28 | ± | 0.02 | 98.72 | ± | 0.02 |
| F14 | n/a | ± | n/a | 0.33 | ± | 0.02 | 99.67 | ± | 0.02 |
| F15 | 0.02 | ± | 0.00 | 0.70 | ± | 0.04 | 99.28 | ± | 0.03 |
| F16 | 0.05 | ± | 0.01 | 0.73 | ± | 0.01 | 99.22 | ± | 0.01 |

TABLE 19
Purity by RP HPLC for formulations
in Study 1.2 at t0
LB141 S1.2 t0 RP

| Sample | monomer | | |
|---|---|---|---|
| F01 | 88.07 | ± | 0.87 |
| F02 | 87.08 | ± | 0.53 |
| F03 | 87.25 | ± | 0.26 |
| F04 | 86.95 | ± | 0.40 |
| F05 | 87.24 | ± | 0.47 |
| F06 | 87.75 | ± | 0.20 |
| F07 | 87.04 | ± | 0.10 |
| F08 | 87.16 | ± | 0.14 |
| F09 | 87.39 | ± | 0.25 |
| F10 | 88.28 | ± | 0.22 |
| F11 | 88.82 | ± | 0.11 |
| F12 | 86.59 | ± | 0.33 |
| F13 | 89.27 | ± | 0.34 |
| F14 | 89.61 | ± | 0.05 |
| F15 | 88.45 | ± | 0.16 |
| F16 | 87.34 | ± | 0.30 |

The purity by RP HPLC at t0 is found in Table 19. All of the formulations display purities in the 87-89% range. After storage at 40° C. for one week, the purity decreases for many of the formulations, some to less than 80% (Table 20). A few (formulations F05, F14, F15, and F16) continue to exhibit purities near 85%. The general trend seems to be better stability at lower protein concentrations and with higher amounts of mannitol.

TABLE 20
Purity by RP HPLC for formulations
in Study 1.2 at t1 (one week at 40° C.)
LB141 S1.2 t1 1 wk 40 C. RP

| Sample | monomer | | |
|---|---|---|---|
| F01 | 81.32 | ± | 0.58 |
| F02 | 80.61 | ± | 0.54 |
| F03 | 81.95 | ± | 0.18 |
| F04 | 79.06 | ± | 0.24 |
| F05 | 85.17 | ± | 0.64 |
| F06 | 82.92 | ± | 0.20 |
| F07 | 80.26 | ± | 0.12 |
| F08 | 80.73 | ± | 0.39 |
| F09 | 82.51 | ± | 0.71 |
| F10 | 79.34 | ± | 0.62 |
| F11 | 84.37 | ± | 0.77 |
| F12 | 75.09 | ± | 0.52 |
| F13 | 80.36 | ± | 0.29 |
| F14 | 86.44 | ± | 0.26 |
| F15 | 85.11 | ± | 0.62 |
| F16 | 84.32 | ± | 0.57 |

TABLE 21
Purity by RP HPLC for formulations in Study
1.2 at t2 (two weeks at 25° C.)
LB141 S1.2 t2 2 wk 25 C. RP

| Sample | monomer | | |
|---|---|---|---|
| F01 | 83.84 | ± | 0.61 |
| F02 | 83.16 | ± | 0.57 |
| F03 | 84.86 | ± | 0.46 |
| F04 | 84.90 | ± | 0.25 |
| F05 | 86.88 | ± | 0.16 |
| F06 | 86.51 | ± | 0.26 |
| F07 | 85.91 | ± | 0.49 |
| F08 | 85.71 | ± | 0.36 |
| F09 | 84.90 | ± | 0.31 |
| F10 | 85.91 | ± | 0.18 |
| F11 | 87.00 | ± | 0.34 |
| F12 | 84.96 | ± | 0.25 |
| F13 | 86.15 | ± | 0.15 |
| F14 | 87.93 | ± | 0.16 |
| F15 | 86.92 | ± | 0.24 |
| F16 | 86.84 | ± | 0.79 |

At t2, the purity changed very little for most formulations, possibly not at all, given the error in this method (Table 21). After four weeks at 25° C. (t4), there is some wider range of purities observed (Table 22). The least stable formulations are either at lower pH (5.5) or have the highest citrate concentration. The most stable formulations contain mannitol as the stabilizer or use succinate at the highest concentration. In general, the main trend is for greater stability at lower protein concentrations.

TABLE 22
Purity by RP HPLC for formulations in Study
1.2 at t4 (four weeks at 25° C.)
LB141 S1.2 t4 4 wk 25 C. RP

| Sample | monomer | | |
|---|---|---|---|
| F01 | 80.13 | ± | 1.49 |
| F02 | 79.73 | ± | 2.64 |
| F03 | 84.19 | ± | 0.37 |
| F04 | 81.17 | ± | 0.11 |
| F05 | 87.97 | ± | 0.33 |
| F06 | 88.21 | ± | 0.16 |
| F07 | 86.35 | ± | 0.46 |
| F08 | 84.16 | ± | 0.39 |
| F09 | 87.54 | ± | 0.53 |
| F10 | 82.49 | ± | 0.15 |
| F11 | 84.54 | ± | 0.26 |
| F12 | 77.29 | ± | 0.30 |
| F13 | 81.07 | ± | 0.71 |
| F14 | 87.67 | ± | 0.14 |
| F15 | 86.35 | ± | 0.31 |
| F16 | 86.47 | ± | 0.34 |

TABLE 23
Relative intensities of the cIEF peaks
for formulations F01-F04 in Study 1.2

| | | t0 | | t1 1 week 40° C. | | t2 2 weeks 25° C. | | t4 4 weeks 25° C. | |
|---|---|---|---|---|---|---|---|---|---|
| Form | Peak | pI | Rel. Area | pI | Rel. Area | pI | Rel. Area | pI | Rel. Area |
| F01 | 1 | 5.81 | 1.30 | 5.89 | 0.56 | 5.88 | 0.95 | 5.87 | 1.27 |
| | 2 | 5.76 | 52.48 | 5.85 | 45.29 | 5.82 | 45.57 | 5.80 | 48.45 |
| | 3 | 5.73 | 16.30 | 5.81 | 19.63 | 5.79 | 22.26 | 5.78 | 22.05 |
| | 4,5 | 5.69 | 19.28 | 5.77 | 24.87 | 5.74 | 21.77 | 5.73 | 20.29 |
| | 6 | 5.64 | 7.65 | 5.71 | 7.66 | 5.69 | 7.72 | 5.69 | 6.82 |
| | 7 | 5.61 | 2.99 | 5.67 | 1.98 | 5.65 | 1.72 | 5.65 | 1.12 |
| | 8 | | | | | | | | |
| F02 | 1 | 5.81 | 1.51 | 5.88 | 0.68 | 5.85 | 1.64 | 5.86 | 1.52 |
| | 2 | 5.76 | 49.87 | 5.84 | 42.77 | 5.79 | 46.60 | 5.80 | 47.88 |
| | 3 | 5.73 | 21.19 | 5.81 | 20.99 | 5.76 | 25.42 | 5.78 | 26.30 |
| | 4,5 | 5.68 | 18.96 | 5.77 | 27.64 | 5.70 | 16.71 | 5.72 | 17.87 |
| | 6 | 5.64 | 6.73 | 5.71 | 6.76 | 5.66 | 8.12 | 5.68 | 5.84 |
| | 7 | 5.60 | 1.74 | 5.68 | 1.16 | 5.62 | 1.50 | 5.64 | 0.58 |
| | 8 | | | | | | | | |
| F03 | 1 | 5.83 | 1.66 | 5.89 | 0.59 | 5.82 | 1.49 | 5.88 | 0.90 |
| | 2 | 5.77 | 58.93 | 5.84 | 46.19 | 5.76 | 51.08 | 5.82 | 52.86 |
| | 3 | 5.74 | 16.39 | 5.81 | 16.03 | 5.72 | 21.60 | 5.79 | 19.42 |
| | 4,5 | 5.70 | 15.84 | 5.77 | 26.72 | 5.67 | 17.64 | 5.74 | 19.93 |
| | 6 | 5.66 | 5.77 | 5.71 | 8.61 | 5.62 | 7.20 | 5.69 | 5.94 |
| | 7 | 5.62 | 1.41 | 5.68 | 1.86 | 5.58 | 0.99 | 5.65 | 0.95 |
| | 8 | | | | | | | | |
| F04 | 1 | 5.82 | 1.76 | 5.88 | 2.43 | 5.86 | 1.31 | 5.87 | 1.31 |
| | 2 | 5.76 | 53.50 | 5.84 | 44.17 | 5.79 | 47.19 | 5.81 | 50.44 |
| | 3 | 5.74 | 19.12 | 5.80 | 25.77 | 5.77 | 23.37 | 5.78 | 19.06 |
| | 4,5 | 5.69 | 19.26 | 5.74 | 18.67 | 5.72 | 19.40 | 5.73 | 20.67 |
| | 6 | 5.65 | 4.85 | 5.70 | 7.50 | 5.67 | 7.17 | 5.68 | 6.90 |
| | 7 | 5.60 | 1.51 | 5.65 | 1.45 | 5.63 | 1.57 | 5.64 | 1.62 |
| | 8 | | | | | | | | |
| F05 | 1 | 5.81 | 1.46 | 5.88 | 2.98 | 5.86 | 1.47 | 5.87 | 1.49 |
| | 2 | 5.75 | 57.36 | 5.83 | 41.56 | 5.80 | 49.21 | 5.81 | 53.38 |
| | 3 | 5.73 | 19.00 | 5.80 | 32.18 | 5.77 | 24.92 | 5.78 | 22.57 |
| | 4,5 | 5.68 | 15.23 | 5.75 | 18.42 | 5.73 | 17.86 | 5.73 | 16.70 |
| | 6 | 5.64 | 5.85 | 5.70 | 4.45 | 5.68 | 6.00 | 5.69 | 5.10 |
| | 7 | 5.60 | 1.11 | 5.67 | 0.43 | 5.64 | 0.54 | 5.65 | 0.75 |
| | 8 | | | | | | | | |
| F06 | 1 | 5.87 | 1.01 | 5.87 | 1.41 | 5.88 | 1.07 | 5.86 | 1.39 |
| | 2 | 5.82 | 44.28 | 5.83 | 41.42 | 5.82 | 54.42 | 5.81 | 51.10 |
| | 3 | 5.79 | 20.35 | 5.80 | 30.70 | 5.79 | 20.06 | 5.77 | 24.61 |
| | 4,5 | 5.75 | 24.16 | 5.75 | 18.40 | 5.75 | 18.29 | 5.72 | 16.23 |
| | 6 | 5.70 | 8.40 | 5.70 | 6.16 | 5.70 | 5.66 | 5.67 | 5.57 |
| | 7 | 5.66 | 1.79 | 5.66 | 1.91 | 5.66 | 0.49 | 5.63 | 1.10 |
| | 8 | | | | | | | | |
| F07 | 1 | 5.83 | 1.28 | 5.86 | 1.07 | 5.88 | 1.21 | 5.87 | 1.25 |
| | 2 | 5.76 | 53.15 | 5.81 | 45.54 | 5.82 | 51.93 | 5.82 | 55.24 |
| | 3 | 5.74 | 19.78 | 5.78 | 26.26 | 5.79 | 19.13 | 5.79 | 17.92 |
| | 4,5 | 5.69 | 18.34 | 5.73 | 20.31 | 5.74 | 19.19 | 5.74 | 19.77 |
| | 6 | 5.65 | 6.23 | 5.69 | 6.81 | 5.69 | 7.44 | 5.70 | 5.12 |
| | 7 | 5.61 | 1.21 | 5.65 | 1.13 | 5.65 | 1.11 | 5.66 | 0.71 |
| | 8 | | | | | | | | |
| F08 | 1 | 5.82 | 1.82 | 5.90 | 0.64 | 5.89 | 0.98 | 5.88 | 1.11 |
| | 2 | 5.76 | 50.43 | 5.85 | 44.58 | 5.85 | 36.24 | 5.82 | 55.94 |
| | 3 | 5.73 | 18.50 | 5.82 | 15.97 | 5.81 | 31.91 | 5.79 | 19.31 |
| | 4,5 | 5.69 | 18.82 | 5.78 | 27.87 | 5.75 | 22.18 | 5.75 | 18.00 |
| | 6 | 5.64 | 7.46 | 5.72 | 8.63 | 5.70 | 6.61 | 5.70 | 5.27 |
| | 7 | 5.61 | 2.97 | 5.69 | 2.31 | 5.66 | 2.08 | 5.66 | 0.38 |
| | 8 | | | | | | | | |
| F09 | 1 | 5.82 | 1.44 | 5.89 | 0.93 | 5.84 | 1.88 | 5.90 | 1.34 |
| | 2 | 5.76 | 49.53 | 5.85 | 48.76 | 5.78 | 50.99 | 5.85 | 51.78 |
| | 3 | 5.73 | 23.28 | 5.81 | 22.91 | 5.75 | 24.45 | 5.82 | 24.61 |
| | 4,5 | 5.68 | 17.73 | 5.77 | 19.07 | 5.70 | 15.62 | 5.76 | 17.64 |
| | 6 | 5.64 | 6.58 | 5.72 | 7.54 | 5.65 | 5.00 | 5.71 | 4.28 |
| | 7 | 5.60 | 1.44 | 5.68 | 0.79 | 5.61 | 2.08 | 5.67 | 0.36 |
| | 8 | | | | | | | | |
| F10 | 1 | 5.81 | 1.40 | 5.90 | 1.10 | 5.86 | 1.68 | 5.88 | 0.82 |
| | 2 | 5.76 | 50.54 | 5.85 | 47.64 | 5.80 | 47.05 | 5.83 | 51.54 |
| | 3 | 5.73 | 20.27 | 5.81 | 21.21 | 5.77 | 24.93 | 5.80 | 18.82 |
| | 4,5 | 5.68 | 19.32 | 5.77 | 21.64 | 5.72 | 18.72 | 5.75 | 20.56 |
| | 6 | 5.64 | 6.93 | 5.71 | 6.98 | 5.67 | 7.01 | 5.71 | 6.89 |
| | 7 | 5.60 | 1.54 | 5.67 | 1.44 | 5.63 | 0.62 | 5.67 | 1.37 |
| | 8 | | | | | | | | |
| F11 | 1 | 5.84 | 2.37 | 5.89 | 0.99 | 5.86 | 1.37 | 5.91 | 0.76 |
| | 2 | 5.78 | 51.88 | 5.85 | 49.39 | 5.80 | 49.13 | 5.85 | 53.52 |
| | 3 | 5.75 | 18.80 | 5.81 | 20.70 | 5.77 | 25.37 | 5.82 | 17.09 |
| | 4,5 | 5.70 | 18.84 | 5.77 | 20.99 | 5.72 | 18.36 | 5.77 | 21.27 |
| | 6 | 5.66 | 7.24 | 5.72 | 6.40 | 5.68 | 5.01 | 5.72 | 6.25 |
| | 7 | 5.61 | 0.88 | 5.68 | 1.53 | 5.64 | 0.75 | 5.68 | 1.11 |
| | 8 | | | | | | | | |
| F12 | 1 | 5.88 | 1.39 | 5.89 | 0.80 | 5.87 | 1.21 | 5.89 | 0.72 |
| | 2 | 5.82 | 43.40 | 5.85 | 49.65 | 5.81 | 47.83 | 5.84 | 48.09 |
| | 3 | 5.80 | 22.44 | 5.82 | 19.51 | 5.78 | 22.35 | 5.81 | 18.78 |
| | 4,5 | 5.75 | 24.14 | 5.77 | 22.45 | 5.73 | 20.82 | 5.76 | 22.81 |
| | 6 | 5.70 | 8.21 | 5.72 | 6.93 | 5.68 | 6.56 | 5.72 | 7.87 |
| | 7 | 5.66 | 0.42 | 5.68 | 0.67 | 5.64 | 1.23 | 5.68 | 1.73 |
| | 8 | | | | | | | | |
| F13 | 1 | 5.84 | 1.46 | 5.86 | 1.44 | 5.84 | 1.77 | 5.89 | 0.73 |
| | 2 | 5.77 | 51.26 | 5.81 | 54.06 | 5.78 | 45.89 | 5.85 | 30.54 |
| | 3 | 5.75 | 20.04 | 5.78 | 15.96 | 5.75 | 27.27 | 5.81 | 35.06 |
| | 4,5 | 5.70 | 19.86 | 5.74 | 20.99 | 5.70 | 16.92 | 5.76 | 23.90 |
| | 6 | 5.66 | 6.41 | 5.69 | 6.94 | 5.65 | 7.07 | 5.71 | 8.08 |
| | 7 | 5.62 | 0.98 | 5.65 | 0.60 | 5.61 | 1.08 | 5.67 | 1.68 |
| | 8 | | | | | | | | |
| F14 | 1 | 5.85 | 1.16 | 5.87 | 1.22 | 5.84 | 1.95 | 5.89 | 0.99 |
| | 2 | 5.78 | 49.23 | 5.82 | 49.91 | 5.78 | 48.22 | 5.83 | 51.40 |
| | 3 | 5.76 | 17.44 | 5.79 | 23.66 | 5.76 | 26.77 | 5.80 | 16.47 |
| | 4,5 | 5.72 | 21.47 | 5.75 | 18.95 | 5.70 | 15.61 | 5.76 | 22.78 |
| | 6 | 5.67 | 7.74 | 5.70 | 4.73 | 5.66 | 6.12 | 5.71 | 6.90 |
| | 7 | 5.63 | 2.96 | 5.67 | 1.54 | 5.62 | 1.33 | 5.67 | 1.45 |
| | 8 | | | | | | | | |
| F15 | 1 | 5.86 | 1.01 | 5.89 | 0.77 | 5.86 | 1.34 | 5.88 | 0.51 |
| | 2 | 5.79 | 45.27 | 5.85 | 45.84 | 5.80 | 50.91 | 5.84 | 51.38 |
| | 3 | 5.77 | 20.51 | 5.81 | 22.94 | 5.77 | 24.33 | 5.80 | 16.84 |
| | 4,5 | 5.73 | 21.61 | 5.77 | 21.53 | 5.72 | 18.71 | 5.76 | 23.89 |
| | 6 | 5.68 | 9.86 | 5.72 | 7.57 | 5.67 | 4.38 | 5.72 | 5.70 |
| | 7 | 5.64 | 1.74 | 5.69 | 1.35 | 5.63 | 0.34 | 5.68 | 1.68 |
| | 8 | | | | | | | | |
| F16 | 1 | 5.83 | 1.61 | 5.91 | 1.07 | 5.87 | 1.26 | 5.88 | 0.86 |
| | 2 | 5.77 | 52.78 | 5.86 | 47.89 | 5.82 | 41.42 | 5.84 | 47.18 |
| | 3 | 5.75 | 23.24 | 5.83 | 19.38 | 5.79 | 32.26 | 5.80 | 15.97 |
| | 4,5 | 5.70 | 15.76 | 5.79 | 19.69 | 5.72 | 18.09 | 5.76 | 24.04 |
| | 6 | 5.65 | 5.48 | 5.74 | 9.56 | 5.67 | 5.89 | 5.72 | 9.29 |
| | 7 | 5.61 | 1.13 | 5.70 | 2.41 | 5.63 | 1.07 | 5.68 | 2.67 |
| | 8 | | | | | | | | |

PLS modeling can provide some guidance as to the most effective excipients. This modeling provides global fits to a limited number of data points; however, it can provide useful guidance in terms of identifying trends that may be obscured by noise in the data, especially when the data sets are considered as a whole.

A PLS1 model for Study 1.2 was constructed using the difference in monomer content at t1 as the endpoint. PLS is a quadratic model that also included pH-buffer interactions terms. The model employed 8 principal components (PCs) and had a correlation coefficient of 0.998 for the calibration set and 0.946 for the validation set, indicating it was a very accurate and robust model. The correlation coefficients for the various factors are summarized in Table 24. Since one is trying to minimize the difference, stabilizers will have a negative correlation coefficient.

TABLE 24
Correlation coefficients for the factors used in constructing
the PLS1 model 1 using the difference in monomer content
at t1 as the endpoint. Factors determined to be
statistically significant (p < 0.05) are shown in bold type.

| Factor | r-value |
|---|---|
| pH | −0.704 |
| prot | +0.160 |
| citrate | −0.602 |
| succinate | +0.296 |
| octanoate | −0.095 |
| mannitol | +0.037 |
| trehalose | +0.004 |

A response surface showing the effect of pH and citrate shows the optimal pH to be ˜6. While citrate was found to be beneficial, the optimal concentration was near 10 mM, although this is a very shallow minimum. The response surface for the effect of pH and succinate is more complex. At pH 6, succinate is a stabilizer, at least at 20 mM, but the effect is the same as if there were no succinate present. The last surface presented shows that octanoate appears to be a weak stabilizer at pH 6, with a shallow minimum near 10 mM. Note that octanoate was not determined to have significant effect on stability, and the response surfaces reveal relatively shallow profiles.

A PLS2 model was constructed using two different endpoints, the monomer content at t2 and at t4. The model only used 1 PC and had a correlation coefficient of 0.861 for the calibration set and 0.698 for the validation set, indicating it was not nearly as accurate as the previous model. The correlation coefficients for the various factors are listed in Table 25. Citrate is shown to be a weak stabilizer, while succinate is listed as a destabilizer. The values in Table 25 do not reflect the quadratic or interaction terms, so an examination of the actual response surfaces is helpful.

TABLE 25
Correlation coefficients for the factors used in constructing the
PLS2 model using the monomer contents at t2 and t4 (two and
four weeks at 25° C.) as the endpoints. Factors determined to
be statistically significant (p < 0.05) are shown in bold type.

| Factor | r-value |
|---|---|
| pH | +0.024 |
| prot | −0.093 |
| citrate | +0.087 |
| succinate | −0.149 |
| octanoate | −0.070 |
| mannitol | +0.065 |
| trehalose | −0.126 |
| PS 80 | −0.165 |

The response surface for the effect of pH and citrate indicates that the optimal pH is from 5.5 to 5.7. The optimal citrate concentration is near 10 mM, according to this model, but the range over the entire surface is quite small (<0.2%), so any variations may not be significant. Octanoate is a destabilizer according to this model, especially above a concentration of 10 mM. Similarly, PS 80 decreases the stability of albiglutide at 25° C. according to this model. In addition, both models find that increased protein concentration leads to lower stability, although the effect is not large. Note that this model is of lower quality and may not agree with findings from other work done in Study 1.

A PLS1 model using just the monomer content at t4 as the endpoint was constructed. The model employed 8 PCs and had a correlation coefficient of 0.999 for the calibration set and 0.856 for the validation set, making it a high quality model, comparable to the first PLS1 model discussed above. The correlation coefficients were sizable for many of the factors, but only three, pH, protein, and succinate, were deemed to be significant (Table 26).

TABLE 26
Correlation coefficients for the factors used in
constructing the PLS1 model using the monomer contents
at t4 (four weeks at 25° C.) as the endpoint. Factors
determined to be statistically significant (p < 0.05)
are shown in bold type.

| Factor | r-value |
|---|---|
| pH | +0.585 |
| prot | −0.137 |
| citrate | +0.295 |
| succinate | −0.497 |
| octanoate | −0.261 |
| mannitol | −0.025 |
| trehalose | −0.154 |
| PS 80 | −0.007 |

The response surface for the effect of pH and citrate shows the pH optimum to be near pH 6.0, suggesting that the t2 data was influencing any indication of a lower pH being superior. The model indicates that the optimal citrate concentration was 10 mM.

The effect of protein concentration is of interest, as one could effectively extend the shelf-life of the product if there is a pronounced concentration effect. This model indicates that the stability decreases with increasing protein concentration, especially once the concentration increases above 60 mg/mL. The model indicates that the protein concentration does have a significant effect on monomer content at t4.

Succinate is predicted to have a non-linear effect at pH 6, although the effect at that pH is very small. Meanwhile, it is clearly destabilizing at pH 5.5. Given the small impact of succinate, it probably best to consider citrate as the best selection for the buffer, based on all of the Study 1.2 data. Finally, octanoate is also destabilizing, but in a more much pH-independent fashion that succinate. Theoptimal pH is predicted to be near 6.0.

A fourth PLS (PLS1) model was constructed using the difference in purity by RP HPLC at t1 as the endpoint. The model employed 3 PCs and had a correlation coefficient of 0.949 for the calibration set and 0.748 for the validation set. The correlation coefficients for the factors in this model are summarized in Table 27. Three factors were calculated to be significant, pH, citrate and octanoate.

TABLE 27
Correlation coefficients for the factors used
in constructing the PLS1 model using the difference in
purity by RP HPLC at t1 (one week at 40° C.) as
the endpoint. Factors determined to be statistically significant
(p < 0.05) are shown in bold type.

| Factor | r-value |
|---|---|
| pH | +0.252 |
| prot | −0.052 |
| citrate | +0.217 |
| succinate | −0.080 |
| octanoate | +0.372 |
| mannitol | +0.029 |
| trehalose | +0.071 |
| PS 80 | −0.059 |

This PLS model indicates that the optimal pH is near 5.5 to 5.7 if citrate concentrations above 10 mM. As seen before, the response surface for pH and succinate is quite complex. It shows that, with no succinate present, the optimal pH is 5.5. However, with succinate present, the trend is reversed and pH 6 is more favorable and succinate providing some increased stability at 20 mM. Octanoate is destabilizing at all pH values according to this model. The model also shows PS 80 to be beneficial for maintaining purity with the optimal pH near 5.5 to 5.7.

Overall, Study 1.2 provides some important information on the stabilization of albiglutide. The optimal pH is near 6.0, although slightly lower pH values do not alter the stability profile appreciably. The data on buffer effects is varied, but the general sense is that a modest amount of citrate, about 10 to 15 mM, is beneficial. The effects of succinate are more difficult to decipher, but there is no data to suggest that it is as good a stabilizer as citrate.

The stability decreases as the protein concentration increases, especially in terms of the physical stability measured by SEC. Concentrations above 70 mg/mL or so do reduce the stability by some degree. While octanoate was shown to be detrimental to stability, some PS 80 appears to provide some benefit, but rarely enough to be considered significant in any of the mathematical models.

Of the two structural stabilizers considered in Study 1.2, mannitol and trehalose, there does not seem to be any significant difference between the two. Some of the data suggest that mannitol would be a better stabilizer, but the differences are so slight that this cannot be said conclusively. It appears that the impact of pH, buffer and protein concentration are more important.

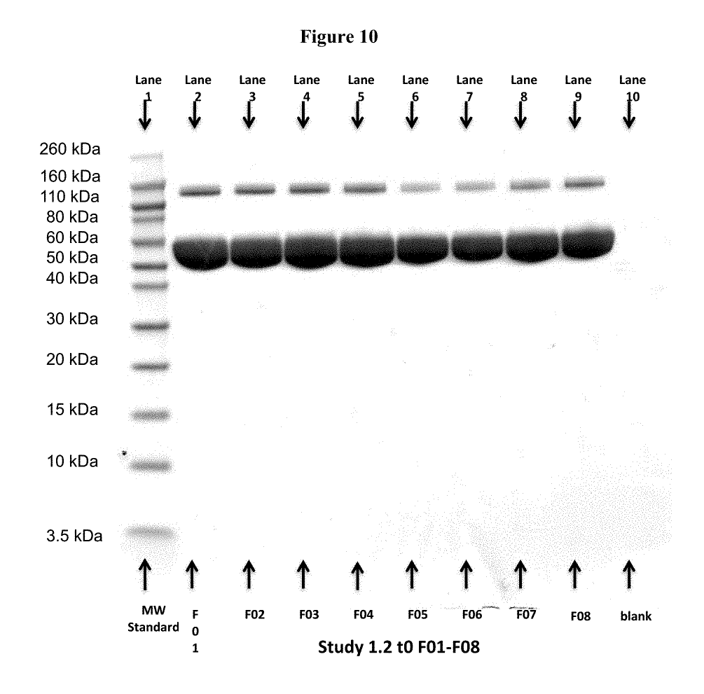

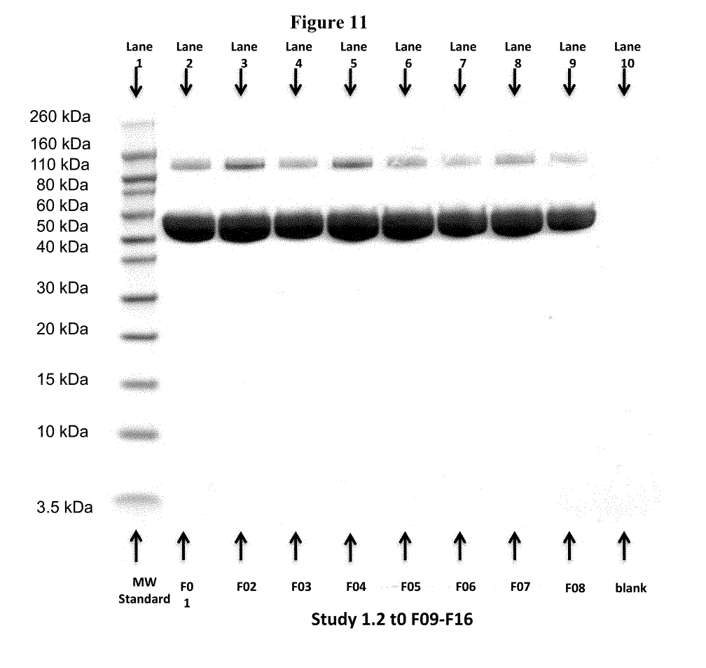

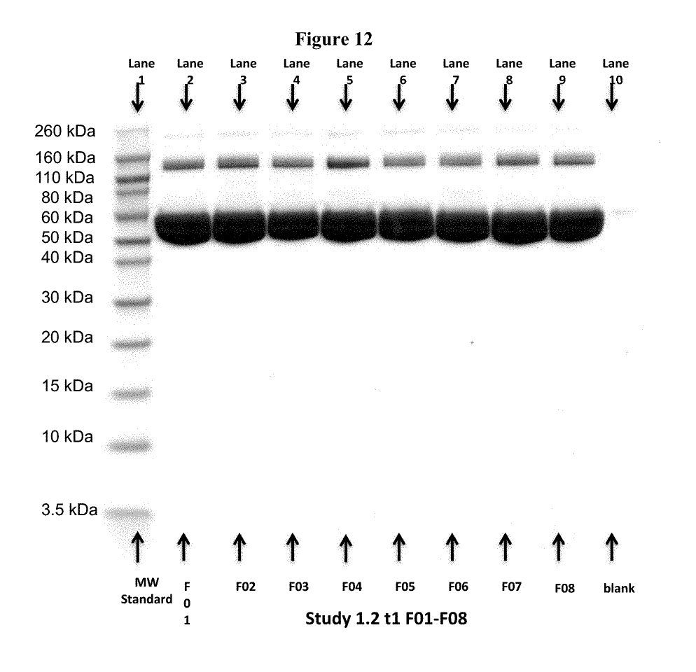

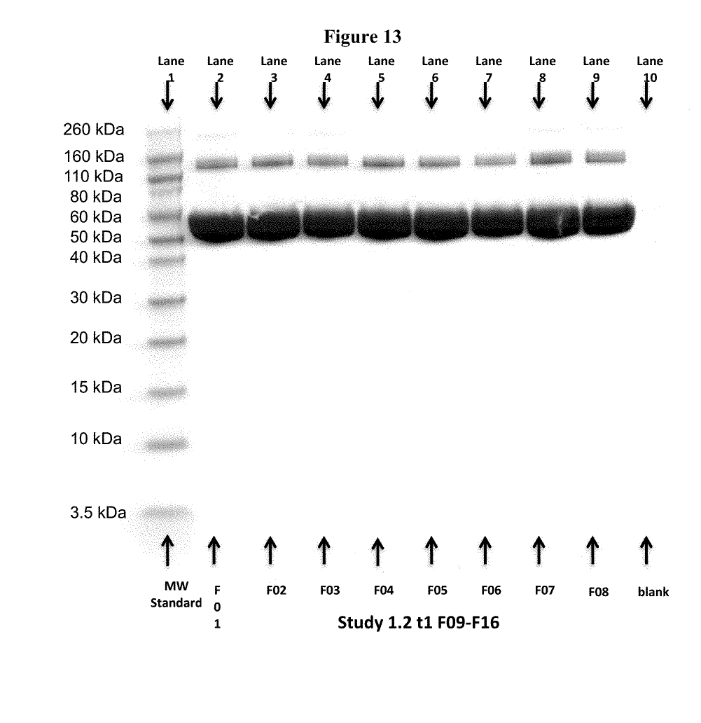

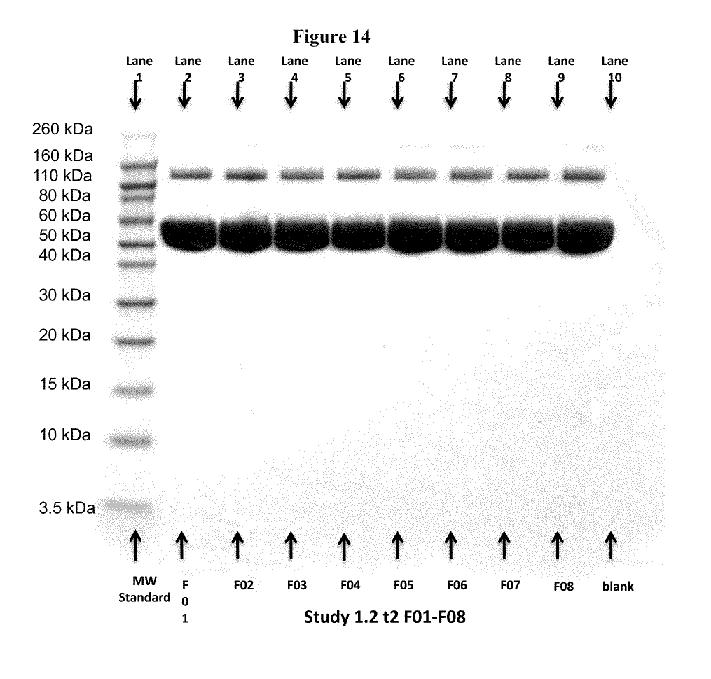

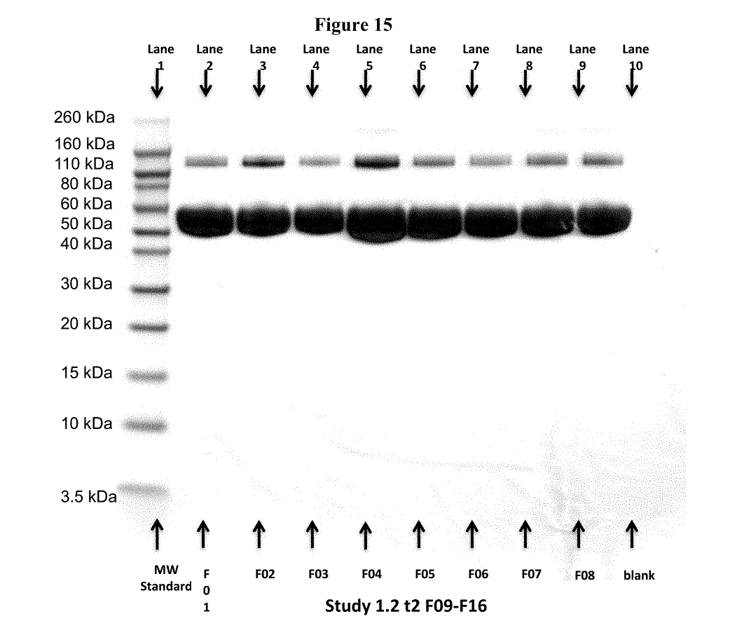

Finally, these samples were examined using non-reduced SDS-PAGE. At t0, there is the main monomer band near 50 kD and a second weaker band near 110 kD, which is presumably the dimer (FIGS. 10 and 11). At t1, a faint band near 200 kD appears in some of the formulations (FIGS. 12 and 13). This band does not appear in the t2 samples (FIGS. 14 and 15). These results mirror the SEC findings, where 25° C. does not generate the species with RRT 0.78, while 40° C. treatment does.

### Study 1.3.

The final study was designed to investigate concentration effects of both trehalose and arginine HCl (Table 28). Citrate was the only buffer system investigated, and was varied between 5 and 15 mM. Protein concentrations were taken as low as 30 mg/mL, as the previous data suggested that the use of lower concentrations may extend the shelf life. The pH was fixed at 6.0 for all except two samples. Octanoate was discontinued as a potential stabilizer based on previous data (Study 1.2 and Study 2.0). Samples were kept for a period of five months.

TABLE 28
Composition of Study 1.3 formulations

| | | | protein | | | |
|---|---|---|---|---|---|---|
| Form | | citrate | HCl | arginine | trehalose | PS 80 |
| No | pH | mM | mg/mL | mM | mM | wt % |
| 1 | 5.7 | 5 | 50 | 100 | 117 | 0.01 |
| 2 | 5.7 | 10 | 50 | 100 | 117 | 0.01 |
| 3 | 6 | 5 | 30 | 100 | 117 | 0.01 |
| 4 | 6 | 5 | 50 | 100 | 117 | 0.01 |
| 5 | 6 | 10 | 50 | 100 | 117 | 0.01 |
| 6 | 6 | 15 | 50 | 100 | 117 | 0.01 |
| 7 | 6 | 5 | 30 | 50 | 207 | 0.01 |
| 8 | 6 | 5 | 50 | 75 | 162 | 0.01 |
| 9 | 6 | 5 | 80 | 125 | 72 | 0.01 |
| 10 | 6 | 10 | 30 | 50 | 207 | 0.01 |
| 11 | 6 | 10 | 50 | 75 | 162 | 0.01 |
| 12 | 6 | 10 | 80 | 125 | 72 | 0.01 |
| 13 | 6 | 15 | 50 | 100 | 117 | 0.01 |
| 14 | 6 | 5 | 50 | 50 | 207 | 0.01 |
| 15 | 6 | 5 | 50 | 125 | 72 | 0.01 |
| 16 | 6 | 5 | 100 | 100 | 117 | 0 |
| 17 | 6 | 5 | 100 | 100 | 117 | 0.01 |
| 18 | 6 | 5 | 50 | 100 | 117 | 0 |

Samples were assayed at numerous time points and storage temperatures. Stability data were collected after one and two weeks at 40° C., after one, two, or three weeks at 30° C., after four, thirteen and 22 weeks (approximately 1 month, 3 months, and 5 months) at 25° C., and after three and five months at 5° C. Stability testing was performed using SEC and RP HPLC, as summarized below. Conversely, SDS-PAGE and cIEF were discontinued at this point, as they do not appear to be as stability-indicating methods. In addition, samples were assayed at GSK, also using SEC and RP, but adding testing done by cIEF and Caliper analysis.

TABLE 29
pH values for samples from Study 1.3

| | | t1 | t1 | t2 | t2 | t3 | t4 | t13 | t13 | t22 | t22 |
|---|---|---|---|---|---|---|---|---|---|---|---|
| Form | t0 | 25 C. | 40 C. | 25 C. | 40 C. | 25 C. | 25 C. | 5 C. | 25 C. | 5 C. | 25 C. |
| F01 | 5.68 | 5.76 | 5.70 | 5.70 | 5.73 | 5.79 | 5.84 | 5.75 | 5.75 | 5.70 | 5.67 |
| F02 | 5.65 | 5.60 | 5.62 | 5.70 | 5.64 | 5.71 | 5.73 | 5.67 | 5.68 | 5.66 | 5.62 |
| F03 | 6.01 | 5.97 | 6.03 | 6.01 | 6.03 | 6.07 | 6.07 | 6.08 | 6.05 | 5.98 | 6.01 |
| F04 | 6.11 | 6.13 | 6.12 | 6.21 | 6.14 | 6.18 | 6.19 | 6.19 | 6.16 | 6.17 | 6.14 |
| F05 | 5.94 | 5.93 | 5.95 | 6.00 | 5.96 | 6.02 | 6.02 | 5.99 | 5.97 | 5.99 | 5.95 |
| F06 | 5.93 | 5.92 | 6.01 | 5.95 | 5.92 | 5.99 | 5.96 | 5.95 | 5.94 | 5.90 | 5.91 |
| F07 | 6.01 | 6.03 | 6.00 | 6.11 | 6.10 | 6.13 | 6.06 | 6.12 | 6.09 | 6.08 | 6.02 |
| F08 | 6.10 | 6.12 | 6.13 | 6.13 | 6.12 | 6.16 | 6.16 | 6.10 | 6.13 | 6.12 | 6.15 |
| F09 | 6.22 | 6.17 | 6.14 | 6.21 | 6.21 | 6.26 | 6.26 | 6.24 | 6.22 | 6.18 | 6.19 |
| F10 | 5.96 | 5.89 | 5.96 | 5.96 | 5.96 | 5.95 | 5.95 | 5.96 | 5.96 | 5.92 | 5.94 |
| F11 | 5.96 | 5.95 | 5.93 | 5.92 | 5.94 | 6.01 | 5.97 | 5.99 | 5.97 | 5.93 | 5.97 |
| F12 | 6.04 | 6.05 | 6.01 | 6.06 | 6.04 | 6.07 | 6.07 | 6.06 | 6.02 | 6.03 | 6.01 |
| F13 | 6.05 | 5.90 | 5.93 | 5.94 | 5.93 | 5.94 | 5.94 | 5.93 | 5.94 | 5.90 | 5.90 |
| F14 | 6.05 | 5.97 | 6.04 | 6.07 | 6.02 | 6.07 | 5.99 | 6.09 | 6.05 | 6.03 | 6.01 |
| F15 | 6.04 | 6.04 | 6.08 | 6.09 | 6.07 | 6.09 | 6.10 | 6.06 | 6.06 | 6.04 | 6.04 |
| F16 | 6.04 | 5.97 | 5.98 | 6.13 | 6.05 | 6.05 | 6.09 | 6.07 | 6.00 | 6.01 | 6.00 |
| F17 | 6.14 | 6.19 | 6.17 | 6.16 | 6.15 | 6.14 | 6.19 | 6.17 | 6.11 | 6.12 | 6.09 |
| F18 | 5.92 | 5.93 | 5.80 | 5.94 | 5.95 | 5.96 | 5.96 | 5.95 | 6.03 | 5.89 | 5.86 |

TABLE 30
Concentrations by UV for samples from Study 1.3

| | | t1 | t1 | t2 | t2 | t3 | t4 | t13 | t13 | t22 | t22 |
|---|---|---|---|---|---|---|---|---|---|---|---|
| Form | t0 | 25 C. | 40 C. | 25 C. | 40 C. | 25 C. | 25 C. | 5 C. | 25 C. | 5 C. | 25 C. |
| F01 | 57.8 | 52.5 | 51.3 | 51.3 | 51.0 | 50.8 | 51.2 | 51.1 | 53.2 | 51.8 | 50.2 |
| F02 | 48.3 | 48.76 | 49.0 | 49.8 | 49.9 | 49.0 | 48.8 | 49.4 | 49.8 | 55.0 | 48.8 |
| F03 | 30.6 | 30.46 | 31.3 | 31.7 | 31.3 | 30.9 | 30.3 | 31.1 | 31.0 | 30.6 | 31.5 |
| F04 | 50.1 | 48.7 | 48.9 | 49.6 | 49.7 | 49.1 | 48.8 | 48.5 | 50.3 | 49.1 | 50.6 |
| F05 | 50.77 | 49.7 | 50.4 | 49.6 | 49.3 | 48.7 | 49.5 | 49.0 | 44.1 | 52.9 | 51.5 |
| F06 | 51.5 | 50.2 | 49.4 | 50.4 | 51.0 | 52.1 | 50.1 | 48.5 | 49.1 | 49.7 | 50.0 |
| F07 | 29.4 | 29.0 | 30.2 | 30.6 | 30.6 | 28.2 | 29.6 | 30.6 | 29.1 | 29.5 | 29.1 |
| F08 | 47.9 | 47.2 | 49.5 | 47.5 | 48.1 | 47.2 | 47.9 | 47.5 | 50.0 | 46.9 | 48.8 |
| F09 | 79.3 | 78.6 | 79.4 | 77.0 | 79.6 | 78.9 | 77.5 | 75.8 | 78.5 | 79.0 | 78.3 |
| F10 | 31.3 | 29.4 | 33.3 | 29.3 | 29.4 | 29.9 | 29.0 | 29.2 | 28.8 | 29.9 | 28.9 |
| F11 | 50.7 | 50.1 | 51.4 | 49.6 | 49.7 | 48.7 | 50.2 | 50.2 | 50.8 | 50.8 | 51.1 |
| F12 | 77.2 | 74.9 | 78.3 | 76.7 | 76.3 | 74.5 | 75.6 | 75.3 | 75.4 | 75.0 | 78.5 |
| F13 | 45.9 | 45.4 | 47.6 | 46.7 | 46.2 | 47.1 | 45.3 | 46.0 | 46.0 | 45.7 | 46.5 |
| F14 | 48.9 | 47.2 | 50.3 | 48.2 | 48.2 | 47.6 | 47.5 | 47.3 | 48.1 | 47.7 | 47.7 |
| F15 | 48.3 | 48.4 | 30.2 | 48.4 | 49.3 | 48.5 | 48.6 | 46.7 | 47.1 | 51.1 | 47.9 |
| F16 | 97.0 | 95.8 | 96.9 | 99.6 | 100.2 | 96.5 | 97.5 | 96.3 | 93.6 | 99.0 | 98.6 |
| F17 | 98.9 | 102.7 | 101.0 | 99.4 | 101.3 | 98.0 | 98.6 | 99.7 | 98.6 | 99.6 | 98.2 |
| F18 | 57.8 | 48.76 | 49.5 | 50.8 | 48.5 | 41.2 | 48.9 | 54.0 | 48.7 | 48.4 | 49.0 |

Both the pH values (Table 29) and protein concentrations (Table 30) appear to be quite stable throughout the course of Study 1.3. The monomer content by SEC ranged from about 98% to 98.5% at t0, with only dimer (RRT 0.89) and oligomer (RRT 0.85) being observed (Table 31). At no times were fragments seen by SEC in these studies.

TABLE 31
Percentage of peak areas by SEC for formulations in
Study 1.3 at t0 LB141 S1.3 t0 SEC

| | HMW3 | | | HMW2 | | | HMW1 | | | monomer | | |
|---|---|---|---|---|---|---|---|---|---|---|---|---|
| Sample | RRT 0.79 | | | RRT 0.85 | | | RRT 0.89 | | | RRT 1.00 | | |
| F01 | n/a | ± | n/a | 0.10 | ± | 0.00 | 1.38 | ± | 0.01 | 98.52 | ± | 0.01 |
| F02 | n/a | ± | n/a | 0.10 | ± | 0.00 | 1.36 | ± | 0.00 | 98.54 | ± | 0.00 |
| F03 | n/a | ± | n/a | 0.08 | ± | 0.00 | 1.47 | ± | 0.02 | 98.45 | ± | 0.02 |
| F04 | n/a | ± | n/a | 0.08 | ± | 0.00 | 1.54 | ± | 0.01 | 98.38 | ± | 0.01 |
| F05 | n/a | ± | n/a | 0.08 | ± | 0.00 | 1.42 | ± | 0.00 | 98.49 | ± | 0.00 |
| F06 | n/a | ± | n/a | 0.09 | ± | 0.00 | 1.35 | ± | 0.00 | 98.56 | ± | 0.00 |
| F07 | n/a | ± | n/a | 0.09 | ± | 0.00 | 1.48 | ± | 0.00 | 98.44 | ± | 0.01 |
| F08 | n/a | ± | n/a | 0.09 | ± | 0.00 | 1.58 | ± | 0.00 | 98.33 | ± | 0.01 |
| F09 | n/a | ± | n/a | 0.09 | ± | 0.00 | 1.81 | ± | 0.01 | 98.10 | ± | 0.01 |
| F10 | n/a | ± | n/a | 0.10 | ± | 0.00 | 1.50 | ± | 0.00 | 98.40 | ± | 0.00 |
| F11 | n/a | ± | n/a | 0.11 | ± | 0.00 | 1.47 | ± | 0.01 | 98.42 | ± | 0.01 |
| F12 | n/a | ± | n/a | 0.10 | ± | 0.00 | 1.66 | ± | 0.01 | 98.25 | ± | 0.01 |
| F13 | n/a | ± | n/a | 0.11 | ± | 0.00 | 1.53 | ± | 0.00 | 98.36 | ± | 0.00 |
| F14 | n/a | ± | n/a | 0.10 | ± | 0.00 | 1.55 | ± | 0.01 | 98.35 | ± | 0.01 |
| F15 | n/a | ± | n/a | 0.09 | ± | 0.00 | 1.67 | ± | 0.00 | 98.24 | ± | 0.01 |
| F16 | n/a | ± | n/a | 0.11 | ± | 0.00 | 1.73 | ± | 0.01 | 98.16 | ± | 0.01 |
| F17 | n/a | ± | n/a | 0.10 | ± | 0.00 | 1.89 | ± | 0.01 | 98.01 | ± | 0.01 |
| F18 | n/a | ± | n/a | 0.12 | ± | 0.00 | 1.72 | ± | 0.00 | 98.16 | ± | 0.00 |

TABLE 32
Percentage of peak areas by SEC for formulations
in Study 1.3 stored at 30° C. for one week
LB141 S1.3 t1 30 C. SEC

| | HMW3 | | | HMW2 | | | HMW1 | | | monomer | | |
|---|---|---|---|---|---|---|---|---|---|---|---|---|
| Sample | RRT 0.79 | | | RRT 0.85 | | | RRT 0.89 | | | RRT 1.00 | | |
| F01 | n/a | ± | n/a | 0.22 | ± | 0.00 | 1.81 | ± | 0.00 | 97.98 | ± | 0.00 |
| F02 | n/a | ± | n/a | 0.21 | ± | 0.00 | 1.73 | ± | 0.00 | 98.06 | ± | 0.00 |
| F03 | n/a | ± | n/a | 0.15 | ± | 0.00 | 1.81 | ± | 0.01 | 98.04 | ± | 0.00 |
| F04 | n/a | ± | n/a | 0.15 | ± | 0.00 | 1.95 | ± | 0.00 | 97.90 | ± | 0.01 |
| F05 | n/a | ± | n/a | 0.17 | ± | 0.00 | 1.77 | ± | 0.00 | 98.06 | ± | 0.00 |
| F06 | n/a | ± | n/a | 0.17 | ± | 0.00 | 1.76 | ± | 0.00 | 98.07 | ± | 0.00 |
| F07 | n/a | ± | n/a | 0.16 | ± | 0.00 | 1.85 | ± | 0.01 | 97.99 | ± | 0.01 |
| F08 | n/a | ± | n/a | 0.16 | ± | 0.00 | 1.98 | ± | 0.00 | 97.86 | ± | 0.00 |
| F09 | n/a | ± | n/a | 0.17 | ± | 0.12 | 2.24 | ± | 0.12 | 97.58 | ± | 0.01 |
| F10 | n/a | ± | n/a | 0.16 | ± | 0.00 | 1.92 | ± | 0.01 | 97.92 | ± | 0.01 |
| F11 | n/a | ± | n/a | 0.18 | ± | 0.00 | 1.89 | ± | 0.00 | 97.93 | ± | 0.00 |
| F12 | n/a | ± | n/a | 0.18 | ± | 0.00 | 1.92 | ± | 0.00 | 97.93 | ± | 0.00 |
| F14 | n/a | ± | n/a | 0.19 | ± | 0.00 | 2.00 | ± | 0.01 | 97.81 | ± | 0.00 |
| F15 | n/a | ± | n/a | 0.18 | ± | 0.00 | 1.96 | ± | 0.01 | 97.86 | ± | 0.00 |
| F16 | n/a | ± | n/a | 0.09 | ± | 0.11 | 2.31 | ± | 0.11 | 97.60 | ± | 0.01 |
| F17 | n/a | ± | n/a | 0.02 | ± | 0.00 | 2.42 | ± | 0.01 | 97.55 | ± | 0.01 |
| F18 | n/a | ± | n/a | 0.02 | ± | 0.00 | 2.36 | ± | 0.01 | 97.61 | ± | 0.01 |

At 30° C., no higher MW species are observed and the loss of monomer is slight (Table 32). The lowest monomer contents were seen for the 100 mg/mL formulations (formulations F16 and F17). On the other hand, at 40° C., higher MW species are observed, even after only one week (Table 33). While the differences from t0 are small, it appears that higher trehalose levels and lower arginine concentrations may be beneficial for stability. Little further decrease in monomer content is seen at two weeks at 30° C. over the one-week samples (Table 34). Again, no higher MW species (RRT 0.79) is observed. Similarly, there is little loss even after storage at 40° C. for two weeks (Table 35), with monomer contents still at 97% or higher for many formulations. The largest decreases are seen for formulations with no PS 80 or at high protein concentrations.

TABLE 33
Percentage of peak areas by SEC for formulations
in Study 1.3 stored at 40° C. for one week
LB141 S1.3 t1 40 C. SEC

| | HMW3 | | | HMW2 | | | HMW1 | | | monomer | | |
|---|---|---|---|---|---|---|---|---|---|---|---|---|
| Sample | RRT 0.79 | | | RRT 0.85 | | | RRT 0.89 | | | RRT 1.00 | | |
| F01 | 0.24 | ± | 0.00 | 0.21 | ± | 0.01 | 2.12 | ± | 0.03 | 97.42 | ± | 0.03 |
| F02 | 0.26 | ± | 0.00 | 0.24 | ± | 0.00 | 2.11 | ± | 0.01 | 97.39 | ± | 0.01 |
| F03 | 0.19 | ± | 0.00 | 0.19 | ± | 0.00 | 1.95 | ± | 0.02 | 97.82 | ± | 0.02 |
| F04 | 0.21 | ± | 0.01 | 0.21 | ± | 0.01 | 2.16 | ± | 0.01 | 97.57 | ± | 0.01 |
| F05 | 0.21 | ± | 0.01 | 0.21 | ± | 0.01 | 1.94 | ± | 0.00 | 97.80 | ± | 0.00 |
| F06 | 0.19 | ± | 0.00 | 0.19 | ± | 0.00 | 1.84 | ± | 0.01 | 97.92 | ± | 0.01 |
| F07 | 0.18 | ± | 0.00 | 0.18 | ± | 0.00 | 1.92 | ± | 0.01 | 97.87 | ± | 0.01 |
| F08 | 0.23 | ± | 0.00 | 0.23 | ± | 0.00 | 2.16 | ± | 0.01 | 97.55 | ± | 0.01 |
| F09 | 0.14 | ± | 0.11 | 0.26 | ± | 0.01 | 2.35 | ± | 0.02 | 97.31 | ± | 0.01 |
| F10 | 0.17 | ± | 0.01 | 0.17 | ± | 0.01 | 1.97 | ± | 0.01 | 97.82 | ± | 0.01 |
| F11 | 0.16 | ± | 0.10 | 0.22 | ± | 0.01 | 1.99 | ± | 0.01 | 97.75 | ± | 0.01 |
| F12 | 0.23 | ± | 0.01 | 0.23 | ± | 0.01 | 2.08 | ± | 0.02 | 97.63 | ± | 0.01 |
| F13 | 0.18 | ± | 0.01 | 0.18 | ± | 0.01 | 1.93 | ± | 0.01 | 97.85 | ± | 0.01 |
| F14 | 0.22 | ± | 0.01 | 0.22 | ± | 0.01 | 2.13 | ± | 0.01 | 97.57 | ± | 0.00 |
| F15 | 0.22 | ± | 0.01 | 0.22 | ± | 0.01 | 2.17 | ± | 0.01 | 97.50 | ± | 0.01 |
| F16 | 0.24 | ± | 0.01 | 0.28 | ± | 0.01 | 2.41 | ± | 0.00 | 97.07 | ± | 0.00 |
| F17 | 0.29 | ± | 0.01 | 0.29 | ± | 0.01 | 2.66 | ± | 0.02 | 96.89 | ± | 0.01 |
| F18 | 0.16 | ± | 0.03 | 0.16 | ± | 0.03 | 2.20 | ± | 0.03 | 97.50 | ± | 0.01 |

TABLE 34
Percentage of peak areas by SEC for formulations
in Study 1.3 stored at 30° C. for two weeks
LB141 S1.3 t2 30 C. SEC

| | HMW3 | | | HMW2 | | | HMW1 | | | monomer | | |
|---|---|---|---|---|---|---|---|---|---|---|---|---|
| Sample | RRT 0.79 | | | RRT 0.85 | | | RRT 0.89 | | | RRT 1.00 | | |
| F01 | n/a | ± | n/a | 0.24 | ± | 0.01 | 1.85 | ± | 0.01 | 97.91 | ± | 0.01 |
| F02 | n/a | ± | n/a | 0.24 | ± | 0.01 | 1.83 | ± | 0.01 | 97.93 | ± | 0.01 |
| F03 | n/a | ± | n/a | 0.18 | ± | 0.00 | 1.82 | ± | 0.00 | 98.00 | ± | 0.00 |
| F04 | n/a | ± | n/a | 0.19 | ± | 0.00 | 1.91 | ± | 0.00 | 97.90 | ± | 0.00 |
| F05 | n/a | ± | n/a | 0.20 | ± | 0.00 | 1.83 | ± | 0.00 | 97.96 | ± | 0.01 |
| F06 | n/a | ± | n/a | 0.20 | ± | 0.01 | 1.80 | ± | 0.00 | 98.00 | ± | 0.01 |
| F07 | n/a | ± | n/a | 0.18 | ± | 0.00 | 1.91 | ± | 0.00 | 97.91 | ± | 0.00 |
| F08 | n/a | ± | n/a | 0.20 | ± | 0.00 | 1.97 | ± | 0.00 | 97.83 | ± | 0.00 |
| F09 | n/a | ± | n/a | 0.17 | ± | 0.01 | 2.03 | ± | 0.01 | 97.80 | ± | 0.03 |
| F10 | n/a | ± | n/a | 0.16 | ± | 0.01 | 1.88 | ± | 0.01 | 97.96 | ± | 0.01 |
| F11 | n/a | ± | n/a | 0.19 | ± | 0.00 | 1.89 | ± | 0.02 | 97.92 | ± | 0.02 |
| F12 | n/a | ± | n/a | 0.18 | ± | 0.01 | 1.88 | ± | 0.02 | 97.94 | ± | 0.02 |
| F13 | n/a | ± | n/a | 0.18 | ± | 0.01 | 1.83 | ± | 0.01 | 98.00 | ± | 0.02 |
| F14 | n/a | ± | n/a | 0.17 | ± | 0.00 | 1.93 | ± | 0.01 | 97.90 | ± | 0.00 |
| F15 | n/a | ± | n/a | 0.16 | ± | 0.01 | 1.78 | ± | 0.02 | 98.05 | ± | 0.02 |
| F16 | n/a | ± | n/a | 0.19 | ± | 0.01 | 2.05 | ± | 0.01 | 97.76 | ± | 0.02 |
| F17 | n/a | ± | n/a | 0.18 | ± | 0.00 | 2.14 | ± | 0.01 | 97.68 | ± | 0.01 |
| F18 | n/a | ± | n/a | 0.17 | ± | 0.00 | 2.04 | ± | 0.01 | 97.79 | ± | 0.02 |

TABLE 35
Percentage of peak areas by SEC for formulations
in Study 1.3 stored at 40° C. for two weeks
LB141 S1.3 t2 40 C. SEC

| | HMW3 | | | HMW2 | | | HMW1 | | | monomer | | |
|---|---|---|---|---|---|---|---|---|---|---|---|---|
| Sample | RRT 0.79 | | | RRT 0.85 | | | RRT 0.89 | | | RRT 1.00 | | |
| F01 | 0.61 | ± | 0.01 | 0.24 | ± | 0.01 | 2.24 | ± | 0.01 | 96.92 | ± | 0.01 |
| F02 | 0.64 | ± | 0.01 | 0.21 | ± | 0.02 | 2.16 | ± | 0.02 | 96.98 | ± | 0.00 |
| F03 | 0.10 | ± | 0.01 | 0.21 | ± | 0.02 | 2.19 | ± | 0.03 | 97.50 | ± | 0.05 |
| F04 | 0.13 | ± | 0.00 | 0.25 | ± | 0.01 | 2.23 | ± | 0.01 | 97.38 | ± | 0.01 |
| F05 | 0.09 | ± | 0.00 | 0.23 | ± | 0.01 | 2.05 | ± | 0.02 | 97.64 | ± | 0.02 |
| F06 | 0.06 | ± | 0.00 | 0.20 | ± | 0.01 | 1.97 | ± | 0.03 | 97.78 | ± | 0.01 |
| F07 | 0.04 | ± | 0.00 | 0.17 | ± | 0.00 | 1.94 | ± | 0.01 | 97.85 | ± | 0.01 |
| F08 | 0.34 | ± | 0.01 | 0.26 | ± | 0.01 | 2.14 | ± | 0.01 | 97.26 | ± | 0.03 |
| F09 | 0.65 | ± | 0.03 | 0.31 | ± | 0.00 | 2.33 | ± | 0.03 | 96.72 | ± | 0.05 |
| F10 | 0.15 | ± | 0.00 | 0.21 | ± | 0.01 | 2.01 | ± | 0.04 | 97.63 | ± | 0.04 |
| F11 | 0.42 | ± | 0.00 | 0.24 | ± | 0.01 | 2.10 | ± | 0.01 | 97.24 | ± | 0.02 |
| F12 | 0.35 | ± | 0.01 | 0.27 | ± | 0.01 | 2.16 | ± | 0.02 | 97.22 | ± | 0.02 |
| F13 | 0.08 | ± | 0.00 | 0.20 | ± | 0.01 | 2.35 | ± | 0.01 | 97.37 | ± | 0.01 |
| F14 | 0.17 | ± | 0.02 | 0.25 | ± | 0.00 | 2.45 | ± | 0.02 | 97.13 | ± | 0.01 |
| F15 | 0.32 | ± | 0.01 | 0.35 | ± | 0.00 | 2.56 | ± | 0.02 | 96.77 | ± | 0.02 |
| F16 | 1.01 | ± | 0.03 | 0.49 | ± | 0.02 | 2.73 | ± | 0.04 | 95.77 | ± | 0.06 |
| F17 | 1.50 | ± | 0.03 | 0.46 | ± | 0.02 | 2.54 | ± | 0.01 | 95.50 | ± | 0.02 |
| F18 | 0.89 | ± | 0.00 | 0.45 | ± | 0.01 | 2.76 | ± | 0.01 | 95.90 | ± | 0.01 |

TABLE 36
Percentage of peak areas by SEC for formulations
in Study 1.3 stored at 30° C. for three weeks
LB141 S1.3 t3 30 C. SEC

| | HMW3 | | | HMW2 | | | HMW1 | | | monomer | | |
|---|---|---|---|---|---|---|---|---|---|---|---|---|
| Sample | RRT 0.79 | | | RRT 0.85 | | | RRT 0.89 | | | RRT 1.00 | | |
| F01 | n/a | ± | n/a | 0.14 | ± | 0.02 | 1.85 | ± | 0.01 | 98.06 | ± | 0.01 |
| F02 | n/a | ± | n/a | 0.13 | ± | 0.01 | 1.83 | ± | 0.01 | 98.11 | ± | 0.01 |
| F03 | n/a | ± | n/a | 0.10 | ± | 0.03 | 1.82 | ± | 0.00 | 98.21 | ± | 0.01 |
| F04 | n/a | ± | n/a | 0.11 | ± | 0.02 | 1.91 | ± | 0.00 | 98.10 | ± | 0.01 |
| F05 | n/a | ± | n/a | 0.10 | ± | 0.00 | 1.83 | ± | 0.00 | 98.12 | ± | 0.01 |
| F06 | n/a | ± | n/a | 0.11 | ± | 0.03 | 1.80 | ± | 0.00 | 98.13 | ± | 0.01 |
| F07 | n/a | ± | n/a | 0.11 | ± | 0.00 | 1.91 | ± | 0.00 | 98.05 | ± | 0.04 |
| F08 | n/a | ± | n/a | 0.11 | ± | 0.01 | 1.97 | ± | 0.00 | 97.97 | ± | 0.01 |
| F09 | n/a | ± | n/a | 0.15 | ± | 0.02 | 2.03 | ± | 0.01 | 97.82 | ± | 0.02 |
| F10 | n/a | ± | n/a | 0.10 | ± | 0.01 | 1.88 | ± | 0.01 | 98.03 | ± | 0.01 |
| F11 | n/a | ± | n/a | 0.13 | ± | 0.00 | 1.89 | ± | 0.02 | 97.95 | ± | 0.00 |
| F12 | n/a | ± | n/a | 0.12 | ± | 0.01 | 1.88 | ± | 0.02 | 97.95 | ± | 0.03 |
| F13 | n/a | ± | n/a | 0.14 | ± | 0.03 | 1.83 | ± | 0.01 | 98.00 | ± | 0.00 |
| F14 | n/a | ± | n/a | 0.15 | ± | 0.05 | 1.93 | ± | 0.01 | 97.85 | ± | 0.01 |
| F15 | n/a | ± | n/a | 0.12 | ± | 0.01 | 1.78 | ± | 0.02 | 97.99 | ± | 0.02 |
| F16 | n/a | ± | n/a | 0.13 | ± | 0.02 | 2.05 | ± | 0.01 | 97.81 | ± | 0.03 |
| F17 | n/a | ± | n/a | 0.12 | ± | 0.02 | 2.14 | ± | 0.01 | 97.72 | ± | 0.02 |
| F18 | n/a | ± | n/a | 0.12 | ± | 0.01 | 2.04 | ± | 0.01 | 97.79 | ± | 0.02 |

Even after three weeks at 30° C., there is little loss in monomer content for most of the formulations (Table 36), suggesting that these are nearly optimal in terms of stability. For the samples held at 25° C. for four weeks, the monomer contents still hover near 98%, which is near the levels at t0 (Table 37). These short-term studies all indicate that the formulations being considered here are nearly optimal. In other words, the physical stability measured by SEC is greatest near pH 6.0 in citrate buffer, where arginine and trehalose are acting in concert as stabilizers. It may be that PS 80 is not necessary, or even detrimental to long-term stability.

As with Study 1.2, higher protein concentrations appear to reduce the storage stability of albiglutide.

TABLE 37
Percentage of peak areas by SEC for formulations in Study 1.3 stored at 25°
C. for four weeks
LB141 S1.3 t4 25 C. SEC

| | HMW3 | | | HMW2 | | | HMW1 | | | monomer | | |
|---|---|---|---|---|---|---|---|---|---|---|---|---|
| Sample | RRT 0.79 | | | RRT 0.85 | | | RRT 0.89 | | | RRT 1.00 | | |
| F01 | n/a | ± | n/a | 0.19 | ± | 0.00 | 1.68 | ± | 0.00 | 98.12 | ± | 0.00 |
| F02 | n/a | ± | n/a | 0.18 | ± | 0.01 | 1.63 | ± | 0.01 | 98.18 | ± | 0.02 |
| F03 | n/a | ± | n/a | 0.12 | ± | 0.01 | 1.72 | ± | 0.01 | 98.16 | ± | 0.02 |
| F04 | n/a | ± | n/a | 0.14 | ± | 0.00 | 1.84 | ± | 0.00 | 98.03 | ± | 0.01 |
| F05 | n/a | ± | n/a | 0.17 | ± | 0.00 | 1.75 | ± | 0.01 | 98.09 | ± | 0.00 |
| F06 | n/a | ± | n/a | 0.17 | ± | 0.00 | 1.72 | ± | 0.00 | 98.12 | ± | 0.01 |
| F07 | n/a | ± | n/a | 0.14 | ± | 0.00 | 1.84 | ± | 0.01 | 98.02 | ± | 0.01 |
| F08 | n/a | ± | n/a | 0.15 | ± | 0.01 | 1.91 | ± | 0.01 | 97.94 | ± | 0.02 |
| F09 | n/a | ± | n/a | 0.19 | ± | 0.01 | 2.02 | ± | 0.01 | 97.78 | ± | 0.01 |
| F10 | n/a | ± | n/a | 0.15 | ± | 0.01 | 1.88 | ± | 0.01 | 97.97 | ± | 0.01 |
| F11 | n/a | ± | n/a | 0.18 | ± | 0.01 | 1.87 | ± | 0.00 | 97.95 | ± | 0.00 |
| F12 | n/a | ± | n/a | 0.18 | ± | 0.01 | 1.86 | ± | 0.02 | 97.96 | ± | 0.01 |
| F13 | n/a | ± | n/a | 0.17 | ± | 0.00 | 1.80 | ± | 0.00 | 98.02 | ± | 0.00 |
| F14 | n/a | ± | n/a | 0.16 | ± | 0.01 | 1.87 | ± | 0.00 | 97.96 | ± | 0.02 |
| F15 | n/a | ± | n/a | 0.15 | ± | 0.00 | 1.78 | ± | 0.01 | 98.07 | ± | 0.01 |
| F16 | n/a | ± | n/a | 0.18 | ± | 0.01 | 1.97 | ± | 0.01 | 97.84 | ± | 0.02 |
| F17 | n/a | ± | n/a | 0.21 | ± | 0.00 | 2.01 | ± | 0.01 | 97.78 | ± | 0.01 |
| F18 | n/a | ± | n/a | 0.17 | ± | 0.01 | 1.96 | ± | 0.01 | 97.88 | ± | 0.00 |

TABLE 38
Percentage of peak areas by SEC for formulations in Study 1.3 stored at 5° C.
for thirteen weeks (three months)
LB141 S1.3 t13 5 C. SEC

| | HMW3 | | | HMW2 | | | HMW1 | | | monomer | | |
|---|---|---|---|---|---|---|---|---|---|---|---|---|
| Sample | RRT 0.79 | | | RRT 0.85 | | | RRT 0.89 | | | RRT 1.00 | | |
| F01 | n/a | ± | n/a | 0.22 | ± | 0.01 | 1.85 | ± | 0.01 | 97.93 | ± | 0.01 |
| F02 | n/a | ± | n/a | 0.21 | ± | 0.00 | 1.75 | ± | 0.00 | 98.04 | ± | 0.00 |
| F03 | n/a | ± | n/a | 0.16 | ± | 0.01 | 1.83 | ± | 0.01 | 98.01 | ± | 0.00 |
| F04 | n/a | ± | n/a | 0.15 | ± | 0.01 | 1.92 | ± | 0.01 | 97.92 | ± | 0.00 |
| F05 | n/a | ± | n/a | 0.17 | ± | 0.01 | 1.81 | ± | 0.01 | 98.02 | ± | 0.01 |
| F06 | n/a | ± | n/a | 0.17 | ± | 0.00 | 1.76 | ± | 0.00 | 98.07 | ± | 0.00 |
| F07 | n/a | ± | n/a | 0.16 | ± | 0.00 | 1.82 | ± | 0.00 | 98.02 | ± | 0.01 |
| F08 | n/a | ± | n/a | 0.16 | ± | 0.00 | 1.91 | ± | 0.01 | 97.94 | ± | 0.01 |
| F09 | n/a | ± | n/a | 0.16 | ± | 0.00 | 2.06 | ± | 0.01 | 97.78 | ± | 0.01 |
| F10 | n/a | ± | n/a | 0.16 | ± | 0.00 | 1.79 | ± | 0.00 | 98.05 | ± | 0.00 |
| F11 | n/a | ± | n/a | 0.17 | ± | 0.01 | 1.82 | ± | 0.01 | 98.01 | ± | 0.01 |
| F12 | n/a | ± | n/a | 0.16 | ± | 0.00 | 1.89 | ± | 0.01 | 97.95 | ± | 0.00 |
| F13 | n/a | ± | n/a | 0.17 | ± | 0.00 | 1.81 | ± | 0.00 | 98.02 | ± | 0.00 |
| F14 | n/a | ± | n/a | 0.17 | ± | 0.01 | 1.88 | ± | 0.01 | 97.95 | ± | 0.00 |
| F15 | n/a | ± | n/a | 0.15 | ± | 0.01 | 1.94 | ± | 0.01 | 97.91 | ± | 0.00 |
| F16 | n/a | ± | n/a | 0.16 | ± | 0.00 | 1.96 | ± | 0.01 | 97.87 | ± | 0.00 |
| F17 | n/a | ± | n/a | 0.15 | ± | 0.00 | 2.04 | ± | 0.00 | 97.80 | ± | 0.00 |
| F18 | n/a | ± | n/a | 0.18 | ± | 0.00 | 2.06 | ± | 0.01 | 97.76 | ± | 0.01 |

Longer term storage studies were conducted, holding samples at 5° C. and 25° C. for three and five months (t13 and t22, respectively). After three months at 5° C., the monomer content by Sec remained near 98% for many, if not all, of the formulations (Table 38). By comparison, those held at 25° C. for the same length of time contained about 97% monomer (Table 39), indicating that these formulations were all quite stable.

TABLE 39
Percentage of peak areas by SEC for formulations in Study 1.3 stored at 25° C.
for thirteen weeks (three months)
LB141 S1.3 t13 25 C. SEC

| | HMW3 | | | HMW2 | | | HMW1 | | | monomer | | |
|---|---|---|---|---|---|---|---|---|---|---|---|---|
| Sample | RRT 0.79 | | | RRT 0.85 | | | RRT 0.89 | | | RRT 1.00 | | |
| F01 | n/a | ± | n/a | 0.37 | ± | 0.01 | 2.55 | ± | 0.02 | 97.08 | ± | 0.01 |
| F02 | n/a | ± | n/a | 0.35 | ± | 0.02 | 2.46 | ± | 0.02 | 97.19 | ± | 0.00 |
| F03 | n/a | ± | n/a | 0.23 | ± | 0.01 | 2.44 | ± | 0.01 | 97.33 | ± | 0.00 |
| F04 | n/a | ± | n/a | 0.24 | ± | 0.01 | 2.58 | ± | 0.01 | 97.18 | ± | 0.00 |
| F05 | n/a | ± | n/a | 0.25 | ± | 0.01 | 2.46 | ± | 0.01 | 97.29 | ± | 0.00 |
| F06 | n/a | ± | n/a | 0.26 | ± | 0.01 | 2.44 | ± | 0.01 | 97.31 | ± | 0.00 |
| F07 | n/a | ± | n/a | 0.24 | ± | 0.01 | 2.56 | ± | 0.01 | 97.20 | ± | 0.00 |
| F08 | n/a | ± | n/a | 0.24 | ± | 0.01 | 2.63 | ± | 0.01 | 97.13 | ± | 0.00 |
| F09 | n/a | ± | n/a | 0.25 | ± | 0.04 | 2.76 | ± | 0.02 | 96.98 | ± | 0.04 |
| F10 | n/a | ± | n/a | 0.26 | ± | 0.00 | 2.51 | ± | 0.02 | 97.24 | ± | 0.02 |
| F11 | n/a | ± | n/a | 0.27 | ± | 0.00 | 2.59 | ± | 0.00 | 97.14 | ± | 0.00 |
| F12 | n/a | ± | n/a | 0.25 | ± | 0.01 | 2.57 | ± | 0.00 | 97.18 | ± | 0.00 |
| F13 | n/a | ± | n/a | 0.25 | ± | 0.00 | 2.45 | ± | 0.01 | 97.30 | ± | 0.01 |
| F14 | n/a | ± | n/a | 0.27 | ± | 0.01 | 2.63 | ± | 0.02 | 97.10 | ± | 0.01 |
| F15 | n/a | ± | n/a | 0.23 | ± | 0.00 | 2.57 | ± | 0.00 | 97.20 | ± | 0.00 |
| F16 | n/a | ± | n/a | 0.31 | ± | 0.00 | 2.80 | ± | 0.01 | 96.89 | ± | 0.00 |
| F17 | n/a | ± | n/a | 0.30 | ± | 0.00 | 2.85 | ± | 0.00 | 96.86 | ± | 0.00 |
| F18 | n/a | ± | n/a | 0.31 | ± | 0.01 | 2.74 | ± | 0.01 | 96.95 | ± | 0.01 |

TABLE 40
Percentage of peak areas by SEC for formulations in Study 1.3 stored at 5° C.
for 22 weeks (five months)
LB141 S1.3 5MO 5 C. SEC

| | HMW3 | | | HMW2 | | | HMW1 | | | monomer | | |
|---|---|---|---|---|---|---|---|---|---|---|---|---|
| Sample | RRT 0.79 | | | RRT 0.85 | | | RRT 0.89 | | | RRT 1.00 | | |
| F01 | n/a | ± | n/a | 0.22 | ± | 0.01 | 1.85 | ± | 0.00 | 97.93 | ± | 0.01 |
| F02 | n/a | ± | n/a | 0.22 | ± | 0.00 | 1.78 | ± | 0.01 | 98.00 | ± | 0.00 |
| F03 | n/a | ± | n/a | 0.17 | ± | 0.01 | 1.91 | ± | 0.01 | 97.92 | ± | 0.00 |
| F04 | n/a | ± | n/a | 0.18 | ± | 0.00 | 2.00 | ± | 0.01 | 97.82 | ± | 0.00 |
| F05 | n/a | ± | n/a | 0.19 | ± | 0.00 | 1.95 | ± | 0.01 | 97.86 | ± | 0.00 |
| F06 | n/a | ± | n/a | 0.18 | ± | 0.00 | 1.85 | ± | 0.01 | 97.96 | ± | 0.01 |
| F07 | n/a | ± | n/a | 0.17 | ± | 0.00 | 1.91 | ± | 0.01 | 97.92 | ± | 0.01 |
| F08 | n/a | ± | n/a | 0.18 | ± | 0.01 | 2.02 | ± | 0.00 | 97.80 | ± | 0.00 |
| F09 | n/a | ± | n/a | 0.21 | ± | 0.01 | 2.13 | ± | 0.01 | 97.67 | ± | 0.01 |
| F10 | n/a | ± | n/a | 0.17 | ± | 0.01 | 1.87 | ± | 0.01 | 97.97 | ± | 0.00 |
| F11 | n/a | ± | n/a | 0.19 | ± | 0.01 | 1.91 | ± | 0.01 | 97.90 | ± | 0.00 |
| F12 | n/a | ± | n/a | 0.20 | ± | 0.01 | 1.92 | ± | 0.01 | 97.88 | ± | 0.00 |
| F13 | n/a | ± | n/a | 0.18 | ± | 0.01 | 1.87 | ± | 0.01 | 97.95 | ± | 0.00 |
| F14 | n/a | ± | n/a | 0.19 | ± | 0.00 | 1.90 | ± | 0.01 | 97.91 | ± | 0.00 |
| F15 | n/a | ± | n/a | 0.18 | ± | 0.01 | 1.93 | ± | 0.01 | 97.89 | ± | 0.00 |
| F16 | n/a | ± | n/a | 0.21 | ± | 0.00 | 1.98 | ± | 0.00 | 97.81 | ± | 0.00 |
| F17 | n/a | ± | n/a | 0.21 | ± | 0.01 | 2.08 | ± | 0.01 | 97.71 | ± | 0.01 |
| F18 | n/a | ± | n/a | 0.20 | ± | 0.00 | 2.06 | ± | 0.00 | 97.74 | ± | 0.00 |

As one examines the samples held for five months at 5° C., the monomer content by SEC is just before 98% on average (Table 40). This amounts to about ˜0.1% loss (or less) of monomer for each month of storage. Such a degradation rate would predict a loss of 2 to 3% over the course of 24 months, with the monomer content being near 96% at the end of expiry. Meanwhile, storage of formulations at 25° C. for five months shows that most still retain ˜97% monomer (Table 41), suggesting that excursions to room temperature for weeks could be accommodated.

TABLE 41
Percentage of peak areas by SEC for formulations in Study 1.3 stored at 25° C.
for 22 weeks (five months)
LB141 S1.3 5MO 25 C. SEC

| | HMW3 | | | HMW2 | | | HMW1 | | | monomer | | |
|---|---|---|---|---|---|---|---|---|---|---|---|---|
| Sample | RRT 0.79 | | | RRT 0.85 | | | RRT 0.89 | | | RRT 1.00 | | |
| F01 | n/a | ± | n/a | 0.31 | ± | 0.00 | 2.77 | ± | 0.01 | 96.92 | ± | 0.01 |
| F02 | n/a | ± | n/a | 0.30 | ± | 0.01 | 2.69 | ± | 0.00 | 97.01 | ± | 0.01 |
| F03 | n/a | ± | n/a | 0.20 | ± | 0.00 | 2.62 | ± | 0.00 | 97.18 | ± | 0.00 |
| F04 | n/a | ± | n/a | 0.22 | ± | 0.00 | 2.85 | ± | 0.01 | 96.94 | ± | 0.01 |
| F05 | n/a | ± | n/a | 0.23 | ± | 0.01 | 2.69 | ± | 0.01 | 97.08 | ± | 0.01 |
| F06 | n/a | ± | n/a | 0.23 | ± | 0.00 | 2.62 | ± | 0.01 | 97.15 | ± | 0.01 |
| F07 | n/a | ± | n/a | 0.20 | ± | 0.01 | 2.83 | ± | 0.00 | 96.97 | ± | 0.00 |
| F08 | n/a | ± | n/a | 0.22 | ± | 0.00 | 2.82 | ± | 0.01 | 96.95 | ± | 0.00 |
| F09 | n/a | ± | n/a | 0.26 | ± | 0.01 | 2.99 | ± | 0.01 | 96.76 | ± | 0.00 |
| F10 | n/a | ± | n/a | 0.19 | ± | 0.00 | 2.70 | ± | 0.01 | 97.11 | ± | 0.01 |
| F11 | n/a | ± | n/a | 0.24 | ± | 0.01 | 2.78 | ± | 0.01 | 96.98 | ± | 0.01 |
| F12 | n/a | ± | n/a | 0.25 | ± | 0.01 | 2.80 | ± | 0.01 | 96.95 | ± | 0.01 |
| F13 | n/a | ± | n/a | 0.23 | ± | 0.01 | 2.64 | ± | 0.00 | 97.14 | ± | 0.00 |
| F14 | n/a | ± | n/a | 0.24 | ± | 0.00 | 2.81 | ± | 0.01 | 96.96 | ± | 0.00 |
| F15 | n/a | ± | n/a | 0.22 | ± | 0.00 | 2.68 | ± | 0.00 | 97.10 | ± | 0.00 |
| F16 | n/a | ± | n/a | 0.28 | ± | 0.01 | 2.93 | ± | 0.01 | 96.79 | ± | 0.00 |
| F17 | n/a | ± | n/a | 0.28 | ± | 0.01 | 3.05 | ± | 0.01 | 96.67 | ± | 0.00 |
| F18 | n/a | ± | n/a | 0.27 | ± | 0.01 | 2.92 | ± | 0.01 | 96.81 | ± | 0.00 |

Stability was also monitored by measuring the purity by RP HPLC, which provides some evidence of the chemical stability of these albiglutide formulations. The initial purity was near 89% for all of the formulations (Table 42). As samples are held at 30° C. (Table 43) or 25° C. (Table 44), the purities change very little, if at all (see below). At 40° C., there is some decrease in purity that appears to be real, but the purity is reduced only to levels near 86% (Table 42). The lowest purities are seen for samples with protein concentrations of 80 to 100 mg/mL. It should be noted that the 100 mg/mL containing PS 80 (F17) does display greater stability by RP HPLC at 40° C. than the corresponding formulation (F16) without the surfactant. These differences become small or insignificant at lower storage temperatures.

TABLE 42
Purity as measured by RP HPLC for the formulations in
Study 1.3 stored at 40° C. for zero, one, and two weeks

| Sample | t0 | t1 | t2 |
|---|---|---|---|
| F01 | 88.15 ± 0.50 | 88.90 ± 0.41 | 87.04 ± 1.07 |
| F02 | 88.64 ± 0.18 | 88.85 ± 0.75 | 87.12 ± 0.36 |
| F03 | 88.43 ± 0.45 | 88.55 ± 0.70 | 88.30 ± 0.06 |
| F04 | 86.94 ± 0.10 | 88.42 ± 0.28 | 86.64 ± 0.35 |
| F05 | 88.56 ± 0.20 | 89.57 ± 0.21 | 88.54 ± 0.12 |
| F06 | 87.67 ± 0.41 | 89.70 ± 0.55 | 87.73 ± 0.04 |
| F07 | 88.55 ± 0.30 | 88.71 ± 0.10 | 88.23 ± 0.48 |
| F08 | 87.90 ± 0.35 | 87.85 ± 0.21 | 87.57 ± 0.55 |
| F09 | 84.97 ± 0.32 | 88.09 ± 1.06 | 86.15 ± 0.20 |
| F10 | 88.76 ± 0.04 | 88.67 ± 0.53 | 87.93 ± 0.25 |
| F11 | 88.76 ± 0.21 | 89.53 ± 0.23 | 87.22 ± 0.33 |
| F12 | 86.25 ± 0.54 | 89.60 ± 0.28 | 86.69 ± 0.38 |
| F13 | 88.02 ± 0.27 | 90.04 ± 0.25 | 87.69 ± 0.22 |
| F14 | 87.42 ± 0.36 | 89.70 ± 0.32 | 88.10 ± 0.21 |
| F15 | 86.45 ± 0.06 | 89.44 ± 0.21 | 87.20 ± 0.51 |
| F16 | 86.48 ± 0.18 | 89.82 ± 0.45 | 86.45 ± 0.41 |
| F17 | 87.29 ± 0.24 | 89.19 ± 0.32 | 89.46 ± 0.38 |
| F18 | 87.75 ± 0.30 | 90.47 ± 0.06 | 86.84 ± 0.24 |

TABLE 43
Purity as measured by RP HPLC for the formulations
in Study 1.3 stored at 30° C. for zero, one, two and three weeks

| Sample | t0 | t1 30 | t2 30 | t3 30 |
|---|---|---|---|---|
| F01 | 88.15 ± 0.50 | 89.46 ± 0.10 | 89.24 ± 0.16 | 90.47 ± 0.37 |
| F02 | 88.64 ± 0.18 | 89.65 ± 0.43 | 89.51 ± 0.27 | 90.08 ± 0.41 |
| F03 | 88.43 ± 0.45 | 89.67 ± 0.29 | 89.07 ± 0.16 | 90.66 ± 0.27 |
| F04 | 86.94 ± 0.10 | 88.09 ± 0.63 | 88.27 ± 0.07 | 90.40 ± 0.60 |
| F05 | 88.56 ± 0.20 | 89.39 ± 0.14 | 89.36 ± 0.40 | 89.40 ± 0.19 |
| F06 | 87.67 ± 0.41 | 90.07 ± 0.10 | 88.70 ± 0.30 | 89.18 ± 0.09 |
| F07 | 88.55 ± 0.30 | 89.96 ± 0.30 | 88.49 ± 0.34 | 89.05 ± 0.19 |
| F08 | 87.90 ± 0.35 | 89.14 ± 0.65 | 87.69 ± 0.37 | 88.75 ± 0.31 |
| F09 | 84.97 ± 0.32 | 88.77 ± 0.23 | 88.18 ± 0.31 | 88.38 ± 0.66 |
| F10 | 88.76 ± 0.04 | 89.35 ± 0.34 | 89.37 ± 0.71 | 88.46 ± 0.46 |
| F11 | 88.76 ± 0.21 | 88.97 ± 0.15 | 89.17 ± 0.17 | 89.11 ± 0.49 |
| F12 | 86.25 ± 0.54 | 88.96 ± 0.54 | 88.83 ± 0.32 | 89.21 ± 0.40 |
| F13 | 88.02 ± 0.27 | 88.80 ± 0.44 | 88.90 ± 0.34 | 89.68 ± 0.55 |
| F14 | 87.42 ± 0.36 | 89.32 ± 0.32 | 89.35 ± 0.13 | 89.32 ± 0.70 |
| F15 | 86.45 ± 0.06 | 89.30 ± 0.32 | 89.68 ± 0.34 | 89.11 ± 0.40 |
| F16 | 86.48 ± 0.18 | 88.12 ± 0.55 | 86.78 ± 0.14 | 88.50 ± 0.80 |
| F17 | 87.29 ± 0.24 | 88.40 ± 0.68 | 87.50 ± 0.41 | 88.42 ± 0.64 |
| F18 | 87.75 ± 0.30 | 89.45 ± 0.28 | 88.72 ± 0.02 | 87.74 ± 0.41 |

TABLE 44
Purity as measured by RP HPLC for the formulations
in Study 1.3 stored at 25° C.

| Sample | t0 | t4 | t13 | t22 |
|---|---|---|---|---|
| F01 | 88.15 ± 0.50 | 88.08 ± 0.30 | 85.83 ± 1.41 | 87.96 ± 0.44 |
| F02 | 88.64 ± 0.18 | 87.07 ± 0.77 | 86.16 ± 0.55 | 87.78 ± 0.51 |
| F03 | 88.43 ± 0.45 | 86.57 ± 0.69 | 85.43 ± 0.91 | 86.80 ± 1.61 |
| F04 | 86.94 ± 0.10 | 86.50 ± 0.34 | 86.63 ± 0.86 | 87.08 ± 0.83 |
| F05 | 88.56 ± 0.20 | 86.34 ± 0.60 | 86.45 ± 1.06 | 86.78 ± 0.63 |
| F06 | 87.67 ± 0.41 | 86.41 ± 0.75 | 85.66 ± 0.88 | 87.74 ± 1.06 |
| F07 | 88.55 ± 0.30 | 86.90 ± 0.45 | 86.10 ± 0.58 | 85.94 ± 0.17 |
| F08 | 87.90 ± 0.35 | 86.79 ± 0.17 | 86.43 ± 0.11 | 85.17 ± 0.22 |
| F09 | 84.97 ± 0.32 | 87.06 ± 0.49 | 84.61 ± 0.42 | 84.64 ± 0.42 |
| F10 | 88.76 ± 0.04 | 87.33 ± 0.51 | 85.60 ± 0.49 | 85.27 ± 0.39 |
| F11 | 88.76 ± 0.21 | 86.88 ± 0.41 | 86.76 ± 0.51 | 84.95 ± 0.12 |
| F12 | 86.25 ± 0.54 | 86.76 ± 0.46 | 85.25 ± 0.38 | 84.40 ± 0.40 |
| F13 | 88.02 ± 0.27 | 87.35 ± 0.35 | 84.89 ± 0.97 | 83.95 ± 0.50 |
| F14 | 87.42 ± 0.36 | 87.62 ± 1.32 | 84.33 ± 0.91 | 83.98 ± 0.42 |
| F15 | 86.45 ± 0.06 | 87.11 ± 0.08 | 88.02 ± 0.15 | 84.18 ± 0.65 |
| F16 | 86.48 ± 0.18 | 87.60 ± 0.25 | 87.63 ± 0.44 | 83.96 ± 0.37 |
| F17 | 87.29 ± 0.24 | 88.09 ± 0.19 | 87.18 ± 1.30 | 84.14 ± 0.78 |
| F18 | 87.75 ± 0.30 | 88.19 ± 0.64 | 87.84 ± 0.48 | 85.27 ± 0.45 |

TABLE 45
Purity as measured by RP HPLC for the formulations
in Study 1.3 stored at 5° C.

| Sample | t0 | t13 | t22 |
|---|---|---|---|
| F01 | 88.15 ± 0.50 | 88.94 ± 0.56 | 88.72 ± 0.27 |
| F02 | 88.64 ± 0.18 | 87.30 ± 0.49 | 88.89 ± 0.57 |
| F03 | 88.43 ± 0.45 | 89.43 ± 0.58 | 89.44 ± 0.38 |
| F04 | 86.94 ± 0.10 | 85.64 ± 0.64 | 89.51 ± 0.10 |
| F05 | 88.56 ± 0.20 | 86.82 ± 0.34 | 89.03 ± 0.42 |
| F06 | 87.67 ± 0.41 | 87.54 ± 0.77 | 89.36 ± 0.26 |
| F07 | 88.55 ± 0.30 | 85.98 ± 0.68 | 89.26 ± 0.21 |
| F08 | 87.90 ± 0.35 | 88.40 ± 0.74 | 88.20 ± 0.77 |
| F09 | 84.97 ± 0.32 | 86.69 ± 0.25 | 88.43 ± 0.73 |
| F10 | 88.76 ± 0.04 | 88.29 ± 0.63 | 89.83 ± 0.37 |
| F11 | 88.76 ± 0.21 | 86.87 ± 0.61 | 89.68 ± 0.41 |
| F12 | 86.25 ± 0.54 | 87.52 ± 0.34 | 89.29 ± 0.28 |
| F13 | 88.02 ± 0.27 | 88.70 ± 0.10 | 89.99 ± 0.30 |
| F14 | 87.42 ± 0.36 | 87.63 ± 0.64 | 90.22 ± 0.51 |
| F15 | 86.45 ± 0.06 | 86.60 ± 0.28 | 90.37 ± 0.40 |
| F16 | 86.48 ± 0.18 | 85.24 ± 0.14 | 89.95 ± 0.56 |
| F17 | 87.29 ± 0.24 | 86.30 ± 0.67 | 90.09 ± 0.29 |
| F18 | 87.75 ± 0.30 | 87.78 ± 0.24 | 90.07 ± 0.44 |

For samples stored at 5° C., there is no measurable loss of purity (Table 45), with all of the values being essentially unchanged from the initial values within the error of the method. For samples stored at 25° C., there were some small losses by RP HPLC at t13 and t22 (Table 44). Still, the error in the RP method made it difficult to determine how sizable these losses really were. In general, the losses ranged from 2 to 4% over five months, indicating that the chemical stability of albiglutide is quite good in these formulations.

### PLS Modeling of SEC and RP Data

A number of PLS models were constructed using these data as endpoints. They are listed in order of the time points used, with the models for the three and five month time points at the end of this section.

A PLS1 model was constructed using the difference in monomer content at t2/40° C. as the endpoint. The model employed one PC and had a correlation coefficient for the calibration set of 0.865 and 0.751 for the validation set. This indicates that the model is not as robust as some of the PLS models for the Study 1.2 data. Five factors were calculated as being significant, citrate, protein, arginine, trehalose, and PS 80. The response surface for pH and citrate shows that the loss of monomer content is lowest at pH 6 and with the highest concentration of citrate. As far as the protein concentration effects, the response surface is relatively flat for concentrations up to ˜70 mg/mL, with slightly higher losses above that threshold. The increased loss at 100 mg/mL is predicted to be about 0.4% after two weeks over a lower concentration sample (cf. discussion below).

A certain formulation contains 100 mM arginine and 117 mM trehalose. One primary goal of Study 1.3 was to investigate the effect of changing these amounts. The response surface for arginine and trehalose shows a dome shape, where the general trend is that arginine is destabilizing while trehalose is stabilizing at pH 6. There is a less abrupt difference when samples are stored at lower temperatures, possibly due to the fact that hydrophobic effects tend to be magnified at elevated temperatures. Finally, it appears that PS 80 is a stabilizer in this PLS model.

A second PLS1 model for the Study 1.3 data was constructed using the monomer content after four weeks at 25° C. (t4/25° C.) as the endpoint. The model employed one PC and had a correlation coefficient for the calibration set of 0.811 and 0.636 for the validation set. This model reflects data at a lower storage temperature and thus may be more relevant in terms of long-term storage stability.

The response surface for pH and citrate indicates the optimal pH is near 5.7 to 5.8, with citrate appearing to be a good stabilizer.

In order to judge the magnitude of the effect of protein concentration, the predicted monomer content percentage with PS 80 is listed on the surface. It shows about a 0.15% decrease in monomer content by moving from 30 mg/mL to 100 mg/mL. The trend is entirely consistent with all of the rest of the analyses performed to date. Higher protein concentrations do lead to decreased stability. At the same time, PS 80 appears, as it does in this model, to provide a modicum of stabilization.

A third PLS model was constructed using the difference in dimer content at t2/30 C, t3/30 C, and t4/25 C. While all of these temperatures reflect accelerated stress conditions, they do provide information on the ability of albiglutide to survive temperature excursions. In addition, by considering multiple time points and temperatures, a more balanced view of the stability profile may emerge. In all of the PLS models described in this report, the data are centered and normalized to the inverse of the standard deviation. Therefore, any response can be used as an endpoint with equal weighting to any other. Therefore, stability data from two different temperatures can be used in conjunction with each other, with each one given equal consideration.

This PLS2 model has two outliers (formulations 14 and 15), but produced a high quality model with a calibration r-value of 0.958 and 0.905 for the validation set, using three PCs. It is not clear why they exhibit data that are inconsistent with the others, except that they do represent extremes in the trehalose and Arginine concentrations.

The optimal pH according to this model is 5.9 to 6.0, with citrate being slightly destabilizing at 15 mM. These results are consistent with earlier conclusions that pH 6.0 is nearly ideal and a modest amount of citrate (10 to 15 mM) needs to be present.

The protein concentration impacts the stability, according to this model, but only at concentrations above 70 to 80 mg/mL or higher. Again, this finding is consistent with data that have been gathered for Study 1.2 and Study 1.3. Each data set points to the fact that concentrations about 60 mg/mL are likely to reduce the storage stability of albiglutide.

The data and these models suggest that variations in 20 mM for either arginine or trehalose will have a minimal impact on stability as measured by SEC. In other words, the arginine concentration could range from 80 to 120 mM and trehalose could vary from 100 to 140 mM. Once again, response surfaces indicate that PS 80 is a modest stabilizer, at least according to this model, although this seems to be the general trend.

As data were collected for samples stored out to five months (22 weeks), a PLS2 model was constructed using the monomer content at t13 and t22 for samples stored at 25° C. as the endpoints. The model had a correlation coefficient for the calibration set of 0.856 and an r-value for the validation set of 0.775. Three factors were deemed to be statistically significant, which were protein concentration, citrate, and PS 80.

The response surface for pH and citrate shows that monomer content is maximal at the highest citrate level, with a slight preference for pH 5.7 over higher pH values. The protein concentration has a significant effect on stability in this model, the monomer content is constant until ˜60 mg/mL. Above that value, the stability does decrease according to this model. By comparison, arginine has relatively little effect on stability across the range of concentrations evaluated. Trehalose and arginine have little effect once the pH and citrate levels are fixed (note the range over the entire response surface). Finally, the effect of PS 80 was found to be beneficial for maintaining monomer content at 25° C.

A PLS2 model was constructed using the monomer content at t13 and t22 for samples stored at 5° C. as the endpoints. The model had a correlation coefficient for the calibration set of 0.938 and an r-value for the validation set of 0.604. Two factors were deemed to be statistically significant, which were protein concentration and citrate.

This model for samples stored at lower temperature, like the previous model finds citrate is a stabilizer and that there is a preference for pH 5.7 over higher pH values. The next response surface shows that monomer content is maintained at lower protein concentrations (<60 mg/mL), but lost as the concentration rises to 100 mg/mL. In addition, it appears that PS 80 may be beneficial in maintaining monomer content. This model indicates that arg and trehalose have little effect on the stability, within these ranges, much like the previous model. Suitably, the liquid compositions of the present invention may contain concentrations of 100 mM for arginine and 117 mM for trehalose.

A PLS1 model was constructed using the purity by RP HPLC at t22 for samples stored at 25 C as the endpoint. The losses at 5 C were too small to build a comparable model. This PLS model had a correlation coefficient of 0.859 for the calibration set and an r-value for the validation set of 0.700. Two factors were found to be significant, which were pH and the protein concentration.

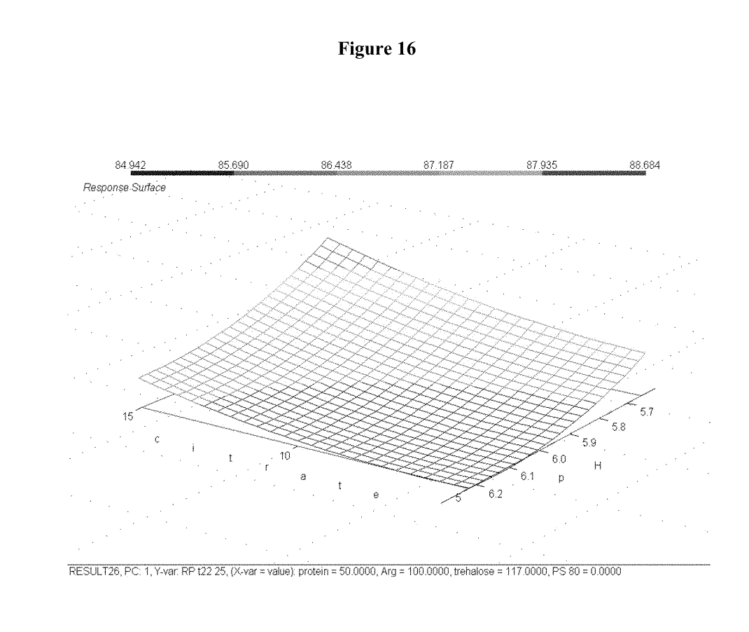

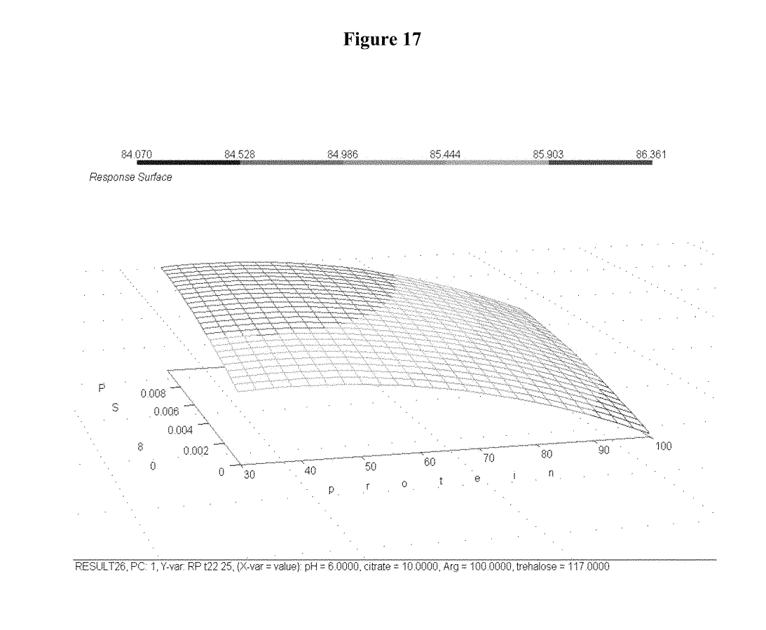

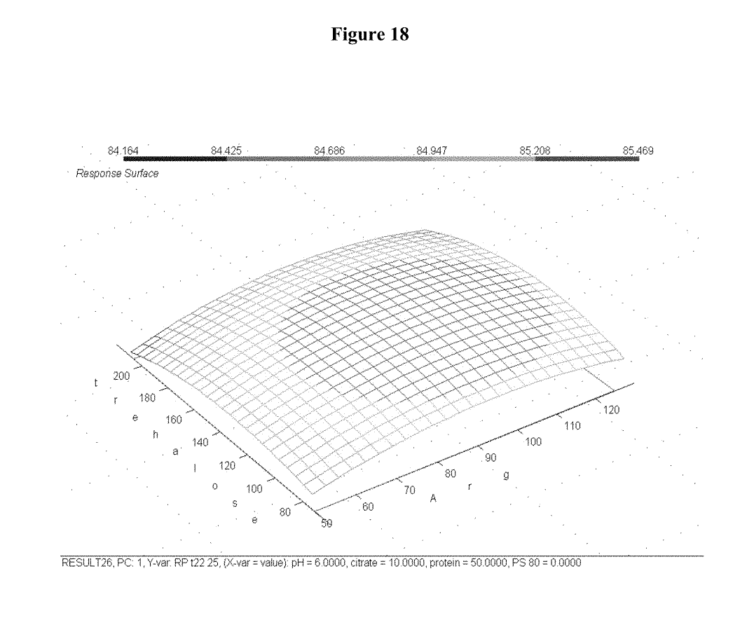

The effect of pH and citrate shows the same pattern as was seen for the SEC endpoints, where higher citrate is beneficial and pH 5.7 is preferred to maintain the purity of the protein at 25° C. (FIG. 16). In addition, higher protein concentration leads to loss of purity above a threshold value near 60 mg/mL, according to this model (FIG. 17). Addition of PS 80 appears to lead to greater retention of purity upon storage. The third response surface shows the effect of arginine and trehalose. The darker gray area top left quarter indicates the area of maximal stability. The current composition of 100 mM arginine and 117 mM trehalose falls directly in the middle of this region (FIG. 18). This data also suggests that changes in the levels of either stabilizer of 20% or more would not have an appreciable impact on stability.

### Supplemental Data

Samples from this Study 1.3 were used to generate supplemental data. In addition to SEC and RP, data have been collected using Caliper and cIEF. This section of the report provides a brief summary of the data, especially as they pertain to the samples stored for three and five months (t13 and t22).

TABLE 46
Caliper data for albiglutide samples from Study 1.3 stored for 22 weeks
(five months), with the reduced samples presented in bolded text and
the non-reduced samples listed in non-bolded black

| Form No | t22 5 C. | t22 25 C. | t0 | t22 5 C. | t22 25 C. |
|---|---|---|---|---|---|
| 1 | 99.9 | 99.7 | 97.6 | 98.3 | 96.9 |
| 2 | 99.9 | 99.7 | 97.6 | 98.3 | 96.8 |
| 3 | 99.9 | 99.7 | 97.6 | 98.1 | 96.8 |
| 4 | 99.9 | 99.7 | 97.8 | 98.0 | 97.0 |
| 5 | 99.9 | 99.7 | 97.6 | 98.2 | 97.1 |
| 6 | 99.9 | 99.7 | 97.8 | 98.2 | 97.1 |
| 7 | 99.9 | 99.7 | 97.6 | 97.9 | 97.4 |
| 8 | 99.9 | 99.7 | 97.3 | 98.0 | 97.3 |
| 9 | 99.9 | 99.7 | 97.2 | 98.1 | 97.2 |
| 10 | 99.9 | 99.7 | 97.3 | 97.9 | 97.0 |
| 11 | 99.9 | 99.7 | 97.5 | 98.2 | 97.2 |
| 12 | 99.9 | 99.7 | 97.6 | 98.5 | 97.2 |
| 13 | 99.9 | 99.7 | 97.4 | 98.4 | 97.1 |
| 14 | 99.9 | 99.7 | 97.3 | 98.3 | 97.1 |
| 15 | 99.9 | 99.7 | 97.4 | 98.4 | 97.2 |
| 16 | NT | | | | |
| 17 | 99.9 | 99.7 | 97.5 | 98.4 | 97.2 |
| 18 | | | | | |

NT = not tested

The samples containing PS 80 were not analyzed using the Caliper method, but the results for the remaining 16 formulations are listed in Table 46. The reduced samples, listed in bold type, show virtually no loss for any of the formulations after five months. The non-reduced method appears to be more stability-indicating, with some variation seen in the values across formulations. Still, the losses are so small that no further analysis of stability can be deduced from this technique.

TABLE 47
Percentage monomer content by SEC for formulations in
Study 1.3 stored 22 weeks (5 months) at 5° C. and 25° C.

| Form No | t0 | t22 5 C. | t22 25 C. |
|---|---|---|---|
| 1 | 98.3 | 98.0 | 96.8 |
| 2 | 98.4 | 98.1 | 96.8 |
| 3 | 98.3 | 98.0 | 97.2 |
| 4 | 98.2 | 97.9 | 97.0 |
| 5 | 98.3 | 98.0 | 97.2 |
| 6 | 98.4 | 98.0 | 97.1 |
| 7 | 98.3 | 97.9 | 96.9 |
| 8 | 98.2 | 97.8 | 96.8 |
| 9 | 98.1 | 97.7 | 96.7 |
| 10 | 98.3 | 98.0 | 97.1 |
| 11 | 98.3 | 97.9 | 96.9 |
| 12 | 98.2 | 97.9 | 96.9 |
| 13 | 98.3 | 98.0 | 97.0 |
| 14 | 98.2 | 97.9 | 96.7 |
| 15 | 98.2 | 97.9 | 97.0 |
| 16 | 98.2 | 97.8 | 96.6 |
| 17 | 98.1 | 97.7 | 96.5 |
| 18 | 98.1 | 97.8 | 96.7 |

The monomer content at t22 by SEC is summarized in Table 47. The initial values were near 98.3%. After five months at 5° C., those values are still mostly near 98%, meaning less than 0.1% is lost per month. By comparison, the levels at 25° C. are near 97%, which still only amounts to about 0.2% per month loss in monomer. Given the current specification of >96% monomer, all of these samples still would pass this test.

A PLS1 model was constructed using these data for samples stored for five months at 5° C. as the endpoint. The model had a correlation coefficient of 0.929 for the calibration set and a r-value of 0.700 for the validation set. Three factors were determined to be statistically significant, which were pH, citrate, and protein concentration.

The model indicates that citrate is a stabilizer and that the monomer content is maximized by going to pH 5.7 as opposed to higher pH values. Meanwhile, the monomer content remains highest at protein concentrations below 50 mg/mL. Addition of PS 80 is predicted to provide some modest amount of stabilization. The effects of arginine and trehalose appear to be maximal near the current target of 100 mM arginine and 117 mM trehalose. The model indicates that there is some significant latitude in these concentrations, where variations by more than 20 mM in each direct can be easily accommodated without impacting stability.

TABLE 48
Percentage dimer content by SEC for formulations in Study
1.3 stored 22 weeks (5 months) at 5° C. and 25° C.

| Form No | t0 | t22 5 C. | t22 25 C. |
|---|---|---|---|
| 1 | 1.7 | 2.0 | 3.2 |
| 2 | 1.6 | 1.9 | 3.2 |
| 3 | 1.7 | 2.0 | 2.8 |
| 4 | 1.8 | 2.1 | 3.0 |
| 5 | 1.7 | 2.0 | 2.8 |
| 6 | 1.6 | 2.0 | 2.9 |
| 7 | 1.7 | 2.1 | 3.1 |
| 8 | 1.8 | 2.2 | 3.2 |
| 9 | 1.9 | 2.3 | 3.3 |
| 10 | 1.7 | 2.0 | 2.9 |
| 11 | 1.7 | 2.1 | 3.1 |
| 12 | 1.8 | 2.1 | 3.1 |
| 13 | 1.7 | 2.0 | 3.0 |
| 14 | 1.8 | 2.1 | 3.3 |
| 15 | 1.8 | 2.1 | 3.0 |
| 16 | 1.8 | 2.2 | 3.4 |
| 17 | 1.9 | 2.3 | 3.5 |
| 18 | 1.9 | 2.2 | 3.3 |

The dimer contents for samples stored for 22 weeks (t22, five months) are summarized in Table 48. At 5° C., the levels are between 1.9% and 2.3%, only slightly elevated from the initial values near 1.7%. At 25° C., the dimer content has increased to 3% or more, but still below the current specification of <4%.

A PLS2 model was constructed using dimer levels for samples stored for five months at 5° C. and 25° C. as the endpoints. The model had a correlation coefficient of 0.961 for the calibration set and a r-value of 0.778 for the validation set. Three factors were determined to be statistically significant, which were pH, citrate, and protein concentration.

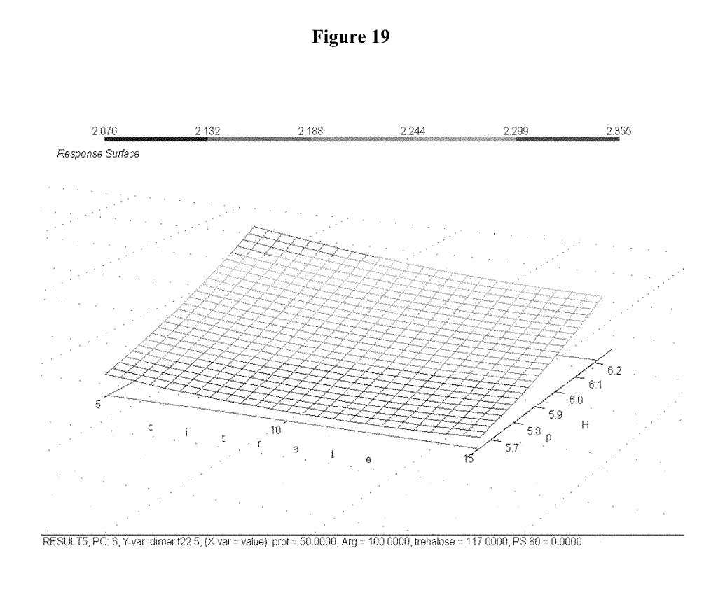

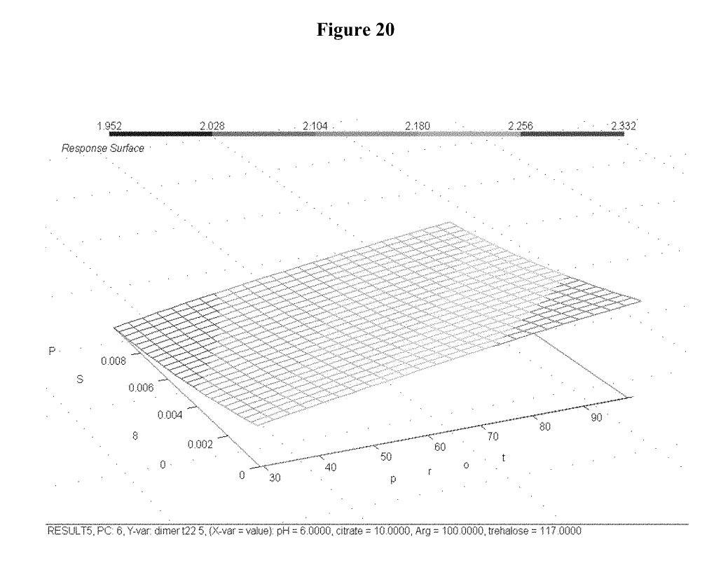

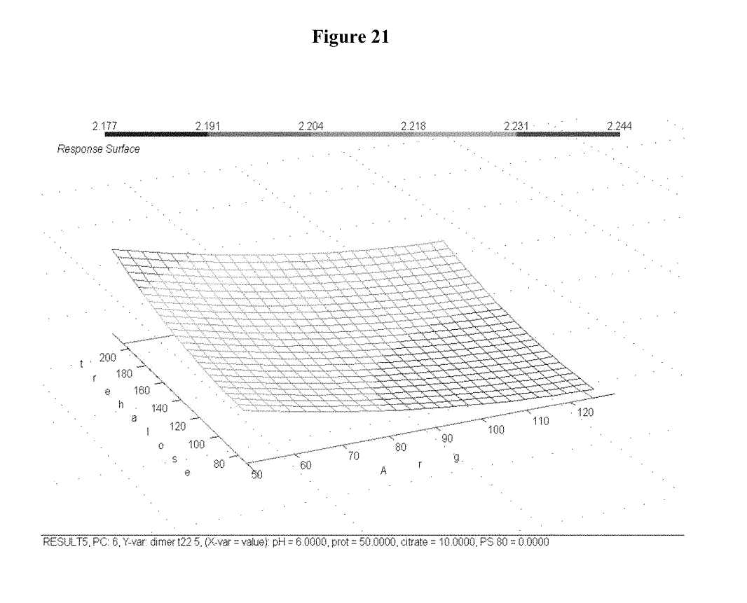

The PLS2 model indicates that citrate is a stabilizer, with the minimal dimer levels occurring at pH 5.7 (FIG. 19) These findings are consistent with the many PLS models described above, especially for those focusing on data for samples stored for at least three months. PS 80 is predicted to be a stabilizer, while increased protein concentrations are shown to lead to higher dimer levels (FIG. 20). The model shows that arginine concentrations above 90 mM and trehalose levels between 80 and 140 mM will minimize dimer formation (FIG. 21).

TABLE 49
Purity by RP HPLC conducted at GSK for Study 1.3 samples stored for 22
weeks at 5° C. and 25° C. Data for t0 are provide for comparison.

| Form No | t0 | t22 5 C. | t22 25 C. |
|---|---|---|---|
| 1 | 96.5 | 96.5 | 93.3 |
| 2 | 96.5 | 96.3 | 93.4 |
| 3 | 96.7 | 96.4 | 93.2 |
| 4 | 96.5 | 96.5 | 93.1 |
| 5 | 96.4 | 96.5 | 93.4 |
| 6 | 96.6 | 96.4 | 93.4 |
| 7 | 96.9 | 96.4 | 93.2 |
| 8 | 96.4 | 96.5 | 93.3 |
| 9 | 96.7 | 96.5 | 93.3 |
| 10 | 96.5 | 96.5 | 93.6 |
| 11 | 96.4 | 96.6 | 93.6 |
| 12 | 96.6 | 96.5 | 93.0 |
| 13 | 96.6 | 96.4 | 93.4 |
| 14 | 96.5 | 96.5 | 93.2 |
| 15 | 96.4 | 96.5 | 93.1 |
| 16 | | | |
| 17 | 96.5 | 96.5 | 93.4 |
| 18 | NT | NT | NT |

NT = not tested

The purity of the samples at t22 was measured using RP HPLC (Tale 49). The initial purities were near 96.5%. After five months at 5 C, these values were essentially unchanged. Even after 22 weeks at 25 C, the levels had decreased by only ˜3% (Table 49).

The final analytical test performed at GSK on these 18 formulations was to measure the charge profile using cIEF. The purity, as judges by the main peak intensity, is summarized for each formulation in Table 50. There were small decreases seen at t22 for the 5° C. samples and losses ranging from 7 to 11% for the samples stored at 25° C.

TABLE 50
Main peak purity by cIEF performed at GSK for Study 1.3
samples stored at 5° C. and 25° C. for 22 weeks

| Form No | t0 | t22 5 C. | t22 25 C. |
|---|---|---|---|
| 1 | 76.8 | 74.4 | 67.7 |
| 2 | 77.0 | 73.8 | 66.6 |
| 3 | 75.9 | 73.6 | 68.7 |
| 4 | 75.5 | 72.4 | 67.2 |
| 5 | 76.3 | 74.2 | 68.3 |
| 6 | 75.6 | 74.8 | 67.9 |
| 7 | 75.1 | 73.7 | 69.5 |
| 8 | 74.9 | 74.1 | 66.5 |
| 9 | 75.0 | 73.1 | 68.2 |
| 10 | 76.4 | 72.0 | 66.9 |
| 11 | 75.3 | 73.0 | 67.3 |
| 12 | 75.4 | 73.2 | 67.1 |
| 13 | 75.5 | 73.3 | 66.0 |
| 14 | 76.4 | 73.8 | 66.2 |
| 15 | 75.9 | 74.3 | 67.1 |
| 16 | 73.0 | 72.1 | 64.7 |
| 17 | 75.8 | 73.6 | 66.2 |
| 18 | 73.7 | 71.7 | 63.8 |

A PLS1 model was constructed using main peak purity by cIEF for samples stored for five months at 5° C. as the endpoint. The model had a correlation coefficient of 0.891 for the calibration set and a r-value of 0.663 for the validation set. Three factors were determined to be statistically significant, which were pH, citrate, and PS 80.

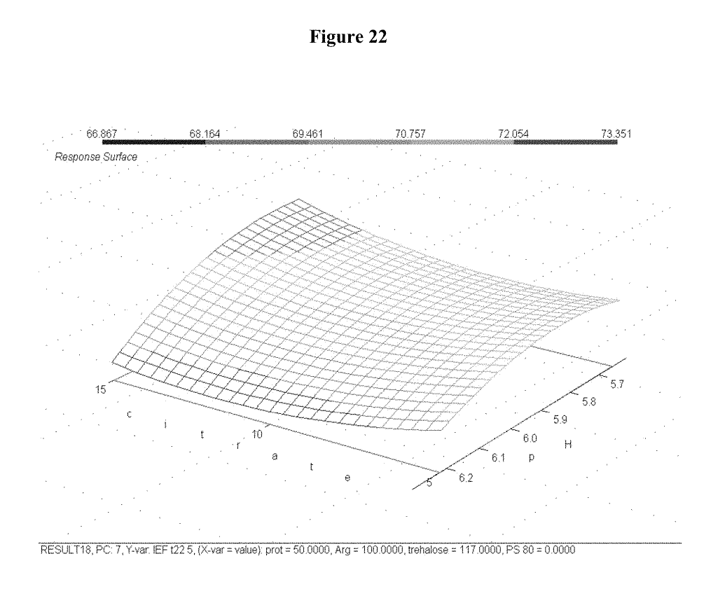

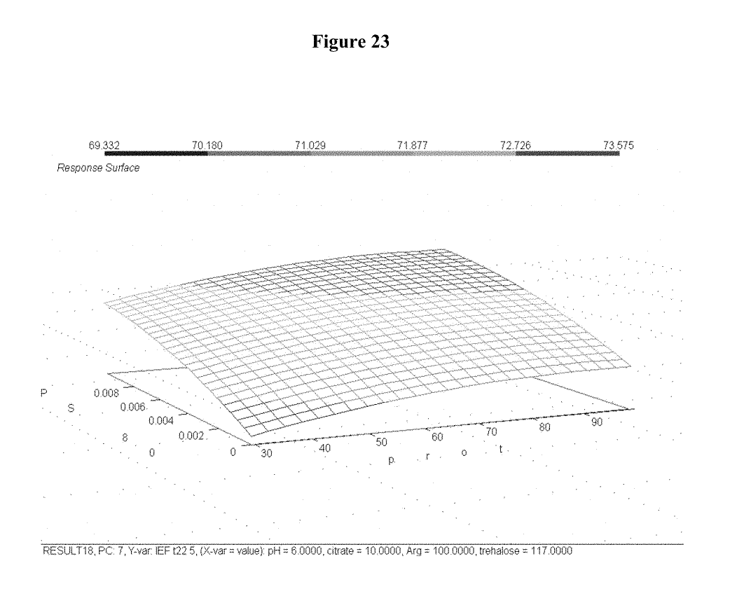

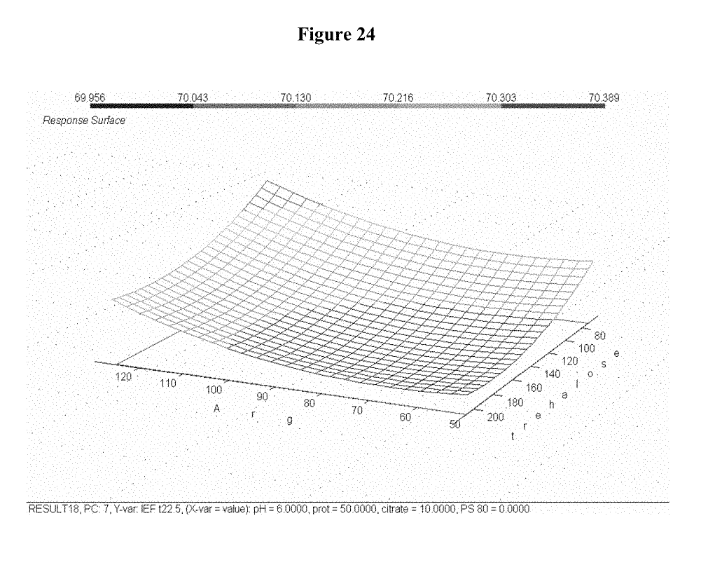

The PLS model indicates that citrate is effective at maintaining the main peak purity for these t22 samples, while pH 5.7 is also beneficial compared to higher pH values (FIG. 22). At the same time, addition of PS 80 is predicted to be beneficial (FIG. 23). The protein concentration dependence is slightly different than in previous models, where slightly higher concentrations are predicted to be helpful, but the response surface is fairly shallow in this area. Finally, the effect of arginine and trehalose is shown in FIG. 24. The entire range of the response surface is only about 0.4%, indicating that the amounts currently being used are nearly ideal. Even large changes are predicted to have minimal effect on stability according to this model.

### SUMMARY

Four sets of experiments have been performed to investigate the stability of albiglutide in aqueous solution. The primary stability-indicating assays are SEC and RP HPLC. Although cIEF and SDS-PAGE can be informative, they appear to lack the sensitivity of the HPLC to assess stability, at least for relatively stable formulations. Only cIEF was found to provide useful information on stability, and only after the samples were stored for five months.

The initial study, Study 1.0, indicated that histidine (His) formulations were less stable, as were phosphate formulations to a lesser extent. In addition, higher pH (>6.3) caused some instability as well. At the same time, these formulations were all quite resistant to freeze-thaw damage. Study 1.1 explored the buffer effects in greater details, adding succinate in place of His. It proved to be a possible alternative to citrate, but when everything was considered, citrate has continually been shown to be the best buffer for albiglutide.

Study 1.2 examined the effects of other components, such as mannitol, trehalose, and PS 80. The data showed once again that succinate was not nearly as stabilizing as citrate and that the pH optimum was from 5.5 up to 6.0. Along with the information from Study 1.3, it was found that one must exercise caution in selecting the buffer concentration. Repeatedly, the data point to a modest level of citrate, about 10 to 15 mM, as being the best range. As with other proteins, it appears that albiglutide is destabilized by an excess of citrate, likely due to reduced colloidal stability. Octanoate was found to be destabilizing, while the effects of mannitol and trehalose were similar and, for the most part, relatively weak.

The optimization of the formulation continued in Study 1.3, again investigating trehalose and mannitol, along with varying the arginine and PS 80 levels. It does appear from Study 1.3 (to date) that PS 80 is a stabilizer. Even thought the effects tend to be modest, the consistent observation is hat having PS 80 present is beneficial.

The pH and buffer selection was finalized with Study 1.3. The optimal pH appears to be 5.7, especially when one considers the data from the three and five month time points. Yet, in general, the overall tenor of these studies has shown that the response to pH is relatively flat across this limited pH range of 5.7 to 6.2. Still, the latest data make a compelling argument for selecting pH 5.7, as this seems to stabilize the protein whether stability is determined by SEC, RP HPLC or cIEF. As stated above for earlier studies, a modest amount of citrate (10 to 15 mM) appears to be ideal.

The results from Study 1.3 indicate that the formulation which employs 100 mM arginine and 117 mM trehalose is well chosen. Variations of about 20 mM (or more) for either compound appear to have a minimal impact on stability, as measured by SEC, RP and even cIEF. In other words, the arginine concentration could range from 80 to 120 mM and trehalose could vary from 100 to 140 mM.

The effects of protein concentration were explored in greater detail in Study 1.3, building on findings from Study 1.2. It was found that below ˜60 mg/mL the stability profile was relatively flat, but higher protein concentrations did lead to loss of monomer. Some decreased stability at higher protein concentrations was also observed by RP HPLC. Therefore, when considering long-term storage, a concentration of 50 mg/mL to 60 mg/mL would be the highest one could use without compromising stability and still allow greater ease of dosing.

Although there are some differences observed through the various studies, the composition of the optimal liquid composition has emerged. The formulation should be at about pH 5.7, where a variation in pH of possibly 0.3 units could be reasonably well tolerated. A citrate concentration of 10 to 15 mM is best as well as a formulation that contains 100 mM arginine and 117 mM trehalose. Overall, the data indicate that these levels are not far from being optimal. For the most part, PS 80 at 0.01% or so appears to be beneficial for samples stored at elevated temperatures.

Storage at 40° C. does produce higher MW aggregates that are not observed upon storage at 25° C. or even 30° C. Still, the loss of monomer by SEC does appear to correlate roughly between the different storage temperatures. In no cases was any fragmentation seen, only formation of dimer, oligomer and higher MW aggregate. When examining the purity of these formulations by RP HPLC, very little, if any, decrease in purity is seen at 5° C. Even at 25 C, the changes in purity are less than 5% after five months. Likewise, cIEF data indicates that less than 10%, at most, of the main peak purity is lost at 25° C. after five months, with less than 5% lost for 5° C. storage. These findings suggest that very little chemical degradation is occurring in albiglutide upon storage, with the primary degradation pathway being formation of soluble aggregates. Even this can be largely arrested by proper formulation and storage at 2-8° C.

### Example 2

Albiglutide Bulk Drug Substance (BDS) has been manufactured via Process 3 (P3) from Route of Synthesis B3. Heat treatment has been explored as a possible step in P3 and has been shown by Downstream Processing Development (DPD) to reduce protease activity and further enzymatic degradation of albiglutide leading to highly potent peptides as determined by bioassay and RPHPLC. Reduction of the proteolytic degradation of albiglutide could potentially make a solution formulation feasible. In addition to the proteolytic degradation, aggregation and oxidation are the two main mechanism of degradation for albiglutide in solution. Studies indicated that acidic variant, mainly peak 97 in cIEF is N-terminal and lysine carbamylation, cysteine and tryptophan oxidation. Studies showed that albiglutide is more stable against solution aggregation in a formulation comprising 5 mM citrate, 100 mM arginine hydrochloride, and 117 mM trehalose at pH 5.7. This formulation is termed the ‘optimized’ formulation in this example. Previous studies also showed that addition of sodium octanoate to the current lyophile formulation (pH 7.2, 10 mM phosphate, 153 mM mannitol, 117 mM trehalose, and 0.01% (w/w) PS80) can stabilize albiglutide from an alternative manufacturing process, Process 2 (P2), against aggregation.

In this study, the solution stability of P3 and heat-treated P3 material in different formulations was investigated. In addition, diafiltration buffer pH around albiglutide pI effect on aggregation was investigated.

The objectives of this study were:
1. Compare the stability of P3 material and heat-treated P3 material
2. Compare the current lyophilized formulation and optimized solution formulation
3. Compare P2 and P3 for non-heat treated albiglutide
4. Evaluate the effect of pH (5.7 and 6.5) for the optimized solution formulation
5. Determine whether methionine can help to prevent the oxidation of albiglutide
6. Determine if octanoate has the same stabilizing effect on P3 material as it does on P2 material in solution
7. Determine if octanoate can stabilize albiglutide in optimized solution formulation at pH 6.5
8. Evaluate the effect of pH on the aggregation of albiglutide during diafiltration

### Experiment Set Up and Assay

### Materials:

Albiglutide: P3 and P3 heat-treated materials in optimized solution formulation and current lyophile formulation were obtained from DPD. 15 L of Blue SAHL pool from PD3 large-scale run was further purified by DPD. Another 15 L (3×5 L cycles) was heat treated and purified. Since this material was processed by DPD at small scale, it may not be representative of material manufactured at full scale.

Albiglutide diafiltration samples were prepared through UFDF step to change the buffer to 5 mM citrate, 100 mM arginine hydrochloride, 117 mM trehalose at pH 5.0, 5.7 and 6.5. Samples at pH 5.0, 5.7 and 6.5 were prepared in the first diafiltration, however, sample in pH5.7 was diafiltrated in water at the beginning, then pH 5.7 buffer was used, and high order aggregates (about 0.8%) were observed in the diafiltration sample. Diafiltration in pH 5.7 was repeated.

Vials: 3 mL glass vial, glass type I, untreated, GSK Comet
Flurotec serum stoppers: 13 mm, injection 1358, West 4023/50, gray, GSK Comet

### Formulations:

See Table 51 for formulation information.

TABLE 51
Formulation Information

| | Protein | | |
|---|---|---|---|
| Formu- | concent- | | Material and |
| lation | ration (mg/mL) | Formulation | preparation |
| 1 | ~80 | pH 7.2, 10 mM phosphate, 153 mM mannitol, | P3 from DPD |
| | | 117 mM trehalose, and ~0.01% (w/w) PS80 | |
| 2 | ~80 | pH 7.2, 10 mM phosphate, 153 mM mannitol, | P3 heat-treated |
| | | 117 mM trehalose, and ~0.01% (w/w) PS80 | from DPD |
| 3 | ~80 | pH 5.7, 5 mM citrate, 100 mM arginine | Dialysis from P3 |
| | | hydrochloride, 117 mM trehalose, and | heat-treated |
| | | ~0.01% (w/w) PS80 | |
| 4 | ~80 | pH 6.5, 10 mM phosphate, 100 mM arginine | Dialysis from P3 |
| | | hydrochloride, 117 mM trehalose, and | heat-treated |
| | | ~0.01% (w/w) PS80 | |
| 5 | ~80 | pH 6.5, 10 mM phosphate, 100 mM arginine | Dialysis from P3 |
| | | hydrochloride, 117 mM trehalose, 10 mM | heat-treated |
| | | methionine (Met), and ~0.01% (w/w) PS80 | |
| 6 | ~80 | pH 7.2, 10 mM phosphate, 153 mM mannitol, | P3 from DPD + |
| | | 117 mM trehalose, 10 mM sodium octanoate, | octanoate |
| | | and ~0.01% (w/w) PS80 | |
| 7 | ~80 | pH 6.5, 10 mM phosphate, 100 mM arginine | Dialysis from P3 + |
| | | hydrochloride, 117 mM trehalose, 10 mM | octanoate |
| | | sodium octanoate, and ~0.01% (w/w) PS80 | |
| 10 | ~120 | pH 7.2, 10 mM phosphate, 153 mM mannitol, | P3 from DPD |
| | | 117 mM trehalose, and ~0.01% (w/w) PS80 | |
| 11 | ~120 | pH 7.2, 10 mM phosphate, 153 mM mannitol, | P2 |
| | | 117 mM trehalose, and ~0.01% (w/w) PS80 | |

### Sample Preparation and Set Down

Samples were dialyzed in the target formulation 3 times at 2-8° C. After dialysis the samples were diluted to 80 mg/mL in the target formulation to match the concentration of the sample in optimized formulation from DPD (P3 heat-treated in optimized formulation). Samples were prepared and set down at the following temperatures as shown in Table 52.

TABLE 52
Stability testing schedule

| Temperature | Time 0 | 1 month | 2 month | 3 month | 6 month | 12 month |
|---|---|---|---|---|---|---|
| 2-8° C. | √ | √ | | √ | √ | √ |
| 25° C./60% RH | √ | √ | | √ | √ | |
| 40° C./75% RH | √ | √ | SEC, | | | |
| | | | cIEF | | | |

Fill volume: 0.5 mL

### Assay Method and Acceptance Criteria

Samples at time 0 were analyzed by capillary DSC. Samples were diluted to 1 mg/mL with the same formulation buffer as each sample. The scan rate was 1° C./min. Scan starting temperature was 25° C., ending temperature was 95° C. For bioassay, formulation 1 and 2 were analyzed in triplicate at time 0. For all other time points, a single measurement was performed.

TABLE 53
Testing method and acceptance criteria

| | | Stability Acceptance Criteria for 50 |
|---|---|---|
| Test | Testing Method | mg/Pen (AAY) based on STD_135403_2.0 |
| Appearance and | INS_8930¹ | Clear to opalescent, essentially particle |
| Description | | free solution. Report color and color |
| | | grade. |
| pH | Ph.Eur. 2.2.3 and | 6.6-7.5 for pH 7.2 formulations, for |
| | USP <791> | information for other pH targets |
| Size Exclusion | INS_8898 | Monomer ≥ 95.0% |
| Chromatography (SEC) | | % Aggregate: Report results (1 dp) |
| Reverse-Phase HPLC | INS_10015 | ≥84.0% Main peak |
| Capillary Isoelectric | INS_8922 | ≥59.0% Main |
| Focusing (cIEF) | | pI Main: Report result (2 dp) |
| Protein concentration | INS_109290 | For information only |
| Capillary Gel | INS_107709 | Reduced: ≥95.0% |
| Electrophoresis (CGE) | | Non-Reduced: ≥95.0% |
| Reduced and non-reduced | | |
| Osmolality | INS_70585 | Report Results, mOsm/kg (0 dp) |
| Bioassay² | INS_8838 | 50.0-150.0% Relative Potency |
| Capillary DSC | INS_60971 | Report Results |

Samples at 40° C. for 8 weeks were only analyzed for SEC-HPLC and cIEF.
Formulation 1 at 2-8° C. for 12 months was not assayed due to shortage of the sample.

### Results

### Stability of Albiglutide in Different Formulations

Table 54 shows the transition temperatures, ΔH and thermal unfolding curves of albiglutide in different formulations at time 0 as measured by capillary DSC. Tables 55-57 show the results of albiglutide in different formulations after storage at 2-8° C. up to 12 months, at 25° C. up to 6 months and at 40° C. up to 8 weeks. Stability of albiglutide in different formulations at 2-8, 25 and 40° C. measured by pH, protein concentration by RP-HPLC, SEC, RP-HPLC, cIEF, CGE and Osmolality is shown in Table 55. For 40° C. samples, graphs are drawn for data with 3 time points (SEC-HPLC and cIEF); graphs are not drawn for data with 2 time points.

The results of albiglutide in different formulations at 2-8 and 25° C. measured by SEC-HPLC, RP-HPLC, cIEF and CGE non-reduced (NR) and reduced (R) were linearly trended using SigmaPlot version 10 to determine if statistically significant degradation occurred. The other assays were not included in statistical analysis since no significant differences between the initial and final time points were observed. The p-value and slope for each assay at 2-8 and 25° C. are shown in Table 58. The slope of the linear regression corresponds to the degradation rate. Slopes were considered statistically significant if the p-value was ≤0.1. The degradation rate was reported for the assays if the p-value was ≤0.1. The degradation rate was reported as 0 if the p-value was >0.1. The plots of raw data versus time for assays with statistically significant slope are contained in the Appendix 3.

TABLE 54
Transition Temperatures of albiglutide in Different
Formulations at Time 0 as Measured by Capillary DSC

| | Formulation | | | | | | |
|---|---|---|---|---|---|---|---|
| | 1 | 2 | 3 | 4 | 5 | 6 | 7 |
| Tonset (° C.) | 44.8 | 44.5 | 56.2 | 53.5 | 51.5 | 66.9 | 71.4 |
| Tm 1 (° C.) | 58.0 | 61.3 | 68.0 | 64.9 | 64.3 | 78.8 | 79.5 |
| Tm 2 (° C.) | 71.1 | 72.2 | 73.3 | 73.2 | 72.2 | 79.7 | 81.3 |
| ΔH1 | 91 | 93 | 130 | 97 | 100 | 180 | 46 |
| (kCal/Mole) | | | | | | | |
| ΔH2 | 100 | 130 | 130 | 110 | 130 | 170 | 280 |
| (kCal/Mole) | | | | | | | |

Note:
Tonset is the temperature protein starts to unfold. It is determined by visual examination of the unfolding profile to determine the temperature at which the Cp starts to increase dramatically.

TABLE 55
Stability Data at 2-8° C.

| | Time | | Formulation | | | | | | | | |
|---|---|---|---|---|---|---|---|---|---|---|---|
| Assay | (Month) | | 1 | 2 | 3 | 4 | 5 | 6 | 7 | 10 | 11 |
| General | 0 | | ¹GA | ²GA | ²GA | ²GA | ³GA | ³GA | ³GA | ⁶GA | ⁷GA |
| appearance | 1 | | ³GA | ²GA | ²GA | ²GA | ²GA | ³GA | ³GA | ⁶GA | ⁷GA |
| | 3 | | ²GA | ²GA | ²GA | ²GA | ²GA | ³GA | ³GA | ⁵GA | ¹⁰GA |
| | 6 | | ²GA | ²GA | ²GA | ⁵GA | ²GA | ²GA | ²GA | NT | NT |
| | 12 | | NT | ²GA | ²GA | ²GA | ²GA | ²GA | ²GA | NT | NT |
| pH | 0 | | 7.1 | 7.1 | 5.9 | 6.4 | 6.4 | 7.1 | 6.4 | 7.2 | 7.2 |
| | 1 | | 7.1 | 7.1 | 5.9 | 6.4 | 6.4 | 7.1 | 6.5 | 7.1 | 7.0 |
| | 3 | | 7.2 | 7.1 | 6.0 | 6.5 | 6.5 | 7.2 | 6.6 | 7.1 | 7.0 |
| | 6 | | 7.1 | 7.1 | 6.0 | 6.4 | 6.5 | 7.2 | 6.5 | NT | NT |
| | 12 | | NT | 7.0 | 6.0 | 6.4 | 6.4 | 7.2 | 6.5 | NT | NT |
| Concentration | 0 | | 79.2 | 80.8 | 79.8 | 80.0 | 78.6 | 79.2 | 79.5 | 118.7 | 123.0 |
| (RP-HPLC | 1 | | 78.4 | 79.0 | 78.4 | 79.0 | 78.5 | 78.4 | 78.6 | 117.4 | 121.5 |
| mg/mL) | 3 | | 78.3 | 79.7 | 79.2 | 78.9 | 78.6 | 77.8 | 79.0 | 119.6 | 121.2 |
| | 6 | | 79.5 | 80.6 | 80.3 | 77.0 | 79.0 | 80.1 | 80.9 | NT | NT |
| | 12 | | NT | 81.7 | 79.9 | 79.9 | 80.0 | 79.9 | 80.7 | NT | NT |
| SEC-HPLC | 0 | Main | 98.0 | 97.8 | 97.9 | 97.8 | 97.8 | 98.0 | 98.0 | 97.9 | 98.7 |
| (%) | | ⁺Pk91 | 1.8 | 2.0 | 1.9 | 1.9 | 1.9 | 1.8 | 1.7 | 1.9 | 1.6 |
| | | ⁺Pk87 | 0.2 | 0.2 | 0.3 | 0.3 | 0.2 | 0.2 | 0.2 | 0.2 | 0.1 |
| | 1 | Main | 97.7 | 97.6 | 97.7 | 97.6 | 97.6 | 97.8 | 97.8 | 97.5 | 98.2 |
| | | ⁺Pk91 | 2.0 | 2.1 | 2.0 | 2.1 | 2.1 | 1.9 | 1.9 | 2.2 | 1.6 |
| | | ⁺Pk87 | 0.3 | 0.3 | 0.3 | 0.3 | 0.3 | 0.3 | 0.3 | 0.3 | 0.2 |
| | 3 | Main | 97.4 | 97.2 | 97.6 | 97.5 | 97.6 | 97.8 | 97.8 | 97.0 | 97.9 |
| | | ⁺Pk91 | 2.3 | 2.4 | 2.2 | 2.2 | 2.2 | 2.1 | 2.0 | 2.5 | 1.9 |
| | | ⁺Pk87 | 0.3 | 0.3 | 0.3 | 0.2 | 0.2 | 0.2 | 0.3 | 0.4 | 0.2 |
| | 6 | Main | 97.1 | 96.9 | 97.6 | 97.9 | 97.5 | 97.7 | 97.7 | NT | NT |
| | | ⁺Pk91 | 2.6 | 2.7 | 2.2 | 2.0 | 2.2 | 2.1 | 2.1 | NT | NT |
| | | ⁺Pk87 | 0.3 | 0.3 | 0.3 | 0.1 | 0.3 | 0.2 | 0.2 | NT | NT |
| | 1 | Main | NT | 96.9 | 97.7 | 97.6 | 97.6 | 97.7 | 97.8 | NT | NT |
| | 2 | ⁺Pk91 | NT | 2.8 | 2.1 | 2.2 | 2.1 | 2.1 | 2.0 | NT | NT |
| | | ⁺Pk87 | NT | 0.4 | 0.2 | 0.2 | 0.3 | 0.2 | 0.2 | NT | NT |

| | | | | | | | | | | |
|---|---|---|---|---|---|---|---|---|---|---|
| RP-HPLC | 0 | 92.6 | 92.3 | 92.5 | 92.4 | 92.5 | 93.1 | 93.2 | 93.1 | 92.3 |
| (% Main) | 1 | 94.1 | 94.0 | 93.8 | 94.1 | 93.8 | 94.1 | 94.0 | 93.8 | 92.4 |
| | 3 | 95.6 | 94.9 | 95.2 | 95.7 | 95.7 | 95.9 | 94.8 | 95.4 | 92.3 |
| | 6 | 94.5 | 94.1 | 94.8 | 94.0 | 94.6 | 94.2 | 93.8 | NT | NT |
| | 12 | NT | 95.0 | 95.3 | 94.8 | 95.5 | 94.6 | 93.5 | NT | NT |
| cIEF | 0 | 73.4 | 72.3 | 73.9 | 71.8 | 73.6 | 72.0 | 73.6 | 74.1 | 73.2 |
| (% Main) | 1 | 74.4 | 71.5 | 72.3 | 70.5 | 73.8 | 71.1 | 73.1 | 75.2 | 73.3 |
| | 3 | 74.0 | 72.9 | 73.2 | 72.9 | 74.2 | 73.4 | 73.2 | 73.1 | 68.0 |
| | 6 | 74.7 | 70.5 | 72.6 | 70.8 | 73.8 | 73.6 | 70.8 | NT | NT |
| | 12 | NT | 73.2 | 74.4 | 74.4 | 74.1 | 71.9 | 70.4 | NT | NT |
| CGE | 0 | 96.7 | 96.3 | 96.6 | 96.3 | 96.5 | 96.7 | 96.6 | 96.4 | 97.8 |
| (Non-reduced | 1 | 97.4 | 97.1 | 97.1 | 97.2 | 97.2 | 97.5 | 97.3 | 96.8 | 98.2 |
| % Main) | 3 | 96.8 | 96.7 | 97.0 | 96.9 | 97.0 | 97.1 | 97.0 | 96.3 | 96.3 |
| | 6 | 96.0 | 95.6 | 96.2 | 92.7^{Δ} | 96.3 | 96.6 | 96.6 | NT | NT |
| | 12 | NT | 96.8 | 97.3 | 97.3 | 97.1 | 97.3 | 97.4 | NT | NT |
| CGE | 0 | 99.0 | 98.9 | 98.9 | 98.9 | 98.9 | 99.0 | 99.0 | 99.1 | 99.3 |
| (Reduced | 1 | 99.6 | 99.5 | 99.5 | 99.5 | 99.5 | 99.6 | 99.6 | 99.5 | 99.6 |
| % Main) | 3 | 99.2 | 99.2 | 99.2 | 99.2 | 99.2 | 99.3 | 99.3 | 99.3 | 99.2 |
| | 6 | 98.6 | 98.4 | 98.9 | 96.3 | 98.6 | 98.9 | 98.8 | NT | NT |
| | 12 | NT | 99.4 | 99.6 | 99.5 | 99.6 | 99.6 | 99.6 | NT | NT |
| Osmolality | 0 | 355 | 339 | 332 | 342 | 356 | 365 | 357 | 378 | 369 |
| (mOsm/kg) | 1 | 351 | 345 | 334 | 345 | 350 | 369 | 359 | 380 | 369 |
| | 3 | 353 | 341 | 335 | 345 | 357 | 360 | 351 | 381 | 369 |
| | 6 | 365 | 340 | 326 | 351 | 355 | 359 | 353 | NT | NT |
| | 12 | NT | 347 | 335 | 344 | 353 | 366 | 356 | NT | NT |
| *Bioassay | 0 | 115.7 | 104.6 | NT | NT | NT | NT | NT | NT | NT |
| (% Relative | 1 | NT | NT | NT | NT | NT | NT | NT | NT | NT |
| potency) | 3 | 107.1 | 80.3 | 93.5 | 84.3 | 99.1 | 98.3 | 84.2 | NT | NT |
| | 6 | 112.3 | 116.0 | 83.3 | 240.4^{Δ} | 106.0 | 141.4 | 116.7 | NT | NT |
| | 12 | NT | 82.5 | 109.4 | 97.8 | 109.7 | 125.4 | 99.0 | NT | NT |

¹GA: clear, yellow (between Y1 and Y2 but more yellow [less brown]), essentially particle free solution
²GA: clear, yellow (between Y1 and Y2), essentially particle free solution
³GA: clear, yellow (Y2 or Y2 but slightly more yellow) essentially particle free solution
⁵GA: clear, yellow (Y1), essentially particle free solution
⁶GA: clear, yellow (slightly darker than Y1), essentially particle free solution
⁷GA: clear, yellow (darker than Y1), essentially particle free solution
¹⁰GA: clear, yellow (between CY2 and CY3), essentially particle-free solution
NT: Not Tested
^{Δ}: Sample contamination was observed after the sample was stored at the 2-8° C. over 7 days. The presence of a fragment peak was seen in CGE analysis.

TABLE 56
Stability Data at 25° C.

| | Time | | Formulation | | | | | | | | |
|---|---|---|---|---|---|---|---|---|---|---|---|
| Assay | (Month) | | 1 | 2 | 3 | 4 | 5 | 6 | 7 | 10 | 11 |
| General | 0 | | ¹GA | ²GA | ²GA | ²GA | ³GA | ³GA | ³GA | ⁶GA | ⁷GA |
| appearance | 1 | | ³GA | ³GA | ²GA | ³GA | ³GA | ³GA | ³GA | ⁸GA | ⁹GA |
| | 3 | | ³GA | ³GA | ²GA | ³GA | ³GA | ³GA | ³GA | ⁵GA | ¹⁰GA |
| | 6 | | ³GA | ²GA | ²GA | ²GA | ²GA | ²GA | ²GA | NT | NT |
| pH | 0 | | 7.1 | 7.1 | 5.9 | 6.4 | 6.4 | 7.1 | 6.4 | 7.2 | 7.2 |
| | 1 | | 7.1 | 7.1 | 5.9 | 6.5 | 6.4 | 7.1 | 6.5 | 7.1 | 6.9 |
| | 3 | | 7.1 | 7.1 | 6.0 | 6.5 | 6.5 | 7.2 | 6.6 | 7.1 | 7.0 |
| | 6 | | 7.1 | 7.1 | 6.0 | 6.5 | 6.5 | 7.1 | 6.5 | NT | NT |
| Concentration | 0 | | 79.2 | 80.8 | 79.8 | 80.0 | 78.6 | 79.2 | 79.5 | 118.7 | 123.0 |
| (RP-HPLC | 1 | | 78.2 | 79.1 | 78.8 | 78.4 | 78.3 | 79.2 | 78.9 | 117.1 | 121.1 |
| mg/mL) | 3 | | 78.6 | 79.8 | 78.9 | 78.8 | 79.1 | 78.4 | 78.8 | 118.5 | 119.2 |
| | 6 | | 80.1 | 80.7 | 80.8 | 79.9 | 79.7 | 80.3 | 80.5 | NT | NT |
| SEC-HPLC | 0 | Main | 98.0 | 97.8 | 97.9 | 97.8 | 97.8 | 98.0 | 98.0 | 97.9 | 98.7 |
| (%) | | ⁺Pk91 | 1.8 | 2.0 | 1.9 | 1.9 | 1.9 | 1.8 | 1.7 | 1.9 | 1.6 |
| | | ⁺Pk87 | 0.2 | 0.2 | 0.3 | 0.3 | 0.2 | 0.2 | 0.2 | 0.2 | 0.1 |
| | 1 | Main | 96.4 | 96.2 | 97.4 | 97.1 | 97.0 | 97.4 | 97.4 | 95.8 | 96.5 |
| | | ⁺Pk91 | 3.1 | 3.4 | 2.3 | 2.5 | 2.6 | 2.4 | 2.3 | 3.5 | 2.8 |
| | | ⁺Pk87 | 0.4 | 0.4 | 0.4 | 0.3 | 0.3 | 0.3 | 0.3 | 0.6 | 0.4 |
| | 3 | Main | 95.1 | 94.8 | 96.9 | 96.4 | 96.6 | 96.8 | 96.8 | 94.3 | 95.5 |
| | | ⁺Pk91 | 4.4 | 4.6 | 2.7 | 3.2 | 3.1 | 3.0 | 2.9 | 4.8 | 3.2 |
| | | ⁺Pk87 | 0.6 | 0.6 | 0.4 | 0.4 | 0.3 | 0.2 | 0.3 | 0.8 | 0.4 |
| | 6 | Main | 93.7 | 93.6 | 96.4 | 95.5 | 95.8 | 96.2 | 96.0 | NT | NT |
| | | ⁺Pk91 | 5.6 | 5.7 | 3.2 | 4.1 | 3.9 | 3.6 | 3.7 | NT | NT |
| | | ⁺Pk87 | 0.6 | 0.7 | 0.4 | 0.4 | 0.4 | 0.2 | 0.3 | NT | NT |

| | | | | | | | | | | |
|---|---|---|---|---|---|---|---|---|---|---|
| RP-HPLC | 0 | 92.6 | 92.3 | 92.5 | 92.4 | 92.5 | 93.1 | 93.2 | 93.1 | 92.3 |
| (% Main) | 1 | 93.0 | 93.5 | 93.5 | 93.4 | 93.2 | 92.8 | 91.7 | 92.4 | 88.5 |
| | 3 | 93.0 | 93.1 | 93.5 | 93.6 | 94.5 | 91.6 | 89.8 | 94.1 | 81.8 |
| | 6 | 89.6 | 89.8 | 91.5 | 89.0 | 90.3 | 88.9 | 84.5 | NT | NT |
| cIEF | 0 | 73.4 | 72.3 | 73.9 | 71.8 | 73.6 | 72.0 | 73.6 | 74.1 | 73.2 |
| (% Main) | 1 | 72.3 | 71.5 | 72.7 | 70.8 | 71.1 | 72.5 | 71.4 | 73.5 | 71.1 |
| | 3 | 67.6 | 68.4 | 69.9 | 69.5 | 69.9 | 65.7 | 52.8 | 68.1 | 56.1 |
| | 6 | 64.1 | 63.9 | 67.2 | 65.4 | 63.8 | 56.3 | 45.2 | NT | NT |
| CGE | 0 | 96.7 | 96.3 | 96.6 | 96.3 | 96.5 | 96.7 | 96.6 | 96.4 | 97.8 |
| (Non-reduced | 1 | 96.0 | 95.7 | 96.7 | 96.7 | 96.8 | 96.7 | 96.9 | 95.6 | 94.3 |
| % Main) | 3 | 94.5 | 94.2 | 95.8 | 95.5 | 95.3 | 95.7 | 95.4 | 93.6 | 90.8 |
| | 6 | 91.7 | 90.9 | 93.7 | 93.1 | 93.6 | 94.1 | 94.1 | NT | NT |
| CGE | 0 | 99.0 | 98.9 | 98.9 | 98.9 | 98.9 | 99.0 | 99.0 | 99.1 | 99.3 |
| (Reduced | 1 | 99.3 | 99.2 | 99.4 | 99.4 | 99.4 | 99.5 | 99.5 | 99.2 | 99.2 |
| % Main) | 3 | 98.8 | 98.5 | 98.9 | 98.8 | 98.8 | 99.0 | 98.9 | 98.5 | 95.9 |
| | 6 | 97.2 | 97.3 | 98.2 | 97.3 | 97.7 | 97.6 | 97.7 | NT | NT |
| Osmolality | 0 | 355 | 339 | 332 | 342 | 356 | 365 | 357 | 378 | 369 |
| (mOsm/kg) | 1 | 361 | 347 | 335 | 341 | 353 | 363 | 354 | 377 | 367 |
| | 3 | 347 | 346 | 336 | 350 | 350 | 361 | 362 | 380 | 373 |
| | 6 | 356 | 352 | 332 | 346 | 362 | 361 | 356 | NT | NT |
| *Bioassay | 0 | 115.7 | 104.6 | NT | NT | NT | NT | NT | NT | NT |
| (% Relative | 1 | NT | NT | NT | NT | NT | NT | NT | NT | NT |
| potency) | 3 | 119.6 | 97.2 | 112.0 | 115.2 | 113.9 | 137.7 | 154.8 | NT | NT |
| | 6 | 182.4 | 165.5 | 160.3 | 173.5 | 196.3 | 199.8 | 181.4 | NT | NT |

¹GA: clear, yellow (between Y1 and Y2 but more yellow [less brown]), essentially particle free solution
²GA: clear, yellow (between Y1 and Y2), essentially particle free solution
³GA: clear, yellow (Y2 or Y2 but slightly more yellow) essentially particle free solution
⁵GA: clear, yellow (Y1), essentially particle free solution
⁶GA: clear, yellow (slightly darker than Y1), essentially particle free solution
⁷GA: clear, yellow (darker than Y1), essentially particle free solution
⁸GA: clear, yellow (Y1 but slightly more yellow, less brown) essentially particle free solution
⁹GA: clear, yellow (darker and more yellow [less brown]than Y1) essentially particle free solution
¹⁰GA: clear, yellow (between CY2 and CY3), essentially particle-free solution
NT: Not Tested

TABLE 57
Stability Data at 40° C.

| | Time | | Formulation | | | | | | | | |
|---|---|---|---|---|---|---|---|---|---|---|---|
| Assay | (Month) | | 1 | 2 | 3 | 4 | 5 | 6 | 7 | 10 | 11 |
| General | 0 | | ¹GA | ²GA | ²GA | ²GA | ³GA | ³GA | ³GA | ⁶GA | ⁷GA |
| appearance | 1 | | ³GA | ⁴GA | ²GA | ²GA | ²GA | ²GA | ³GA | ⁸GA | ⁹GA |
| PH | 0 | | 7.1 | 7.1 | 5.9 | 6.4 | 6.4 | 7.1 | 6.4 | 7.2 | 7.2 |
| | 1 | | 7.0 | 7.0 | 5.9 | 6.4 | 6.4 | 7.1 | 6.5 | 7.0 | 6.9 |
| Concentration | 0 | | 79.2 | 80.8 | 79.8 | 80.0 | 78.6 | 79.2 | 79.5 | 118.7 | 123.0 |
| (RP-HPLC | 1 | | 79.0 | 79.9 | 78.7 | 78.7 | 77.9 | 78.6 | 79.1 | 118.3 | 122.2 |
| mg/mL) | | | | | | | | | | | |
| SEC-HPLC | 0 | main | 98.0 | 97.8 | 97.9 | 97.8 | 97.8 | 98.0 | 98.0 | 97.9 | 98.7 |
| (%) | | ⁺Pk91 | 1.8 | 2.0 | 1.9 | 1.9 | 1.9 | 1.8 | 1.7 | 1.9 | 1.6 |
| | | ⁺Pk87 | 0.2 | 0.2 | 0.3 | 0.3 | 0.2 | 0.2 | 0.2 | 0.2 | 0.1 |
| | 1 | Main | 82.2 | 82.4 | 95.7 | 94.7 | 94.9 | 96.3 | 96.2 | 80.2 | 77.7 |
| | | ⁺Pk91 | 11.9 | 11.5 | 3.3 | 4.4 | 4.2 | 3.4 | 3.3 | 10.7 | 5.5 |
| | | ⁺Pk87 | 2.9 | 2.9 | 0.7 | 0.7 | 0.6 | 0.3 | 0.5 | 3.2 | 1.8 |
| | | ⁺Larger | 3.0 | 3.2 | 0.3 | 0.2 | 0.2 | 0 | 0 | 6.0 | 14.6 |
| | | Frag1 | 0 | 0 | 0 | 0 | 0 | 0 | 0 | 0 | 0.4 |
| | | Frag2 | 0 | 0 | 0 | 0 | 0 | 0 | 0 | 0 | 0.1 |
| | 2 | Main | NT | NT | 94.7 | 93.2 | 93.2 | 95.4 | 94.9 | NT | NT |
| | | ⁺Pk91 | NT | NT | 3.7 | 5.2 | 5.5 | 4.3 | 4.5 | NT | NT |
| | | ⁺Pk87 | NT | NT | 0.7 | 1.0 | 0.7 | 0.3 | 0.6 | NT | NT |
| | | ⁺Larger | NT | NT | 0.9 | 0.6 | 0.6 | 0 | 0 | NT | NT |

| | | | | | | | | | | |
|---|---|---|---|---|---|---|---|---|---|---|
| RP-HPLC | 0 | 92.6 | 92.3 | 92.5 | 92.4 | 92.5 | 93.1 | 93.2 | 93.1 | 92.3 |
| (% Main) | 1 | 86.8 | 87.6 | 91.0 | 89.8 | 90.9 | 89.9 | 86.1 | 86.9 | 81.0 |
| cIEF | 0 | 73.4 | 72.3 | 73.9 | 71.8 | 73.6 | 72.0 | 73.6 | 74.1 | 73.2 |
| (% Main) | 1 | 61.5 | 62.0 | 65.6 | 62.6 | 63.5 | 61.7 | 58.5 | 61.5 | 54.8 |
| | 2 | 56.4 | 51.3 | 58.8 | 58.0 | 64.0 | 50.6 | 44.2 | NT | NT |
| CGE | 0 | 96.7 | 96.3 | 96.6 | 96.3 | 96.5 | 96.7 | 96.6 | 96.4 | 97.8 |
| (Non-reduced | 1 | 78.2 | 78.1 | 94.7 | 94.5 | 94.8 | 95.8 | 95.1 | 81.5 | 85.3 |
| % Main) | | | | | | | | | | |
| CGE | 0 | 99.0 | 98.9 | 98.9 | 98.9 | 98.9 | 99.0 | 99.0 | 99.1 | 99.3 |
| (Reduced | 1 | 98.0 | 97.9 | 98.8 | 98.6 | 98.6 | 98.9 | 98.9 | 97.7 | 95.8 |
| % Main) | | | | | | | | | | |
| Osmolality | 0 | 355 | 339 | 332 | 342 | 356 | 365 | 357 | 378 | 369 |
| (mOsm/kg) | 1 | 356 | 345 | 336 | 346 | 358 | 367 | 362 | 386 | 380 |
| *Bioassay | 0 | 115.7 | 104.6 | NT | NT | NT | NT | NT | NT | NT |
| (% Relative | 1 | NT | NT | 167.1 | 153.8 | 170.9 | 194.1 | 183.9 | NT | NT |
| potency) | | | | | | | | | | |

¹GA: clear, yellow (between Y1 and Y2 but more yellow [less brown]), essentially particle free solution
²GA: clear, yellow (between Y1 and Y2), essentially particle free solution
³GA: clear, yellow (Y2 or Y2 but slightly more yellow) essentially particle free solution
⁴GA: clear, yellow (Y2 but slightly more yellow) solution with one large grey sliver looking particle which quickly settles upon swirling
⁶GA: clear, yellow (slightly darker than Y1), essentially particle free solution
⁷GA: clear, yellow (darker than Y1), essentially particle free solution
⁸GA: clear, yellow (darker and more yellow [less brown]than Y1) essentially particle free solution
⁹GA: clear, yellow (CY2), essentially particle free solution
NT: Not Tested

TABLE 58
Degradation Rate and Slope P-value for each Assay of Formulations

| | | Formulation | | | | | | | | | | | | | |
|---|---|---|---|---|---|---|---|---|---|---|---|---|---|---|---|
| | | 1 | | 2 | | 3 | | 4 | | 5 | | 6 | | 7 | |
| | | Slope | p- | Slope | p- | Slope | p- | Slope | p- | Slope | p- | Slope | p- | Slope | p- |
| Assay | Temperature | (%/wk) | value | (%/wk) | value | (%/wk) | value | (%/wk) | value | (%/wk) | value | (%/wk) | value | (%/wk) | value |
| SEC- | 2-8° C. | −0.033 | 0.03 | −0.017 | 0.07 | 0 | 0.54 | 0 | 0.92 | 0 | 0.41 | 0 | 0.14 | 0 | 0.41 |
| HPLC | 25° C. | −0.15 | 0.04 | −0.15 | 0.05 | −0.05 | 0.03 | −0.08 | 0.02 | −0.07 | 0.04 | −0.07 | 0.03 | −0.07 | 0.02 |
| RP- | 2-8° C. | 0 | 0.65 | 0 | 0.23 | 0 | 0.17 | 0 | 0.47 | 0 | 0.22 | 0 | 0.65 | 0 | 0.79 |
| HPLC | 25° C. | 0 | 0.14 | 0 | 0.21 | 0 | 0.38 | 0 | 0.22 | 0 | 0.41 | −0.17 | 0.007 | −0.33 | 0.004 |
| cIEF | 2-8° C. | 0 | 0.25 | 0 | 0.66 | 0 | 0.41 | 0 | 0.21 | 0 | 0.36 | 0 | 0.83 | −0.066 | 0.03 |
| | 25° C. | −0.37 | 0.03 | −0.33 | 0.002 | −0.26 | 0.01 | −0.24 | 0.007 | −0.36 | 0.02 | −0.65 | 0.01 | −1.16 | 0.05 |
| CGE | 2-8° C. | 0 | 0.23 | 0 | 0.92 | 0 | 0.65 | 0 | 0.92 | 0 | 0.82 | 0 | 0.82 | 0 | 0.48 |
| NR | 25° C. | −0.19 | <0.001 | −0.21 | 0.003 | −0.12 | 0.02 | −0.14 | 0.04 | −0.12 | 0.03 | −0.11 | 0.01 | −0.11 | 0.03 |
| CGE | 2-8° C. | 0 | 0.32 | 0 | 0.93 | 0 | 0.45 | 0 | 0.89 | 0 | 0.64 | 0 | 0.62 | 0 | 0.68 |
| R | 25° C. | −0.076 | 0.07 | −0.069 | 0.05 | 0 | 0.17 | −0.071 | 0.10 | 0 | 0.11 | 0 | 0.11 | −0.060 | 0.10 |

### Effect of pH on Aggregation of Albiglutide During Diafiltration

The effect of pH on aggregation of albiglutide during diafiltration was also investigated. Samples were prepared by UFDF in DPD at pH 5.0, 5.7, and 6.5. After UFDF, the samples were tested by pH and SEC-HPLC. The results are shown in Table 59.

TABLE 59
SEC-HPLC and pH Results of albiglutide Diafiltration at Different pH

| Diafiltration | | SEC-HPLC results | |
|---|---|---|---|
| target pH | pH measured | % Monomer | Pk91% |
| 5.0 | 4.92 | 99.1 | 0.9 |
| 5.7 | 6.10 | 98.9 | 1.1 |
| 6.5 | 6.41 | 99.2 | 0.8 |

### DISCUSSION

### Stability of Albiglutide P3 Material in Different Formulations

Four samples were originally obtained from DPD. They were P3 and P3 heat-treated material in 10 mM phosphate, 153 mM mannitol, 117 mM trehalose, 0.01% PS80 at pH 7.2 (current lyophile formulation) and 5 mM citrate, 100 mM arginine, 117 mM trehalose, ˜0.01% PS80 (optimized formulation). P3 and P3 heat-treated materials in the optimized formulation had higher amount of aggregates and lower osmolality (123 and 74 mOsm/kg) and the citrate, arginine peak in SEC-HPLC was not seen.

Stability of P3 and P3 heat-treated materials in different formulations (F1 to F7) was discussed in this section. DSC results from Table 54 indicate that the thermal stability of heat-treated albiglutide is slightly more stable than non-heat-treated material as shown by Tm1, but not by Tonset (F1 vs F2). Tonset and Tm1 of F3, which is pH 5.7, 5 mM citrate, 100 mM arginine hydrochloride, and 117 mM trehalose (optimized solution formulation), is much higher than that of formulations F1, F2, F4 and F5, indicating albiglutide is more thermally stable in optimized solution formulation. Higher ΔH1 of F3 than that of formulations F1, F2, F4 and F5 also indicate F3 is more stable. Tm1 and Tm2 of albiglutide with (F5) and without (F4) Met in pH 6.5, 10 mM phosphate, 100 mM arginine hydrochloride, and 117 mM trehalose are similar indicating that Met does not affect the thermal stability of albiglutide. Addition of sodium octanoate to the solution formulation of albiglutide (F6 and F7) makes the Tonset, Tm1 and Tm2 much higher, and the first transition very close to the second transition peak indicating octanoate can thermally stabilize albiglutide in solution. The shift of the transition peak is probably due to the binding of octanoate to rHSA. The increased ΔH1 of F6 and F7 also indicate the addition of octanoate increased the thermal stability of albiglutide.

### Stability of F1 to F7 at 2-8° C.

The stability data of F1 and F2 at 2-8° C. (Table 55) indicate there is no difference between heat-treated and non-heat-treated albiglutide as measured by all assays. Albiglutide is less stable in solution in the current lyophile formulation (F1 and F2) than optimized solution formulation and the formulations with sodium octanoate. The decrease of % monomer of SEC-HPLC from 0 to 6 or 12 months at 2-8° C. is about 0.9% for F1 and F2, only 0.2-0.3% for other formulations. The decrease of % monomer corresponds to the increase of peak 91, which is the dimer peak.

After storage at 2-8° C. for 12 months, there is no difference among all the formulations as measured by general appearance, pH, concentration, RP-HPLC, cIEF, CGE (non-reduced and reduced), Osmolality and Bioassay, except the CGE results (R and NR) of formulation 4 at 2-8° C. for 6 months is lower and the Bioassay of formulation 4 at 2-8° C. for 6 months is higher. The sample of formulation 4 at 2-8° C. for 6 months was contaminated. Some microorganisms were observed after the sample was stored at 2-8° C. for over 7 days. The contamination most likely caused the clipping of albiglutide and resulted in the decrease of CGE % main, the presence of a fragment peak (EE560621) and increase of Bioassay.

The degradation rate of all formulations at 2-8° C. was not statistically significant (see Table 54), except F1 and F2 (current lyophile formulation) measured by SEC-HPLC and F7 (with sodium octanoate at pH 6.5) measured by cIEF. Albiglutide in optimized solution formulation, in formulation of pH 6.5, 10 mM phosphate, 100 mM arginine hydrochloride, and 117 mM trehalose with and without methionine, and in current lyophile formulation with 10 mM sodium octanoate is stable at 2-8° C. for at least 12 months.

### Stability of F1 to F7 at 25° C./60% RH

The stability data of albiglutide at 25° C. (Table 56) indicate there is no difference between heat-treated and non-heat-treated albiglutide as measured by all assays (F1 versus F2). The results for all formulations are similar as measured by general appearance, pH, concentration and osmolality. SEC-HPLC results as shown in Table 56 indicate albiglutide in optimized solution formulation (F3) is most stable. F3 has the highest % monomer peak and lowest % aggregate, after 6 months. Formulations with sodium octanoate (F6 and F7) are slightly more stable than formulations in pH 6.5, 10 mM phosphate with and without Met (F5 and F4). Albiglutide in solution in the current lyophile formulation (F1 and F2) showed the highest aggregation.

Results of CGE non-reduced as shown in Table 56, indicate all formulations have similar stability, except F1 and F2, which have lower % main. Results of CGE reduced as shown in Table 56, show F3 is most stable. F5, F6, and F7 (with Met (F5), sodium octanoate (F6 and F7)) are slightly more stable than F1, F2 and F4.

RP-HPLC and cIEF results as shown in Table 56, also indicate albiglutide is more stable in optimized solution formulation. Formulations with sodium octanoate have the lowest amount of % main and are less stable than other formulations. cIEF results of formulations with octanoate (F6 and F7) showed appearance of peak 91 and increase of peak 97 (23.1% for formulation 6 and 26.3% for formulation 7 compared to 14.2% for formulation 3). This result is probably due to the oxidation of methionine, cysteine, and tryptophan. This result is also supported by albiglutide Product-Related Variant Characterization Report, which indicated that the increase of peak 97 is due to N-terminal and lysine carbamylation, cysteine and tryptophan oxidation. Albiglutide in solution in the current lyophile formulation is similar to that in pH 6.5, 10 mM phosphate formulation. Formulations with and without Met have similar RP-HPLC and cIEF results, indicating that methionine did not inhibit chemical degradation of albiglutide at 25° C. for 6 months. All samples at 25° C. for 3 months pass the acceptance criteria for Bioassay; however, all samples at 25° C. for 6 months did not meet the Bioassay acceptance criteria.

Statistical analysis of the 25° C. stability data (Table 58) shows that albiglutide had statistically significant degradation measured by SEC, CGE NR, and cIEF in all formulations tested. Albiglutide in optimized solution formulation (F3) at 25° C. has the lowest degradation rate as measured by SEC-HPLC. Formulations F3, F4, F5, F6, and F7 all had comparable degradation rates measured by CGE NR. Albiglutide has the lowest degradation rates of cIEF % main in F3 and F4 (0.26 and 0.24%/week, respectively). F6 and F7 (containing octanoate) had the highest rates of cIEF % main degradation (0.65 and 1.17%/week, respectively). F6 and F7 (with sodium octanoate) were the only formulations that showed statistically significant degradation by RP-HPLC.

Comparison of the degradation rates of F4 (without Met) and F5 (with Met) shows that addition of methionine to the pH 6.5 formulation increases the rate of degradation measured by cIEF % main from 0.24%/week to 0.36%/week at 25° C., but does not impact degradation rates of any other assays. Overall, the stability data at 25° C. indicate that F3 (optimized formulation) is the most stable formulation for albiglutide.

### Stability of F1 to F7 at 40° C./75% RH

The results at 40° C. as shown in Table 57 indicate the stability of non-heat-treated material is similar to that of heat-treated material (F1 versus F2). Results from SEC-HPLC as shown in Table 57 indicate albiglutide in F6 (with sodium octanoate in pH 7.2) is most stable, with highest percent of monomer and lowest percent of aggregation. Albiglutide in F3 (optimized solution formulation) is second most stable and similar to F7 (with sodium octanoate in pH 6.5, 10 mM phosphate, 100 mM arginine hydrochloride, and 117 mM trehalose). Addition of methionine did not affect aggregation (F4 versus F5). Albiglutide had the highest rate of aggregation in the current lyophile formulation (F1 and F2).

Results of CGE non-reduced are similar to the results of SEC-HPLC. Albiglutide in F6 is most stable, with % main of 95.8% after 1 month at 40° C. F2, F3, F4, and F7 had similar % main ranging from 94.5 to 95.1% after 1 month at 40° C. Albiglutide in solution in the current lyophile formulation is least stable, with 78.2 and 78.1% main after 1 month at 40° C. for F1 and F2, respectively.

Results of RP-HPLC as shown in Table 57 indicate F3 (optimized solution formulation) is similar to that of F5 (with Met), which are the most stable formulations, with the highest % main and least changes from 0 to 6 months. F7 containing sodium octanoate had the lowest % main. When comparing F4 (with Met) and F5 (without Met), albiglutide is more stable in the presence of Met, as evidenced by the decrease of % main from 0 to 1 month of 2.6% for F4, and only 1.6% for F5.

cIEF results as shown in Table 57 indicate F5 (with Met) is the most stable formulation. F3 is similar to F4 and F1 and is the second most stable formulation. F7 (octanoate, pH 6.5) is the least stable formulation in line with RP-HPLC results. Results of cIEF also indicate albiglutide is more stable in formulation with Met. The decrease of % main of cIEF from 0 to 6 months is 13.8% for F4 (no Met), and only 9.6% for F5 (with Met).

### Stability of Process 3 and Process 2 Material Comparison (F10 Versus F11)

Process 3 material (F10) is more stable than Process 2 material (F11) as shown by cIEF and CGE NR results after storage at 2-8° C. for 3 months (Table 55). The % main of cIEF decreased from 74.1 to 73.2 for F10, from 73.2 to 68.0 for F11. The % main of non-reduced CGE did not decrease for F10, and decreased 1.5% for F11. Process 3 material (F10) is more stable than process 2 material (F11) as shown by RP-HPLC, cIEF, and CGE (Reduced and non-reduced) results after storage at 25° C. for 3 months (Table 56). The % main of RP-HPLC did not decrease for F10, and decreased 10.5% for F11. The % main of cIEF decreased from 74.1 to 68.1 for F10, and from 73.2 to 56.1 for F11. The % main of non-reduced CGE decreased 2.8% for F10, decreased 7% for F11. The % main of reduced CGE decreased from 99.1 to 98.5 for F10, decreased from 99.3 to 95.9 for F11.

After storage at 40° C. for 1 month, process 3 material (F10) is more stable than process 2 material (F11) as shown by SEC-HPLC, RP-HPLC, cIEF, and CGE (Reduced) results (Table 57). The % monomer of SEC-HPLC decrease 17.7% for F10, decreased 21.0% for F11. The decrease in % monomer corresponds to an increase of higher order aggregates (peak larger than pK87). For F11, fragments were also observed. The % main of RP-HPLC decreased 6.2% for F10, and decreased 11.3% for F11. The % main of cIEF decreased 12.6% for F10, and 18.4% for F11. The % main of reduced CGE decreased 1.4% for F10, and decreased 3.5% for F11.

### Effect of pH on Aggregation of Albiglutide During Diafiltration

Diafiltration of albiglutide in pH 5.0, 5.7 and 6.5 in optimized solution formulation was performed in DPD. The % monomer for all samples is similar (Table 59). There is no difference among samples diafiltration in different pH in optimized solution formulation.

### Conclusion

Overall, the results indicate there is no difference between heat-treated and non-heat-treated materials. All formulations tested had equivalent stability at 2-8° C. for 12 months, except the current formulation (F1 and F2). Therefore, albiglutide in the optimized solution formulation at pH 5.7, optimized formulation at pH 6.5 with and without 10 mM methionine and 10 mM sodium octanoate, and in current formulation with 10 mM sodium octanoate is stable at 2-8° C. for at least 12 months. Stability data at 25° C. and 40° C. shows that albiglutide is most stable in the optimized formulation (pH 5.7, 5 mM citrate, 100 mM arginine hydrochloride, and 117 mM trehalose). Addition of Met can stabilize albiglutide at 40° C.; however, there was no significant effect at 2-8 and 25° C. Similar to process II material, addition of sodium octanoate to albiglutide Process III material decreases aggregation; however, it increased degradation measured by RP-HPLC and cIEF, particularly at pH 6.5. Process 3 material is more stable than Process 2 material as indicated by cIEF, RP-HPLC, SEC-HPLC and CGE results. Diafiltration of albiglutide in optimized solution formulation at pH 5.0, 5.7 and 6.5 does not cause aggregation.

### Example 3

A lead formulation was identified which is stable for 12 months at 2° C. to 8° C. and contains 5 mM citrate, 117 mM trehalose, 100 mM arginine, ˜0.01% Polysorbate 80, pH 5.9. Two additional formulation development studies were conducted seeking a liquid formulation providing improved stability for albiglutide over the lead formulation when stored at 2-8° C. in a pre-filled syringe (PFS).

### EXPERIMENTAL DESIGN

### Pre-Filled Syringe (PFS) Study

The formulations tested in the PFS Study are listed in Table 60. All formulations contained 117 mM trehalose, 100 mM arginine, ˜0.01% (w/w) PS80, at pH 6.0. The albiglutide concentration was approximately 100 mg/mL in all formulations.

TABLE 60
Formulations (all contain 117 mM trehalose, 100 mM arginine, 0.01% (w/w)
PS80, pH 6.0)

| Formulation | Buffer | Octanoate | Methionine | Tryptophan | Cysteine | NaCl | Prep |
|---|---|---|---|---|---|---|---|
| 1 | 5 mM | | | | | | Dialyze |
| | Citrate | | | | | | |
| 2 | 10 mM | | | | | | Spike |
| | Citrate | | | | | | from #1 |
| 3 | 5 mM | | | | | 50 | Spike |
| | Citrate | | | | | mM | from #1 |
| 4 | 5 mM | 10 mM | | | | | Spike |
| | Citrate | | | | | | from #1 |
| 5 | 5 mM | | 10 mM | | | | Spike |
| | Citrate | | | | | | from #1 |
| 7 | 5 mM | | 10 mM | 1 mM | | | Spike |
| | Citrate | | | | | | from #1 |
| 8 | 5 mM | | | | 10 mM | | Spike |
| | Citrate | | | | | | from #1 |

All of the formulations were filled into high silicone pre-filled syringes (PFS) with a volume of 0.75 ml. Formulation 1 was additionally filled into a vial (control). Each formulation and container combination is listed in Table 61 as a variation. The stresses evaluated (thermal, light, shear) per variation are also provided in Table 61.

TABLE 61
Variations and stresses evaluated

| | | | Thermal | Light Stress | Shear Stress |
|---|---|---|---|---|---|
| Variation | Formulation | Container | Stability Study | Study | Study |
| A | 1 | Vial | x | | x |
| B | 1 | PFS high silicone | x | x | x |
| E | 2 | PFS high silicone | x | x | |
| F | 3 | PFS high silicone | x | x | |
| G | 4 | PFS high silicone | x | x | x |
| H | 5 | PFS high silicone | x | x | |
| J | 7 | PFS high silicone | x | x | |
| K | 8 | PFS high silicone | x | x | |

### Aggregation Study

The formulation design for the Aggregation Study was based on the results of dynamic light scattering (DLS), Light Cycler (extrinsic fluorescence) and DSC experiments. Results of DSC, thermal unfolding by DLS, and extrinsic fluorescence showed elevated thermal stability of albiglutide with increasing NaCl, increasing trehalose, and decreasing arginine concentrations and the addition of cysteine. Sodium octanoate demonstrated protection against aggregation in previous studies. However, octanoate-containing formulations were observed in previous studies to have higher rates of chemical degradation. Formulations with sodium octanoate at different pH values were evaluated to determine if the increase in chemical degradation rate and decrease in aggregation rate in octanoate-containing formulations is pH dependent. A formulation with both methionine and octanoate was also evaluated to see whether methionine could prevent the chemical degradation induced by octanoate. The formulations tested in this study are listed in Table 62.

TABLE 62
Formulations (all approximately 100 mg/mL, contain 0.01% (w/w) PS80)

| | | Octa- | | | | | Tre- | |
|---|---|---|---|---|---|---|---|---|
| Formulation | Buffer | noate | Met | Arg | Cys | NaCl | halose | pH |
| High trehalose with | 10 mM Citrate | | | | | 50 mM | 200 mM | 6.0 |
| NaCl | | | | | | | | |
| High NaCl | 10 mM Citrate | | | | | 140 mM | | 6.0 |
| Arginine with NaCl | 10 mM Citrate | | | 100 mM | | 140 mM | | 6.0 |
| Trehalose with NaCl | 10 mM Citrate | | | | | 140 mM | 100 mM | 6.0 |
| Octanoate Met and NaCl | 10 mM Citrate | 10 mM | 20 mM | | | 140 mM | | 6.0 |
| NaCl and octanoate | 10 mM Citrate | 10 mM | | | | 140 mM | | 6.0 |
| NaCl and octanoate | 10 mM Phosphate | 10 mM | | | | 140 mM | | 7.0 |
| NaCl + cysteine | 10 mM Citrate | | | | 1.36 mM | 140 mM | | 6.0 |
| NaCl, octanoate and | 10 mM Phosphate | 10 mM | | | 1.36 mM | 140 mM | | 7.0 |
| cysteine | | | | | | | | |

### Sample Preparation:

### PFS Study

Four-hundred (400) mL of 103 mg/mL albiglutide in 10 mM sodium phosphate, 117 mM trehalose, 153 mM mannitol, ˜0.01% PS80, pH 7.0 was concentrated to ˜140 mg/mL and dialyzed into PFS Formulation 1 (Table 1). Dialysis was performed at 2-8° C. protected from light, 120 mL of albiglutide against 2 L buffer with 4 buffer changes, for a total of 8 L. Formulations 2-8 were prepared from Formulation 1 by spiking excipient stocks. Each formulation was adjusted to pH 6.0 using 1 M HCl and filtered through a 0.22 micron PES filter. Protein concentration of the resulting formulations was measured by RPHPLC concentration. All formulations were diluted to 100 mg/mL by addition of matching diluents. The concentration for Formulation 1 was ˜105 mg/mL, and 97.6-99.1 for other formulations. This ˜7% difference in protein concentration is not expected to impact the results of this study. Syringes were filled with 0.75 mL of bulk drug product solution and stoppered. For light stressed samples, syringes were placed horizontally in the variable intensity photostability chamber at 1000 lux, 25° C./65% RH, for 1 week. Only variations in the high silicone syringe were tested for light stress to compare formulations' ability to protect from light (Variations BEFGHJK). Dark controls were protected from light by wrapping with aluminum foil. Shear stress was induced by placing syringes horizontally on an orbital shaker at 25° C., protected from light, for 48 h at 300 rpm. Only the control and sodium octanoate formulations (Variations A, B, and G) were evaluated by shaking to assess the impact of silicone content and sodium octanoate on shear-induced degradation.

### Aggregation Study

Albiglutide in 5 mM citrate, 117 mM trehalose, 100 mM arginine, and ˜0.01% PS80, pH 6.0 was dialyzed into 10 mM citrate, pH 6.0 buffer (to prepare Aggregation Formulations 1 to 6 and 8) and 10 mM sodium phosphate, 140 mM NaCl, pH 7.0 buffer (to prepare Formulations 7 and 9). Dialysis was performed at 2-8° C. protected from light, 90 mL of albiglutide against about 3.5 L buffer with 3 buffer changes, for a total of 10 L. The protein concentration of the sample was measured by Solo VPE and osmolality was measured to confirm the dialysis was complete. Then the target formulations in Table 3 were prepared by spiking stock solutions of NaCl, trehalose, arginine or cysteine in 10 mM citrate, 0.01% PS80 at pH 6.0 or in 10 mM sodium phosphate, 140 mM NaCl, 0.01% PS80 at pH 7.0. pH was checked for each formulation and 1M NaOH was used to adjust pH to 6.0 for Formulation 1 to 4, and 8, adjusted to 7.0 for Formulation 7 and 9. Each sample was filtered through 0.2 um filter. Each formulation was filled to syringes with 0.75 mL for all assays except bioassay, which was filled with 0.25 mL for each syringe. The head space for the syringes was 2-3 mm. The samples were stored either at 2-8° C. or 30° C. per Table 64.

### Testing

TABLE 63
Testing schedule for thermal stress for PFS Study

| Temperature | Time 0 | 2 W | 1 M | 2 M | 3 M | 6 M |
|---|---|---|---|---|---|---|
| 2-8° C. | √ | | | | √ | √ |
| 30° C./65% RH | √ | | √ | √ | √ | |
| 40° C./75% RH | √ | √ | √ | √ | | |

TABLE 64
Testing schedule for Aggregation Study

| Temperature | Time 0 | 2 W | 1 M | 2 M | 3 M | 6 M | 9 M | 12 M | extra |
|---|---|---|---|---|---|---|---|---|---|
| 2-8° C. | √ | | √ | √ | √ | √ | √ | √ | 3 |
| | MFI | | | MFI | | | | MFI | |
| | Bioassay | | | Bioassay | | | | Bioassay | |
| | FcRn* | | | FcRn* | | | | FcRn* | |
| 30° C./65% | √ | √ | √ | √ | √ | | | | 2 |
| RH | | | | MFI | | | | | |
| | | | | Bioassay | | | | | |
| | | | | FcRn* | | | | | |

*Free Cys-94 in albiglutide may be involved in FcRn binding. Addition of Cys to the formulation may form disulfide bond with Cys-94 and affect FcRn binding. Formulation 8 and 9 (Cys containing formulation) together with BDS control were analyzed by FcRn binding.

### DISCUSSION

### PFS Study

Variation K was discontinued after 1 month due to the significant formation of peak 93 as measured by RP-HPLC. The following discussion will focus on Variation A to J for stability. Variations G and J underwent reduced testing after 1 months because Variation G demonstrated accelerated chemical degradation by RPHPLC and cIEF at 40° C., and Variation J (tryptophan/methionine) was showing no benefit compared to Variation H (methionine alone). Although the study ended at 6 months, Variation E was tested at 12 months to get a 12-month data point on the formulation chosen as the phase III/commercial albiglutide liquid formulation.

### Stability at 2-8° C.

Stability data indicate there is no change for Variation A, B, and F to J after storage at 2-8° C. up to 6 months, and Variation E after storage up to 12 months as measured by GA (general appearance), pH, concentration, RP-HPLC, cIEF, CGE reduced and non-reduced, osmolality and Bioassay. cIEF peak 102 split into 2 peaks at 6 months for all formulations, however, the split peak is less than DL.

Results of SEC-HPLC indicate the main peak slightly decreased from 99.0% to 98.2% for Variation A to J after storage at 2-8° C. up to 6 and 12 months, corresponding with the increase of the dimer peak from 1.0% to ˜1.6%. At 6 months, the variation of monomer across all formulations was within 0.1%. Peak 87 slightly increased from 0.1% to 0.2% from time 0 to 3 months; however, it did not increase further between 3 and 6 months. There is no significant difference in stability of albiglutide at 2-8° C. among different formulations and containers as measured by other assays, except Variation G has slightly lower % main (96.3% at 6 months) compare to time 0 data (97.0%) as measured by RP-HPLC.

MFI data indicate that the particle numbers in the glass vial is lower than that in syringes believed to be due to absence of silicon particles. The majority of total particles are particles less than 10 μm, about 30,000. Only about 300 particles larger than 10 μm are present. Total particle number does not increase with storage time at 2-8° C. For Variation G, total particle numbers slightly increased with storage time.

### Stability at 30° C.

Stability data indicated there is no significant change for Variation A to J after storage at 30° C. up to 3 months as measured by GA, pH, concentration, CGE reduced, and osmolality.

The purity of albiglutide in all formulations decreased with time, corresponding with the increase of the dimer and Peak 87 after storage at 30° C. up to 3 months as measured by SEC-HPLC. After storage at 30° C. for 3 months, the main peak decreased from ˜99.0% to 97.0%, the dimer peak increased from ˜1.0% to 2.4-2.6%, and peak 87, increased from 0.1% to 0.3-0.4%. Variation H (Met), had 0.1-0.3% higher of main peak than other variations.

Data indicate that the main peak of RP-HPLC of all variations decreased from ˜97% to ˜94% after storage at 30° C. for 3 months. Peak 96, which is 6AA impurity plus proteolytic fragments coeluted with 6AA impurity, increased from 2.2-2.3% to 3.1-3.6% and peak 93, which is glycosylated/glycated albiglutide, increased from 0.6-0.7% to 1.0-1.3%. Peak 90, which is also glycosylated/glycated albiglutide, increased from <DL to 0.4-0.6% (<QL). There is no significant difference among different variations.

cIEF results indicate that the percentage of the main peak decreased ˜10% for Variations A to F and H, acidic peak increased from ˜21% to ˜27%, and basic peak slightly increased from 4.0-4.9% to 5.7-6.4% after storage at 30° C. for 3 months. Variation G (octanoate) had significant lower main peak (69.0%) compared to other variations (74-76%) and was stopped for analysis. Variation J was also stopped for analysis due to no benefit compared to Variation H and the low solubility of tryptophan which would complicate manufacture.

CGE non-reduced results indicate that the main peak of albiglutide decreased after storage at 30° C. for 3 months for all formulations by approximately the same amount (˜3%). Bioassay results show the potency increased from ˜100% to ˜130% after storage at 30° C. for 3 months due to the release of potent peptide. There is no significant difference among all variations.

Overall, the results indicate that the stability of albiglutide decreased and the total particle numbers increased after storage at 30° C. up to 3 months as measured by SEC-HPLC, RP-HPLC, cIEF, CGE non-reduced and MFI. All variations demonstrated similar stability except Variation G containing sodium octanoate, which had lower stability as measured by cIEF.

### Stability at 40° C.

The stability of Variation A to J does not change significantly after storage at 40° C. up to 2 months as measured by GA, pH, concentration, CGE reduced, and osmolality.

SEC-HPLC results show the monomer peak decreased from ˜99% to 94-95%, corresponding with the increase of dimer from 1% to ˜3.5%, peak 87 from 0.1% to 0.4-0.7%, and peak 79 up to 1.9%. Peak 79, which is high order aggregates, was not observed in lyo formulations and is not in the specification of lyo drug product. At 40° C. for 2 months, Variation F (NaCl) and E (10 mM citrate) has higher percentage of monomer, 95.1% for F and 94.9% for E, indicating Variation F and E are more stable than other variations. Variation H (Met) has slightly higher percentage of monomer peak (94.5%) than Variations A and B, indicating Variation H is more stable then Variations A and B, but not as stable as Variations E and F.

RP-HPLC results indicate that the main peak decreased from ˜97% to ˜86% for all variations, except Variation H, which has higher main peak (88.3%). All minor peaks increased at a similar level except Variation H, peak 96, which is 6AA impurity plus proteolytic fragments coeluted with 6AA impurity, increased from ˜2.2% to 5.8%. Peak 93, which is glycosylated/glycated albiglutide, increased from 0.6% to ˜1.4%. Peak 90, which is also glycosylated/glycated albiglutide and peak 87, which is the N-terminal cleaved albiglutide proteolytic fragments, increased from <DL to ˜1.4% and 0.4% (<QL) respectively, Variation H (Met) has lower level increase for all minor peaks. RP-HPLC results indicate that Variation H is more stable. Variation G has significant lower main peak at 0.5 month (94.0%) and 1.0 month (90.4%) compare to other variations and was stopped for analysis after 1 month. Variation J was also stopped for analysis due to no benefit compared to Variation H.

cIEF results indicate the main peak decreased from 74-76% to 57-58%, acidic peak increased from ˜20% to 33-34% and basic peak increased from 4-5% to 7-9%. Variation G has lower stability and was stopped for analysis after 1 month.

Results indicate the percentage of main peak decreased ˜2-3% for all formulations after storage at 40° C. up to 2 months as measured by non-reduced CGE. The Bioassay data show the potency increased from ˜100% to ˜180% for Variations A and B after storage at 40° C. for 1 month due to the release of potent peptide. MFI data indicate the total particle number slightly increased about 1-2 times after storage at 40° C. for 2 months. For Variation G, particles numbers larger than 10 μm increased after storage at 40° C. for 1 month.

Overall, the results indicate that the stability of albiglutide decreased and the total particle numbers increased after storage at 40° C. up to 2 months as measured by SEC-HPLC, RP-HPLC, cIEF, CGE non-reduced and MFI. Variation E (10 mM citrate) and F (NaCl) are more stable as measured by SEC-HPLC. Variation H (Met) is more stable as measured by RP-HPLC, but less stable than E and F as measured by SEC-HPLC. Variation G (octanoate) has lower stability as measured by RP-HPLC and cIEF.

### Effect of Light

The appearance, pH and Bioassay of Variation A to K do not change significantly after exposure to 1000 lux light, 25° C./65% RH, for 7 days. The stability of Variation A to K decreased after exposed to light as measured by SEC-HPLC, RP-HPLC, cIEF, CGE (NR and R). For RP-HPLC, new peak 108 and peak 112 was shown in light exposed samples.

Variation K, which contains cysteine, had the highest stability after exposure to light as measured by SEC-HPLC, RP-HPLC, cIEF and CGE (NR and R). Variations H and J, which contain Met and Met/Try respectively, was slightly more stable than Variation B (5 mM citrate) as measured by SEC-HPLC and non-reduced CGE, and more stable than Variation E (10 mM citrate) and F (50 mM NaCl) as measured by SEC-HPLC, cIEF and CGE (NR and R). Variation B is slightly more stable than Variations E and F. Variation G, which is the formulation containing octanoate, is least stable under light stress as measured by SEC-HPLC and RP-HPLC.

SEC-HPLC data show the monomer peak decreased 4.5% for Variation K, decreased ˜6% for Variation H and J, decreased 7.4% for Variation B, decreased 8.5% for Variation E and F, and decreased 8.9% for Variation G. RP-HPLC results show the main peak decreased ˜0.6% for Variation K, decreased ˜6% for Variation H and J, decreased 4.9% for Variation B, decreased 5.7% for Variation E and F, and decreased 11.4% for Variation G. cIEF data show the main peak decreased 17.3% for Variation K, decreased ˜30% for Variation B, H and J, decreased 36-38% for Variation E and F, and G. CGE non-reduced data show the main peak decreased 1.0% for Variation K, decreased ˜7% for Variation H and J, decreased 8.4% for Variation B, and decreased 9.3-9.9% for Variation E, F and G. CGE reduced data show the main peak decreased 0.2% for Variation K, decreased ˜2.3% for Variation H and J, decreased 2.8% for Variation B, and decreased 3.2-3.4% for Variation E, F and G.

Effect of Stress under Shaking

The stability of Variations B and G does not change significantly after shaking at 300 rpm for 48 hours at room temperature protected from light as measured by GA, pH, SEC-HPLC, RP-HPLC, cIEF, CGE (NR and R) and Bioassay. Variation A, which is the lead formulation in the glass vial, has slightly lower percentage of main and higher percentage of dimer peak after shaking as measured by SEC-HPLC, and had lower main peak as measured by CGE NR. This is probably due to the higher head space in glass vial (about 2 cm) than that in the syringes (about 2-3 mm). Particle numbers indicate that particle numbers did not change significantly after shaking for all variations.

### Aggregation Study

The study was performed for samples at 2-8° C. up to 2 months and Formulations 1-6 at 30° C. up to 3 months. The study was stopped after 3 months due to no formulation demonstrating increased stability over lead formulation (control).

### Stability at 2-8° C.

Data indicate there is no significant stability change for all formulations and no significant difference among all formulations at 2-8° C. up to 2 months as measured by GA (general appearance), pH, concentration, CGE non-reduced and reduced, osmolality and Bioassay.

The results measured by SEC-HPLC indicate the stability of albiglutide slightly decreased after storage at 2-8° C. for 2 months for all formulations. Formulation 1 and lead formulation (control) are the most stable formulations and Formulations 2 (high NaCl), 6 (NaCl and octanoate at pH 6.0), and 7 (NaCl and octanoate at pH 7.0) are less stable than other formulations. SEC-HPLC monomer peak decreased 0.2% for Formulation 1 and lead formulation, decreased 0.4-0.5% for Formulation 2, 6 and 7, and decreased about 0.3-0.4% for other formulations.

The results measured by RP-HPLC show that the lead formulation is more stable than all other formulations, with no decrease of the main peak, and no increase of minor peaks. Formulation 8 and 9, which contain cysteine, are not stable at 2-8° C. The main peak decreased significantly at time 0 and 2-8° C. for 1 months (decreased 35% at time 0 for Formulation 9, decreased 14% at 1 month for Formulation 8), corresponding with significant increase in Peak 96 (increased 35% at time 0 for Formulation 9, increased 14% at 1 month for Formulation 8). Peak 96 is 6AA impurity plus proteolytic fragments coeluted with 6AA impurity according to the characterization report⁷. For Formulation 1 to 7, the main peak decreased from ˜97.2% to ˜96.7%.

Results of cIEF indicate that the lead formulation and Formulation 4 are slightly more stable than all other formulations, with no decrease of main peak. Formulations 7 and 9, which contain sodium octanoate at pH 6.0 and Formulation 8 (Cys) are less stable than other formulations. Main peak decreased 3.0% for Formulation 7, decreased 1.9% for Formulation 9 and decreased 1.1% for Formulation 8, and only decreased 0.1-0.8% for other formulations. Split of peak 102 was not observed for all samples.

Results of FcRn binding indicate that the formulation containing cysteine affects the binding of albiglutide to Fc receptor and will affect the half-life of albiglutide. FcRn binding decreased 6% for Formulation 8, and decreased 16% for Formulation 9 and no change for the lead formulation. Fluorescence data indicate the fluorescence intensity decreased for Formulation 8 (Cys), fluorescence intensity decreased and had a blue shift for Formulation 9 (Cys and octanoate) compared to the lead formulation after the samples storage at 2-8° C. for 2 months. Formulation 6 (octanoate) also had lower fluorescence intensity and blue shift compared to the lead formulation, all other formulations did not change after the samples storage at 2-8° C. for 2 months. MFI data indicate that the total particle numbers of Formulation 7 and 9, doubled after storage at 2-8° C. for 2 months. All other formulations do not change.

Overall, the results of data at 2-8° C. indicate that the lead formulation is more stable than other formulations as measured by RP-HPLC and cIEF. The lead formulation and Formulation 1 (high trehalose) are more stable as measured by SEC-HPLC.

### Stability at 30° C.

There is no significant stability change for all formulations and no significant difference among all formulations at 30° C. for 3 months as measured by GA (general appearance), pH, concentration, CGE reduced, and osmolality.

SEC-HPLC results indicate that the main peak decreased with time after storage at 30° C. for all formulations. Formulations 6, 7 and 9, which contain octanoate, are the least stable formulations with the monomer peak decrease from 99% to 96.2-96.7% and the dimer peak increased from 1.0% to 3.0-3.5%. For other formulations, monomer peak decreased from 99% to 97.0-97.6%, dimer peak increased from 1.0% to 2.1-2.7% after storage at 30° C. for 2 months. Formulation 7, 8 and 9 was stopped for analysis after 2 months, due to chemical degradation caused by Cys and octanoate as measured RP-HPLC and cIEF. Data at 30° C. for 3 months indicate that Formulation 3 (NaCl+Arg) and the lead formulation are more stable than Formulation 1, 2, 4 and 5. For Formulation 3 and the lead formulation, the monomer peak decreased from 98.9% to 97.3%, the dimer peak increased from 1.0% to 2.2%, for Formulation 1, 2, 4 and 5, the monomer peak decreased from 98.9% to 96.5-96.9%, the dimer peak increased from 1.0% to 2.4-2.7%.

RP-HPLC show that the main peak decreased and all minor peaks increased with time at 30° C. RP-HPLC main peak decreased from ˜97% to 89.9-94.0%. Peak 96, which is 6AA impurity plus proteolytic fragments coeluted with 6AA impurity, increased from ˜2% to 3.8-7.5%. Peak 93, which is glycosylated/glycated albiglutide, increased from 0.6-0.8% to 1.2-1.5%. Peak 90, which is also glycosylated/glycated albiglutide, increased from <DL to 0.6-0.9% and peak 87, which is the N-terminal cleaved albiglutide proteolytic fragments, increased from <DL to 0.4-0.6% (<QL) after storage at 30° C. for 3 months. Formulations 8 and 9, which contain cysteine, are not stable. The main peak decreased to 59.7% and 20.3% respectively, and peak 96, which is 6AA impurity plus proteolytic fragments coeluted with 6AA impurity, increased to 39.0% and 77.4% respectively after storage at 30° C. for 0.5 months. Formulation 7, 8 and 9 were stopped for analysis after 2 months. Data at 30° C. for 3 months indicate that the lead formulation, Formulation 2 (high NaCl) and 4 (NaCl+trehalose) are more stable than Formulations 1, 3, 5 and 6. The main peak decreased 2.9-3.1% and peak 96 increased 1.1-1.6% for the lead formulation, Formulations 2 and 4, while the main peak decreased 3.5-7.5%, peak 96 increased 1.9-5.5% for Formulations 1, 3, 5 and 6. Other minor peaks did not show significant difference.

Degradation as measured by cIEF was similar to results of RP-HPLC for formulations evaluated. The main peak decreased for all formulations. cIEF main peak decreased from 75.3-78.9% to 59.4-68.9%, total acidic peaks increased from 16.9-19.9% to 25.5-34.9%, and total basic peaks increased from 3.9-4.5% to 4.8-6.5%. Data at 30° C. for 2 months indicate that Formulations 5, 6, and 7, which have octanoate, and Formulation 8 and 9, which have Cys, are less stable than other formulations. cIEF main peak decreased ˜21% for Formulation 7, decreased ˜12% to ˜14% for Formulations 5, 6 and 9, decreased 9.3% for Formulation 8, and decreased ˜8.5% for Formulations 1 ˜4. Formulation 7, 8 and 9 were stopped for analysis after 2 months. Data at 30° C. for 3 months indicate that the lead formulation is most stable, Formulations 1 to 4 are more stable than Formulations 5 (octanoate+Met) and 6 (octanoate). cIEF main peak decreased 3.4% for the lead formulation, decreased 7.0-8.0% for Formulations 1-4, decreased 12.5% and 13.6% for Formulations 5 and 6.

CGE NR data indicate that the stability of albiglutide decreased with time, but the main peak increased at 3 months. This is probably due to different batches of the chip and reagent (different batch of chips were used for different time points). The results again show that Formulations 5, 6, 7, 8 and 9 are less stable than Formulations 1-4 and the lead formulation. The lead formulation is the most stable after storage at 30° C. for 2 months. The main peak of the lead formulation at 30° C. for 3 months decreased, but increased for all other formulations. The lead formulation and all other samples were measured at different times and with different CGE chips. The percentage of main peak should not increase after storage at 30° C. for 3 months, so it is impossible to compare the 30° C. 3 months data of the lead formulation against the other formulations.

Bioassay data show the potency increase from ˜100% to ˜130-160% for all formulations after storage at 30° C. for 2 months and there is no significant difference among different formulations. FcRn binding decreased 23% for both Formulation 8 and Formulation 9, and no change for the lead formulation after storage at 30° C. for 2 months. MFI indicate that the total particle numbers increased for Formulations 6 and 7, which contain octanoate, after storage at 30° C. for 2 months. All other formulations did not change.

Overall, the results indicate that Formulations 8 and 9, which contains Cys have some new species as measured by RP-HPLC. Formulations 5-9, which contain octanoate, Cys or both, are less stable as measured by RP-HPLC, cIEF and CGE non-reduced. The lead formulation and Formulation 3 are more stable than other formulations as measured by SEC-HPLC. The lead formulation, Formulations 2 and 4 are more stable than other formulations as measured by RP-HPLC. The lead formulation is the most stable formulation as measured by cIEF.

### CONCLUSION

Overall, the results indicate that the formulations containing octanoate (Variation G) in PFS Study reduce aggregation rates but are less stable than other formulations with respect to chemical degradation as indicated in both PFS Study and Aggregation Study. The Formulation containing cysteine (Variation K in PFS Study, Formulations 8 and 9 in Aggregation Study) is very likely to have new species as measured by RP-HPLC, even though cysteine significantly protects albiglutide from light-induced degradation. Formulation 8 (Cys) and Formulation 9 (Cys and octanoate) has lower FcRn binding after storage at 2-8° C. and 30° C. for 2 months. The binding of HSA to FcRn is pH-dependent. Three conserved histidine residues, His-464 in HSA subdomain DIIIa, His-535 in DIIIb and His-510 in the loop connecting the two, are crucial for the pH-dependent binding of HSA to FcRn. Free Cys94 oxidation was found to affect FcRn binding in the report of evaluation of the impact of albiglutide forced degradation of FcRn binding. In this study, tertiary structure change was found in Formulation 8 (Cys) and Formulation 9 (Cys and octanoate) as measured by fluorescence. The binding of Cys and octanoate to HSA probably caused the tertiary structure change and decrease the binding of albiglutide to FcRn.

Met and Trp have a partial reduction in light-induced degradation and a slight benefit by RP-HPLC at 40° C. compared to the 10 mM citrate formulation, but have no effect on stability at recommended storage condition of 2-8° C. in both studies.

The lead formulation, which is 5 mM citrate, 117 mM trehalose, 100 mM arginine and 0.01% PS80, pH 6.0, shows better stability than other formulations containing NaCl or combination of NaCl, trehalose and arginine. A formulation containing 5-10 mM citrate, 117 mM trehalose, 100 mM arginine, ˜0.01% PS80, pH 5.9 to 6.0 is recommended.

1. A liquid composition comprising a polypeptide having at least 90%, 91%, 92%, 93%, 94%, 95%, 96%, 97%, 98%, 99% or 100% sequence identity to the polypeptide set forth in SEQ ID NO:1 or a polypeptide having at least 90%, 91%, 92%, 93%, 94%, 95%, 96%, 97%, 98%, 99% or 100% sequence identity to the polypeptide set forth in SEQ ID NO:1 which is truncated at the C-terminus and/or the N-terminus, at least one buffering agent, at least one saccharide and/or at least one polyol, at least one stabilizing agent and optionally at least one surfactant wherein said polypeptide remains stable in said liquid composition.

2. The liquid composition of claim 1 wherein the polypeptide is truncated at the N-terminus by 1, 2, 3, 4, 5, 6, 7, 8, 9, or 10 amino acids compared to SEQ ID NO:1 or a polypeptide having at least 90%, 91%, 92%, 93%, 94%, 95%, 96%, 97%, 98%, 99% or 100% sequence identity to SEQ ID NO:1 over the entire sequence.

3. The liquid composition of claim 1 wherein said polypeptide is truncated at the C-terminus by 1, 2, 3, 4, 5, 6, 7, 8, 9, or 10 amino acids compared to SEQ ID NO:1 or and polypeptide having at least 90%, 91%, 92%, 93%, 94%, 95%, 96%, 97%, 98%, 99% or 100% sequence identity to SEQ ID NO:1 over the entire sequence.

4. The liquid composition of claim 1 wherein said buffering agent comprises sodium citrate at a concentration of about 5 mM to about 15 mM in said liquid composition.

5. The liquid composition of claim 1 wherein said buffering agent comprises succinate at a concentration of about 5 mM to about 10 mM in said liquid composition.

6. The liquid composition of claim 1 wherein said liquid composition comprises at least one saccharide at a concentration of about 72 mM to about 207 mM in said liquid composition.

7. The liquid composition of claim 1 wherein said stabilizing agent comprises arginine at a concentration of about 50 mM to about 125 mM in said liquid composition.

8. The liquid composition of claim 1 wherein said stabilizing agent or said buffering agent comprises histidine at a concentration of about 50 mM to about 125 mM in said liquid composition.

9. The liquid composition of claim 1 wherein the stabilizing agent comprises sucrose.

10. The liquid composition of claim 1 wherein said liquid composition comprises a surfactant.

11. The liquid composition of claim 10 wherein said surfactant is selected from polysorbate 80 and polysorbate 20.

12. The liquid composition of claim 1 comprising sodium citrate, trehalose, arginine, polysorbate 80 and water wherein said composition has a pH of about 5.5 to about 6.0.

13. The liquid composition of claim 1 comprising about 30 mg/mL to about 100 mg/mL of a polypeptide having the amino acid sequence set forth in SEQ ID NO:1, about 110 mM to about 140 mM trehalose, 90 mM to about 110 mM arginine, about 5 mM to about 15 mM sodium citrate, and 0.01% w/w polysorbate 80 wherein said composition has a pH of about 5.5 to about 6.0.

14. The liquid composition of claim 1 comprising about 50 mg/mL of a polypeptide having the amino acid sequence set forth in SEQ ID NO:1.

15. The liquid composition of claim 1 consisting of 50 mg/mL of a polypeptide having the amino acid sequence set forth in SEQ ID NO:1, 117 mM trehalose, 100 mM arginine, 10 mM sodium citrate, 0.01% w/w polysorbate 80 and water for injection wherein said composition has a pH of about 5.9.

16. The liquid composition of claim 1 wherein said polypeptide has at least one GLP-1 activity and maintains said GLP-1 activity in said liquid composition for at least one week.

17. The liquid composition of claim 1 wherein at least 96% of said polypeptide remains as a monomer in said liquid composition for at least a week.

18. The liquid composition of claim 1 wherein said polypeptide has at least one GLP-1 activity and maintains said at least one GLP-1 activity at at least 90% potency for at least 12 months when protected from light.

19. The liquid composition of claim 1 were said polypeptide remains stable in said liquid composition for at least 12 months when the composition is maintained at about 2° C. to about 8° C. when protected from light.

20. A method of providing glycemic control to a human in need thereof comprising administering to said human the liquid composition of claim 1.
